(19) United States
(12) Patent Application Publication
(10) Pub. No.: US 2020/0411144 A1
(43) Pub. Date: Dec. 31, 2020

(54) SPARSE CO-VARYING UNIT OF THE HUMAN GUT MICROBIOTA THAT DESCRIBES HEALTHY AND IMPAIRED COMMUNITY DEVELOPMENT

(71) Applicants: Arjun RAMAN, St. Louis, MO (US); Jeffrey Gordon, St. Louis, MO (US)

(72) Inventors: Arjun RAMAN, St. Louis, MO (US); Jeffrey Gordon, St. Louis, MO (US)

(73) Assignee: Washington University, St. Louis, MO (US)

(21) Appl. No.: 16/946,215

(22) Filed: Jun. 10, 2020

Related U.S. Application Data
(60) Provisional application No. 62/859,455, filed on Jun. 10, 2019.

(51) Int. Cl. G16H 10/40; A61B 5/00; G16B 40/00; G16B 50/00; G16H 20/00; G16H 40/63; G16H 50/30

(52) U.S. Cl. CPC G16H 10/40; A61B 5/4211; A61B 5/4839; G16H 50/30; G16B 50/00; G16H 20/00; G16H 40/63; G16B 40/00

(57) ABSTRACT

Characterizing the organization of microbial communities is a formidable challenge given the number of possible interactions between their components. Using a statistical approach initially applied to financial markets, we measured covariance among bacterial taxa in the gut microbiota of healthy members of a Bangladeshi birth cohort sampled monthly from 1-60 months and identified an ‘ecogroup’ composed of 15 co-varying bacterial taxa. A distinct ecogroup configuration is evident by the second postnatal month and develops to a mature form by 21 months. The ‘ecogroup’ provided a concise description of microbiota organization in healthy members of birth cohorts from several low-income countries, a means for monitoring community repair in undernourished children treated with therapeutic foods and serves as a framework for studying emergent characteristics of microbial communities.

### STATEMENT REGARDING FEDERALLY SPONSORED RESEARCH & DEVELOPMENT

This invention was made with government support under DK030292 awarded by the National Institutes of Health. The government has certain rights in the invention.

### BACKGROUND

Innumerable studies of the functioning of biological systems have underscored the importance of characterizing interactions between their component parts. Defining microbial communities in this way can present a seemingly intractable challenge. For example, the gut of a healthy adult human harbors multiple species, with multiple strain-level variants of a given species that can engage in higher-order interactions with other community members. Using a conservative species count of 100, the number of terms needed to mathematically represent all possible species-species interactions (pairwise and higher-order) is ˜10³⁰ . Given this potential complexity, the identification of interactions between component members that provide a simplified description of a community and reduce the number of features needed for characterization of community properties, such as its assembly after birth or responses to various perturbations is an on-going challenge. Approaches developed in the fields of econophysics and protein evolution that apply the concept of covariance to financial markets and protein families have identified cofluctuating economic sectors and cooperative amino acid networks of functional relevance, respectively. The application of covariance approaches to microbial communities may similarly provide a potential means of characterizing interactions amongst individual species of such communities.

### SUMMARY

In one aspect, a computer-implemented method for characterizing a gut microbiome of a group of subjects is described. The method includes providing a microbiome dataset that includes a plurality of entries in which each entry includes a plurality of microbial taxa and associated abundances. Each entry also includes at least one subject classification selected from an age, a health condition, a treatment condition, and a geographical location. The method further includes transforming a first portion of the microbiome dataset into a first eigenspectrum, transforming at least one additional portion of the microbiome dataset into at least one additional eigenspectrum, comparing corresponding components of the first eigenspectrum and the at least one additional eigenspectrum, and characterizing the gut microbiome based on the comparison of the first eigenspectrum and the at least one additional eigenspectrum. Each of the first eigenspectrum and the at least one additional eigenspectrum includes a plurality of eigenvectors and associated eigenvalues.

In some aspects, the method described above may further include monitoring an effect of a treatment for a gastrointestinal condition using a treatment characterized by a plurality of phases. In addition, the first portion described above may include a combination of all entries of the plurality of entries of the microbiome dataset with a health condition of healthy, and each additional portion include a combination of all entries of the plurality of entries with a health condition of gastrointestinal condition and a treatment condition classified as undergoing one phase of the plurality of phases of the treatment.

In some other aspects, monitoring the effect of the treatment as described above further includes transforming the first eigenvector and each additional eigenvector into a separation distance. In these other aspects, a reduction in separation distance between an earlier phase and a later phase of a treatment indicates an efficacy of the treatment.

In other additional aspects, characterizing the gut microbiome as described above includes identifying a microbiome configuration age to achieve a stable microbiome configuration. In these other additional aspects, the first portion described above includes a combination of all entries of the plurality of entries of the microbiome dataset that include the subject classifications of the youngest age and the oldest age, and each additional portion includes a successively larger portion of the plurality of entries. Each successively larger portion includes all entries of the plurality of entries of the microbiome dataset that include the subject classifications of the youngest age, the oldest age, and successively larger portions of the ages between the youngest age and the oldest age. Comparing corresponding components of the first eigenspectrum and the at least one additional eigenspectrum as described above includes comparing each first eigenvalue associated with each first eigenvector of each eigenspectrum. Characterizing the gut microbiome based on the comparison of the first eigenspectrum and the at least one additional eigenspectrum as described above includes identifying the stable eigenspectrum from the at least one additional eigenspectrum at which the first eigenvalue reaches an asymptotic value, and identifying the age added to generate the additional portion of the entries transformed into the stable eigenspectrum as the age to achieve a stable microbiome configuration.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following figures illustrate various aspects of the disclosure.

FIG. 1 is a heat map summarizing the fractional abundances of 8 bacterial taxa detected in fecal samples from healthy members of the Mirpur birth cohort at postnatal months 1 and 36;

FIG. 2 is a graph of a ‘Microbiota Dissimilarity Index’ obtained using iterative PCA of the fractional abundances summarized in FIG. 1;

FIG. 3 is a schematic diagram illustrating a method for converting monthly abundance distributions of bacterial taxa to a temporally weighted covariance matrix;

FIG. 4 is a heat map summarizing temporally weighted covariance values for each taxon pair) calculated using the method of illustrated in FIG. 4 on the fractional abundances of fecal samples from healthy members of the Mirpur birth cohort at postnatal months 1 through 60;

FIG. 5A is a histogram summarizing a projection of the bacterial taxa of FIG. 4 along principal components axis PC1;

FIG. 5B is an eigendecomposition of the histogram of FIG. 5A;

FIG. 6 is a heat map showing hierarchical clustering of the covariance matrix (<C_{bin}^{i,j}>) shown in FIG. 4;

FIG. 7 is an ecogroup network map for the bacterial taxa of FIG. 6 mapping the covariance for taxa pairs with <C_{bin}^{i,j}> values within the top 20% of all <C_{bin}^{i,j}> values, with all nodes sized in proportion to number of intersecting edges and all edges weighted in proportion to the [symbol] C_{bin}^{i,j} [symbol] values;

FIG. 8A is a diagram illustrating the clustering of five ecogroup configurations of bacterial taxa detected within fecal samples obtained from the healthy members of the Mirpur birth cohort at postnatal month 1, as projected onto principal components axes PC1, PC2, and PC3;

FIG. 8B is a diagram illustrating the clustering of five ecogroup configurations of the bacterial taxa of FIG. 8A, as projected onto principal components axes PC1 and PC2;

FIG. 9A is a heat map of fractional abundances of the bacterial taxa within ecogroups 1-5 of FIGS. 8A and 8B at postnatal month 1;

FIG. 9B is a heat map of fractional abundances of the bacterial taxa within ecogroups 1-4 of FIGS. 8A and 8B at postnatal month 2, and within ecogroup 5 at postnatal month 4;

FIG. 10A is a diagram summarizing the development of ecogroup configuration 1 over post-natal months 1-23 in terms of microbiota maturation index and ecogroup network maps;

FIG. 10B is a diagram summarizing the development of ecogroup configurations 2, 3, and 4 over post-natal months 1-27 in terms of microbiota maturation index and ecogroup network maps;

FIG. 10C is a diagram summarizing the development of ecogroup configuration 5 over post-natal months 1-26 in terms of microbiota maturation index and ecogroup network maps;

FIG. 11 is a diagram illustrating the clustering of the bacterial taxa detected within fecal samples obtained from 3 age groups within the healthy members of the Mirpur birth cohort, as projected onto principal components axes PC1, PC2, and PC3, as well as ecogroup network maps representative of each age group;

FIG. 12 is a diagram illustrating the clustering of the bacterial taxa detected within fecal samples obtained from 3 age groups within a sub-population of the Mirpur birth cohort with moderate acute malnutrition (MAM), as projected onto principal components axes PC1, PC2, and PC3, as well as ecogroup network maps representative of each age group;

FIG. 13 contains a series of microbiota configurations projected onto principal components axes PC1, PC2, and PC3 for a sub-population of the Mirpur birth cohort with moderate acute malnutrition (MAM) before and after the administration of microbiota-directed complementary foods (MDCFs);

FIG. 14 contains a series of bar graphs summarizing the microbiota configurations of FIG. 13 projected onto principal components axes PC1 and PC2;

FIG. 15 is a graph showing the centroid of the microbiota configurations projected onto principal components axes PC1, PC2, and PC3 before (dashed symbols) and after (solid symbols) administration of the microbiota-directed complementary foods (MDCFs) as illustrated in FIG. 13;

FIG. 16A is an ecogroup network map of the 20-25 month ecogroup network in untreated healthy subjects;

FIG. 16B contains heat maps summarizing bacterial abundance for the microbiota configurations of each treatment group of FIG. 13, as well as representative ecogroup network maps;

FIG. 17A is a diagram of the microbiota of healthy children (controls) and children with SAM before treatment;

FIG. 17B is a diagram of the microbiota of healthy children (controls) and children with SAM at various stages of treatment.

FIG. 18A is a diagram illustrating a process of comparing taxonomic assignments generated by QIIME and Amplicon Sequence Variants (ASVs) using DADA2;

FIG. 18B is a bar graph summarizing the probability of detecting a correct ASV for 15 randomly chosen primary OTU sequences over 10 iterations of the process illustrated in FIG. 18A;

FIG. 18C is a bar graph summarizing the probability of detecting a correct ASV for 15 primary OTU sequences corresponding to the ecogroup taxa of FIG. 7;

FIG. 18D is a bar graph summarizing the probability of detecting a correct ASV for the primary OTU sequences of 30 taxa of a 2-year sparse Bangladeshi RF-generated model;

FIG. 19A is a graph summarizing changes in gut microbiota estimated using unweighted UniFrac in healthy members of the Mirpur birth cohort sampled monthly from postnatal months 1 through 60;

FIG. 19B is a graph summarizing changes in gut microbiota estimated using weighted UniFrac in healthy members of the Mirpur birth cohort sampled monthly from postnatal months 1 through 60;

FIG. 19C is a graph summarizing changes in gut microbiota estimated using Shannon diversity index (SDI) in healthy members of the Mirpur birth cohort sampled monthly from postnatal months 1 through 60;

FIG. 19D is a graph summarizing changes in gut microbiota estimated using phylogenetic diversity (PD) in healthy members of the Mirpur birth cohort sampled monthly from postnatal months 1 through 60;

FIG. 20A is a graph summarizing the performance of a random forest (RF) derived model as a function of the number of individuals used to train the RF-derived model;

FIG. 20B is a graph summarizing the microbiota age estimated using the RF-derived model as a function of the chronical age of the individuals within the training set;

FIG. 20C is a graph summarizing the microbiota age estimated using the RF-derived model as a function of the chronical age of the individuals within the test set;

FIG. 21 is a histogram and graph summarizing the top-ranked age-discriminatory taxa in the 5-year RF-generated model based on feature importance scores;

FIG. 22 is a heat map summarizing the monthly distribution of relative abundances of age-discriminatory taxa;

FIG. 23A is a summary of fractional abundances of microbiota species collected at post-natal months 1 and 24;

FIG. 23B is an eigendecomposition of the fractional abundance data of FIG. 23A using PCA;

FIG. 23C is a graph showing the projection of the fractional abundance data of FIG. 23A onto principal component axes PC1 And PC2;

FIG. 23D is a heat map summarizing the distribution of relative abundances of the 8 taxa shown in FIG. 23C;

FIG. 24 is a schematic diagram of an iterative PCA (iPCA) procedure;

FIG. 25A is a heat map summarizing the relative abundance of 188 bacterial taxa in fecal microbiota of healthy members of the Mirpur birth cohort at post-natal month 60;

FIG. 25B is a heat map summarizing a taxon-taxon covariance matrix superimposed with a histogram of average fractional abundances for each of the microbiota taxa of FIG. 25A;

FIG. 25C is a heat map summarizing hierarchical clustering of the taxon-taxon covariance matrix of FIG. 25B;

FIG. 25D is a histogram summarizing the covariance values normalized to the maximum covariance value from FIG. 25B;

FIG. 25E is a heat map summarizing the most covarying taxa from FIG. 25C;

FIG. 26A is a diagram illustrating steps of a method of identifying groups of co-varying taxa using PCA and assuming constant bacterial loads across all fecal samples;

FIG. 26B is a diagram illustrating steps of a method of identifying groups of co-varying taxa using PCA and assuming different bacterial loads for individual fecal samples;

FIG. 26 is a diagram illustrating steps of a method of identifying groups of co-varying taxa using PCA and assuming different bacterial loads for individual fecal samples;

FIG. 27 is a graph illustrating the effect of varying the threshold used to define ecogroup taxa as illustrated in FIG. 5A;

FIG. 28A is a heat map summarizing the degree of projection onto the PC1 axis of bacterial taxa from fecal samples taken from healthy members of the Mirpur birth cohort at postnatal months 21-36;

FIG. 28B is a bar graph summarizing the covariance of an ecogroup member from FIG. 28A, Streptococcus gallolyticus, with ecogroup (red bars) and non-ecogroup (blue bars) taxa over time;

FIG. 28C is a bar graph summarizing the covariance of all ecogroup member from FIG. 28A averages over all fecal sample times for all , Streptococcus gallolyticus, with ecogroup (red bars) and non-ecogroup (blue bars) taxa over time;

FIG. 29 contains a series of graphs illustrating changes in the fractional abundances of ecogroup taxa over time;

FIG. 30A is a diagram of sample diet profile of a healthy Mirpur cohort member;

FIG. 30B contains a heat map summarizing a probability of seeing a transition to a new diet category within the healthy Mirpur cohort;

FIG. 30C is histogram of all pixels within the heat map of FIG. 30B;

FIG. 31 is a map of dietary transitions for individuals with microbiota harboring each ecogroup configuration;

FIG. 32 contains a series of graphs showing the eigenspectra (spectra of eigenvectors (Ev))) computed using the 15 bacterial OTUs of FIG. 5A as a function of the eigenspectra computed using all 1459 bacterial OTUs identified across all monthly fecal samples within and across the indicated geographic sites;

FIG. 33A is a heat map and corresponding eigenspectrum of all fecal samples from the Indian cohort of FIG. 32 for 1459 bacterial taxa;

FIG. 33B is a heat map and corresponding eigenspectrum of all fecal samples from the Indian cohort of FIG. 32 for 30 bacterial taxa derived from a sparse random forest model;

FIG. 33C is a heat map and corresponding eigenspectrum of all fecal samples from the Indian cohort of FIG. 32 for the 15 ecogroup bacterial taxa of FIG. 5A;

FIG. 33D is a graph and accompanying zoomed-in graph showing the fractional variance of the eigenvector of a subset of all bacterial taxa within fecal samples from a Bangladesh cohort plotted against corresponding fractional variances of eigenvectors obtained using a sparse random forest model (red dots) and using the ecogroup taxa of FIG. 5A (blue dots);

FIG. 33E is a graph and accompanying zoomed-in graph showing the fractional variance of the eigenvector of a subset of all bacterial taxa within fecal samples from an India cohort plotted against corresponding fractional variances of eigenvectors obtained using a sparse random forest model (red dots) and using the ecogroup taxa of FIG. 5A (blue dots);

FIG. 33F is a graph and accompanying zoomed-in graph showing the fractional variance of the eigenvector of a subset of all bacterial taxa within fecal samples from a Peru cohort plotted against corresponding fractional variances of eigenvectors obtained using a sparse random forest model (red dots) and using the ecogroup taxa of FIG. 5A (blue dots);

FIG. 34A is a summary of a sparse random forest (RF) model of gut microbiota development over two years in healthy members of birth cohorts from Bangladesh ranked in descending order of their importance to the accuracy of the RF model;

FIG. 34B is a graph summarizing the effect of the number of children used to train the RF-based model of FIG. 34A on modeling errors as expressed by Pearson's correlation coefficient;

FIG. 34C is a graph summarizing the effect of the number of children used to train the RF-based model of FIG. 34A on modeling errors as expressed by mean squared error;

FIG. 34D is a summary of a sparse random forest (RF) model of gut microbiota development over two years in healthy members of birth cohorts from India ranked in descending order of their importance to the accuracy of the RF model;

FIG. 34E is a summary of a sparse random forest (RF) model of gut microbiota development over two years in healthy members of birth cohorts from Peru ranked in descending order of their importance to the accuracy of the RF model;

FIG. 35A is a summary of an aggregate sparse random forest (RF) model of gut microbiota development over two years in healthy members of combined birth cohorts from Peru, India, Brazil and South Africa ranked in descending order of their importance to the accuracy of the aggregate RF model;

FIG. 35B is a heat map summarizing temporal changes in the mean relative abundances of age-discriminatory OTUs from the aggregate RF model of FIG. 35A;

FIG. 35C is a table summarizing the results of reciprocal tests of the RF-derived models of FIGS. 34A, 34D, 34E, and 35A;

FIG. 36A contains heat maps comparing the fractional abundances of bacterial taxa within fecal samples from the moderate acute malnutrition (MAM) cohort, grouped by: all 945 detected bacterial taxa, 30 taxa from a 2-year RF model, 29 taxa from a 5-year RF model, and 15 ecogroup taxa from FIG. 5A;

FIG. 36B contains a graph and corresponding enlargement of eigenspectra computed from fractional abundances of the 15 ecogroup taxa of FIG. 5A (blue circles) and the 30 bacterial taxa from the 2-year RF model of FIG. 36A (red circles) plotted against the eigenspectrum computed from the fractional abundances of all 945 bacterial taxa from FIG. 36A;

FIG. 36C contains a graph and corresponding enlargement of eigenspectra computed from fractional abundances of the 15 ecogroup taxa of FIG. 5A (blue circles) and the 29 bacterial taxa from the 5-year RF model of FIG. 36A (green circles) plotted against the eigenspectrum computed from the fractional abundances of all 945 bacterial taxa from FIG. 36A;

FIG. 37A contains heat maps summarizing the relative abundances of the 15 ecogroup taxa of FIG. 5A within the MAM and healthy cohorts;

FIG. 37B is a eigenspectrum of the relative abundances of FIG. 37A;

FIG. 37C is a projection onto the principal component axes of FIG. 37B of the relative abundances of fecal samples from 30 healthy control subjects, as well as fecal samples from child from the MAM cohort prior to, during, and after treatment; blue and green boxes denote B. longum-predominant and P. copri-predominant ecogroup configurations, respectively;

FIG. 38 is a heat map summarizing fractional abundances of taxa in samples from FIG. 37A, ordered by projection onto PC1 and PC2;

FIG. 39A is a heat map summarizing fractional abundances of the 15 ecogroup taxa of FIG. 5A in the fecal microbiota of 63 MAM cohort members at week 1 (pre-treatment);

FIG. 39B contain graphs summarizing the projection of the fractional abundances from FIG. 39A onto principal component axes PC1, PC2, and PC3 with a PC1 threshold defining MAM ecogroup configuration A, B, C, and D;

FIG. 39C contains heat graphs summarizing the fractional abundances of the 15 ecogroup taxa of FIG. 5A in the fecal microbiota of MAM ecogroup configurations A, B, C, and D defined by the thresholds of FIG. 39B;

FIG. 40A is a heat map summarizing the fractional abundances of 944 taxa in the fecal samples collected over a course of treatment of a cohort of children with severe acute malnutrition (MAM) with one of three standard therapeutic foods;

FIG. 40B is a heat map summarizing the fractional abundances of the 15 ecogroup taxa of FIG. 5A in fecal samples described in FIG. 40A;

FIG. 40C is a graph of the eigenvalues derived from the fractional abundances of FIG. 40A plotted against the eigenvalues derived from the fractional abundances of FIG. 40B;

FIG. 41A is a heat map summarizing the fractional abundances of the 15 ecogroup taxa of FIG. 5A in fecal samples collected over a course of treatment of a cohort of children with severe acute malnutrition (SAM) with one of three standard therapeutic foods, in fecal samples collected from healthy members of a mild acute malnutrition (MAM) cohort post-treatment, and in fecal samples collected from healthy control subjects;

FIG. 41B is an eigenspectrum calculated from the fractional abundances of FIG. 41A;

FIG. 41C is a graph showing a projection onto the principal component axes of FIG. 41B of the relative abundances of fecal samples collected over a course of treatment of a cohort of children with severe acute malnutrition (SAM) with one of three standard therapeutic foods, in fecal samples collected from healthy members of a mild acute malnutrition (MAM) cohort post-treatment, and in fecal samples collected from healthy control subjects;

FIG. 42A is a graph showing a projection onto the principal component axes PC1, PC2, and PC3 of the fractional abundances of healthy controls as well as of the SAM cohort of FIG. 41A prior to treatment;

FIG. 42B is a graph showing a projection onto the principal component axes PC1, PC2, and PC3 of the fractional abundances of healthy controls as well as the SAM cohort of FIG. 41A at completion of treatment;

FIG. 42C is a graph showing a projection onto the principal component axes PC1, PC2, and PC3 of the fractional abundances of healthy controls as well as the SAM cohort of FIG. 41A one month after completion of treatment;

FIG. 42D is a graph showing a projection onto the principal component axes PC1, PC2, and PC3 of the fractional abundances of healthy controls as well as the SAM cohort of FIG. 41A six months after completion of treatment;

FIG. 42E is a graph showing a projection onto the principal component axes PC1, PC2, and PC3 of the fractional abundances of the SAM cohort of FIG. 41A nine months after completion of treatment and an untreated mild acute malnutrition (MAM) cohort;

FIG. 42F is a graph showing a projection onto the principal component axes PC1, PC2, and PC3 of the fractional abundances of the SAM cohort of FIG. 41A nine months after completion of treatment, and the mild acute malnutrition (MAM) cohort of FIG. 42E after treatment.

FIG. 43 is a block diagram schematically illustrating a system in accordance with one aspect of the disclosure;

FIG. 44 is a block diagram schematically illustrating a computing device in accordance with one aspect of the disclosure;

FIG. 45 is a block diagram schematically illustrating a remote or user computing device in accordance with one aspect of the disclosure; and

FIG. 46 is a block diagram schematically illustrating a server system in accordance with one aspect of the disclosure.

Those of skill in the art will understand that the drawings, described below, are for illustrative purposes only. The drawings are not intended to limit the scope of the present teachings in any way.

### DETAILED DESCRIPTION

Gut microbial communities typically include a plurality of member species, each of which harbors their own genome and time-varying transcriptome. The species within a gut microbial community may alter nutritional availability within the gut of the host and may further influence the physiological state of the host as a function of the gut microbial community's collective metabolic output.

A critical feature of biological systems is to reliably function yet adapt when faced with environmental fluctuations. An architecture of sparse but tight coupling enables rapid evolution to new functions in proteins. Studies of macro-ecosystems such as ant colonies have argued that adaptive behaviors are dependent on proper network organization. The gut microbiota must satisfy the constraints of survival: namely, withstanding insult and maintaining functionality (robustness) while still having the capacity for plasticity. ‘Embedding’ a sparse network of co-varying taxa in a larger framework of independently varying organisms could represent an elegant architectural solution developed by nature to maintain robustness while enabling adaptation.

The results presented here provide a starting point for addressing several questions. The very process of microbial community assembly (succession) is associated with ecogroup development: to what extent is the ecogroup self-organizing both during the period of initial community assembly and in response to various perturbations? Mechanistically, how do ecogroup species couple to each other? What are the genomic and expressed functional features of different ecogroup configurations? What are the habitat features that promote establishment and maintenance of ecogroup configurations? How do postnatal developmental changes in host environment drive ecogroup evolution and host-microbial symbiosis? One approach for addressing these questions experimentally could involve colonizing gnotobiotic mice with different ensembles of identified co-varying taxa.

As described in the examples below, the conserved covariance of gut bacterial taxa over time in a healthy Bangladeshi birth cohort sampled monthly for the first 5-years-of-life was calculated. The conserved covariance was used to identify a network of 15 co-varying bacterial organisms defined herein as a microbial ‘ecogroup’. A developmental pattern by which this network emerges is described and the utility of the ecogroup as a descriptor of microbiota development in members of birth cohorts from several other low-income countries is shown. Moreover, the ecogroup was used to characterize the degree to which perturbed microbiota are reconfigured in Bangladeshi children with acute malnutrition in response to several different types of therapeutic interventions. The co-varying network of microorganisms comprising the ecogroup may provide a framework for understanding the origins of microbiota function, robustness and capacity to adapt to various environments.

As described in additional examples below, a statistical approach was developed to identify a group of 15 co-varying bacterial taxa, described herein as an ‘ecogroup’. We find that the ecogroup is a conserved structural feature of the developing gut microbiota of healthy members of several birth cohorts residing in different countries. Moreover, the ecogroup can distinguish the microbiota of children with different degrees of undernutrition (SAM, MAM), and quantify the ability of their gut communities to be reconfigured towards a healthy state with a MDCF. While we have highlighted the utility of describing the microbiota through considering covarying taxa, future work will entail development of methods for further defining microbiota organization.

In various aspects, computer-implemented method for characterizing a gut microbiome of a group of subjects is described. In one aspect, the method assesses the covariances of a plurality of measurements indicative of the relative abundance or activity of the member taxa of a gut bacterial community. Typically, the covariances are determined from measurements resulting from the analysis of a plurality of samples, in which each sample is obtained from an individual from a subject group representative of a condition or state of interest. In this aspect, a series of covariance sets may be obtained, in which each covariance set is obtained at a different time. Each covariance set may be compared to identify covariances that are conserved over time. In one aspect, the bacterial taxa corresponding to the most persistent covariances over time are included in an ecogroup. Without being limited to any particular theory, the conserved covariances identified by the disclosed method are thought to be indicative of a conserved biological relationship between the corresponding bacterial taxa. In various aspects, the sets of measurements, with or without prior categorical grouping, are analyzed using the methods described herein using an approach of measuring temporally conserved covariance followed by matrix decomposition.

In various aspects, any measurement representative of the relative abundance, gene content, gene expression, and metabolic activity of gut microbial taxa may be used in the disclosed method without limitation. Non-limiting examples of measurements suitable for use in the disclosed method include genomic measurements, gene expression measurements, proteomic measurements, and metabolite measurements. Non-limiting examples of genomic measurements include shotgun sequencing of community DNA and identification of genes using methods known in the art. Non-limiting examples of suitable gene expression measurements include sequencing of cDNAs generated from expressed RNAs using methods known in the art. Non-limiting examples of suitable proteomic measurements include mass spectrometric, aptamer-based, ELISA-based or other methods known in the art for identifying and quantifying protein abundances. Non-limiting examples of suitable metabolite measurements include mass spectrometric, NMR or other methods known in the art for identifying and quantifying metabolites.

In some aspects, the measurements may be ‘grouped’ into ‘categories’ prior to further analysis. By way of one non-limiting example, genes may be organized into known metabolic or signaling pathways. In another non-limiting example, mRNAs or proteins may be mapped onto genes associated with metabolic or signaling pathways. In an additional non-limiting example, metabolites may be organized into groups based on their relationships to metabolic or signaling pathways or common chemical features.

In various aspects, temporal changes in covariance are assessed by comparing covariances obtained from at least two sets of samples obtained from a subject group at two or more different times. In one aspect, the times at which samples are collected may be selected to capture different ages or developmental stages of the subjects. In one aspect, the times at which samples are collected may be selected to capture the effects of a therapeutic intervention and are obtained prior to, during, and after the administration of the therapeutic intervention.

In various other aspects, the disclosed method may assess changes in the covariance of measurements as described above, in which the measurements are supplemented with additional measurements characterizing the hosts of the gut microbial communities. Non-limiting examples of suitable additional measurements to be assessed include gene products, metabolites, and known or candidate biomarkers of health status of each subject. In some aspects, measurements related to the therapeutic intervention may be included with the additional measurements described above.

Although the methods described herein are disclosed in terms of the effects of nutritional status of a population on the composition of gut microbial communities, the disclosed method may be used to assess changes in gut microbial communities associated with a variety of other disorders and therapeutic interventions including, but not limited to, gastroenteritis, diarrheal diseases, Crohn's disease, irritable bowel disorder, and any other suitable disorder.

Embodiments of the disclosure may be described in the general context of computer-executable instructions, such as program modules, executed by one or more computers or other devices. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other embodiments of the disclosure may include different computer-executable instructions or components having more or less functionality than illustrated and described herein. Aspects of the disclosure may also be practiced in distributed computing environments where tasks are performed by remote processing devices that are linked through a communications network. In a distributed computing environment, program modules may be located in both local and remote computer storage media including memory storage devices.

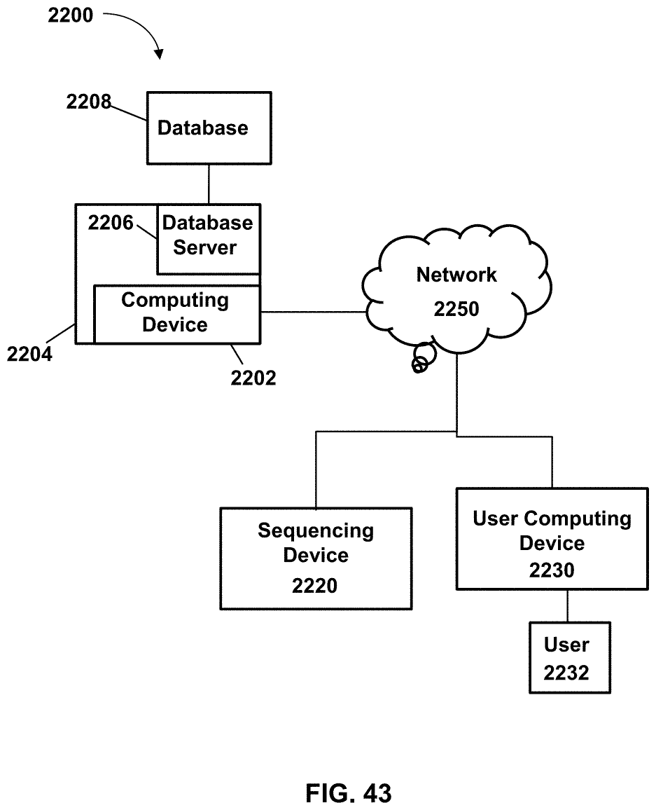

FIG. 43 depicts a simplified block diagram of a computing device 2200 for implementing the methods described herein. As illustrated in FIG. 43, the computing device 2200 may be configured to implement at least a portion of the tasks associated with obtaining and analyzing microbiota sequencing data obtained using a sequencing device 2220 including, but not limited to: operating a sequence device 2220 to obtain reads from fecal samples, analyzing the reads to obtain relative fractional abundances of OTUs, performing covariance analysis, and performing iterative principal components analysis on relative fractional abundance data and covariance data.

FIG. 43 illustrates a computer system 2200 in one aspect. The computer system 2200 may include a computing device 2202. In one aspect, the computing device 2202 is part of a server system 2204, which also includes a database server 2206. The computing device 2202 is in communication with a database 2208 through the database server 2206. The computing device 2202 is communicably coupled to the sequencing device 2220, and to a user computing device 2230 through a network 2250. The network 2250 may be any network that allows local area or wide area communication between the devices. For example, the network 2250 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 2230 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

In various aspects, the computing device 2202 is configured to operate the sequencing device 2220 to obtain and analyze a plurality of reads from fecal samples. In another aspect, the computing device 2202 is configured to analyze the reads to calculate an alpha diversity and/or a beta diversity of the taxa within the microbiome of each fecal sample including, but not limited to, the relative fractional abundance of a plurality of taxa within the microbiome. In various other aspects, computing device 2202 is configured to further transform the relative fractional abundances of one or more fecal samples to enable characterizing at least one aspect of the microbiome, including, but not limited to, covariance of taxa, microbiome configurations representative of subject populations such as healthy subjects at various developmental stages, subjects with various gastrointestinal conditions such as malnutrition, and subjects at various stages of treatment for a gastrointestinal condition.

In various additional aspects, the computing device 2204 may be configured and programmed to compare a microbiome from an individual subject to the previously-obtained microbiome configurations from one or more groups of subjects to assess for similarities or differences. In one aspect, similarities between the microbiome of the individual subject to a previously-obtained microbiome configuration may indicate membership in the subject group associated with that microbiome configuration. In another aspect, differences between the microbiome of the individual subject to a previously-obtained microbiome configuration may facilitate a diagnosis of a gastrointestinal condition in the individual. In yet another aspect, changes in the differences between the individual microbiome over the course of a treatment and a microbiome configuration microbiome may be monitored to assess an efficacy of the treatment.

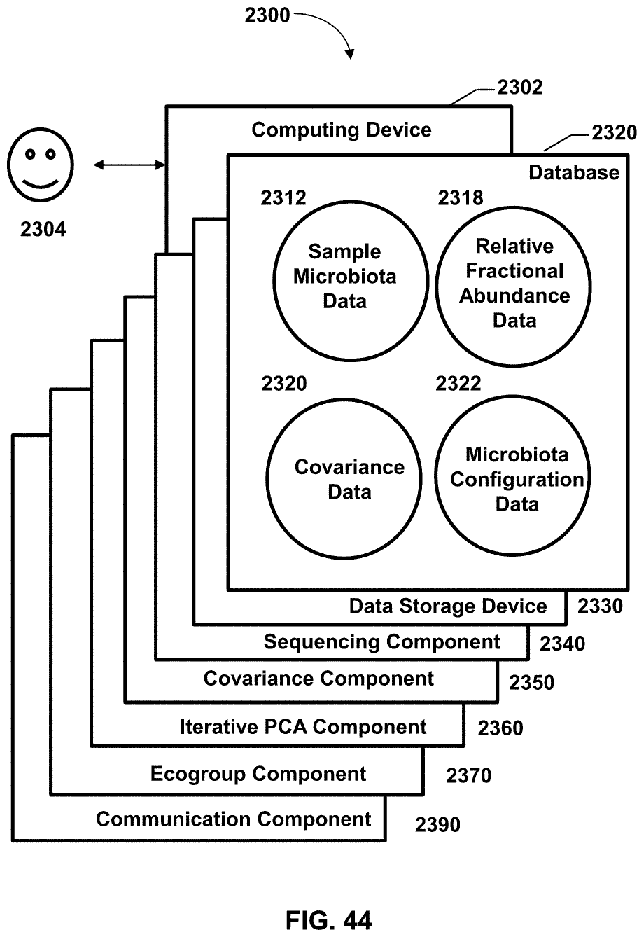

FIG. 44 depicts a component configuration 2300 of computing device 2302, which includes database 2320 along with other related computing components. In some aspects, computing device 2302 is similar to computing device 2202 (shown in FIG. 43). User 2304 may access components of computing device 802. In some aspects, database 810 is similar to database 2208 (shown in FIG. 43).

In the example aspect, database 2310 includes sample microbiota data 2312 obtained from sequencing device 2220 and/or from other sources, relative fractional abundance data 2318, covariance data 2320, and microbiota configuration data 2322.

Computing device 2302 also includes a number of components which perform specific tasks. In the example aspect, computing device 2302 includes data storage device 2330, sequencing component 2340, covariance component 2350, iterative principal components analysis (PCA) component 2360, ecogroup component 2370, and communications component 2390. Data storage device 2330 is configured to store data received or generated by computing device 2302, such as any of the data stored in database 2310 or any outputs of processes implemented by any component of computing device 2302. Sequencing component 2340 is configured to perform at least a portion of the tasks associated with sequencing and analyzing the fecal samples as described herein. In a further aspect, covariance component 2350 transforms the relative fractional abundances of one or more fecal samples into covariance matrices to compare covariance among various taxa within a sample, between various taxa between two or more samples, and the like. Iterative PCA component 2360 projects covariance data onto principal components axes, and determines eigenspectra of the principle components data. Ecogroup component 2370 is configured to select and store sub-groups of microbiota taxa characterizing a microbiota of a group of individuals as described herein. The communications component 2390 enable communications between computing device 2302 and other devices (e.g. user computing device 2230 and sequencing device 2200 shown in FIG. 43) over a network, such as network 2250 (shown in FIG. 43), or a plurality of network connections using predefined network protocols such as TCP/IP (Transmission Control Protocol/Internet Protocol).

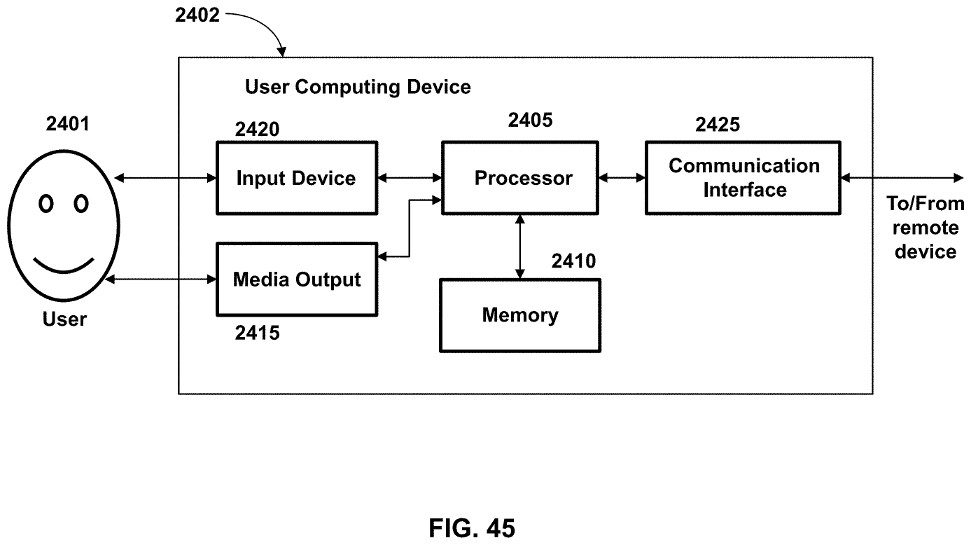

FIG. 45 depicts a configuration of a remote or user computing device 2402, such as user computing device 2230 (shown in FIG. 43). Computing device 2402 may include a processor 2405 for executing instructions. In some aspects, executable instructions may be stored in a memory area 2410. Processor 2405 may include one or more processing units (e.g., in a multi-core configuration). Memory area 2410 may be any device allowing information such as executable instructions and/or other data to be stored and retrieved. Memory area 2410 may include one or more computer-readable media.

Computing device 2402 may also include at least one media output component 2415 for presenting information to a user 2401. Media output component 2415 may be any component capable of conveying information to user 2401. In some aspects, media output component 2415 may include an output adapter, such as a video adapter and/or an audio adapter. An output adapter may be operatively coupled to processor 2405 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 2415 may be configured to present an interactive user interface (e.g., a web browser or client application) to user 2401.

In some aspects, computing device 2402 may include an input device 2420 for receiving input from user 2401. Input device 2420 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 2415 and input device 2420.

Computing device 2402 may also include a communication interface 2425, which may be communicatively coupleable to a remote device. Communication interface 2425 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 2410 are, for example, computer-readable instructions for providing a user interface to user 2401 via media output component 2415 and, optionally, receiving and processing input from input device 2420. A user interface may include, among other possibilities, a web browser and client application. Web browsers enable users 2401 to display and interact with media and other information typically embedded on a web page or a website from a web server. A client application allows users 2401 to interact with a server application associated with, for example, a vendor or business.

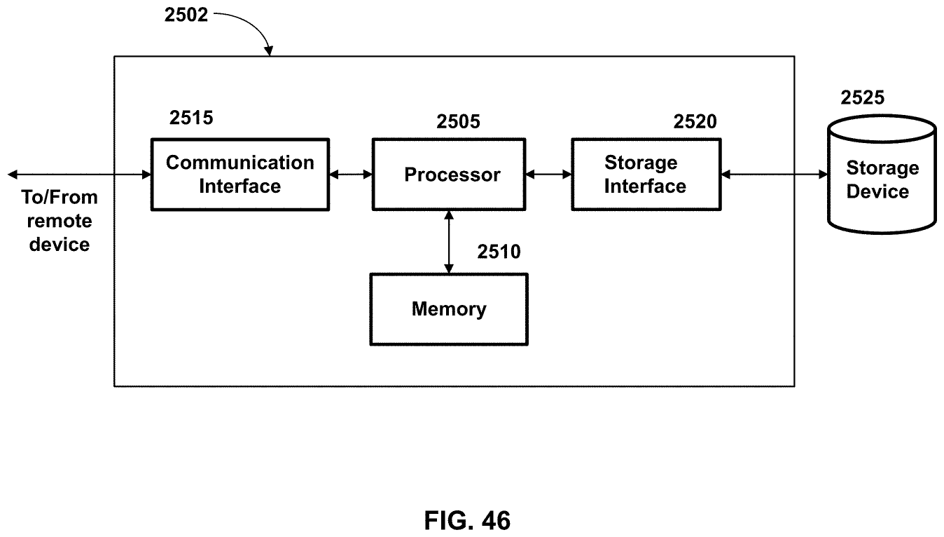

FIG. 46 illustrates an example configuration of a server system 2502. Server system 2502 may include, but is not limited to, database server 2206 and computing device 2202 (both shown in FIG. 43). In some aspects, server system 2502 is similar to server system 2204 (shown in FIG. 43). Server system 2502 may include a processor 2505 for executing instructions. Instructions may be stored in a memory area 2525, for example. Processor 2505 may include one or more processing units (e.g., in a multi-core configuration).

Processor 2505 may be operatively coupled to a communication interface 2515 such that server system 2502 may be capable of communicating with a remote device such as user computing device 2230 (shown in FIG. 43) or another server system 2502. For example, communication interface 2515 may receive requests from user computing device 2230 via a network 2250 (shown in FIG. 43).

Processor 2505 may also be operatively coupled to a storage device 2525. Storage device 2525 may be any computer-operated hardware suitable for storing and/or retrieving data. In some aspects, storage device 2525 may be integrated in server system 2502. For example, server system 2502 may include one or more hard disk drives as storage device 2525. In other aspects, storage device 2525 may be external to server system 2502 and may be accessed by a plurality of server systems 2502. For example, storage device 2525 may include multiple storage units such as hard disks or solid state disks in a redundant array of inexpensive disks (RAID) configuration. Storage device 2525 may include a storage area network (SAN) and/or a network attached storage (NAS) system.

In some aspects, processor 2505 may be operatively coupled to storage device 2525 via a storage interface 2520. Storage interface 2520 may be any component capable of providing processor 2505 with access to storage device 2525. Storage interface 2520 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 2505 with access to storage device 2525.

Memory 2410 (shown in FIG. 45) and 2510 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are example only, and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to: images or frames of a video, object characteristics, and object categorizations. Data inputs may further include: sensor data, image data, video data, telematics data, authentication data, authorization data, security data, mobile device data, geolocation information, transaction data, personal identification data, financial data, usage data, weather pattern data, “big data” sets, and/or user preference data such as sequence reads. ML outputs may include but are not limited to: a tracked shape output, categorization of an object, categorization of a type of motion, a diagnosis based on motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function which maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate a ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, a ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware or any combination or subset thereof Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one aspect, a computer program is provided, and the program is embodied on a computer readable medium. In one aspect, the system is executed on a single computer system, without requiring a connection to a sever computer. In a further aspect, the system is being run in a Windows® environment (Windows is a registered trademark of Microsoft Corporation, Redmond, Wash.). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Identifying the Ecogroup

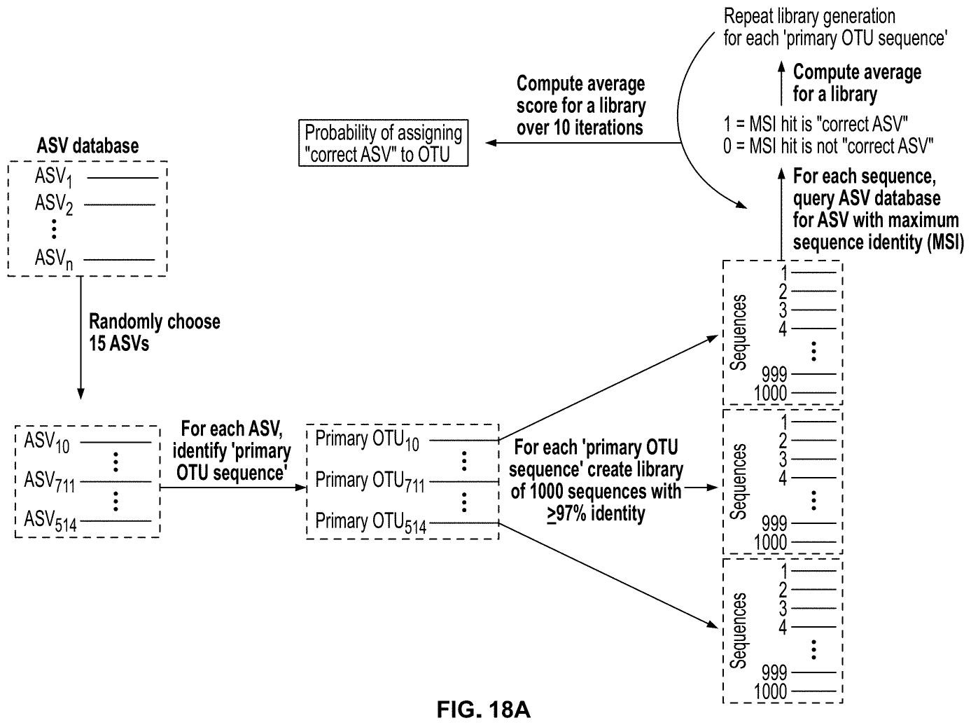

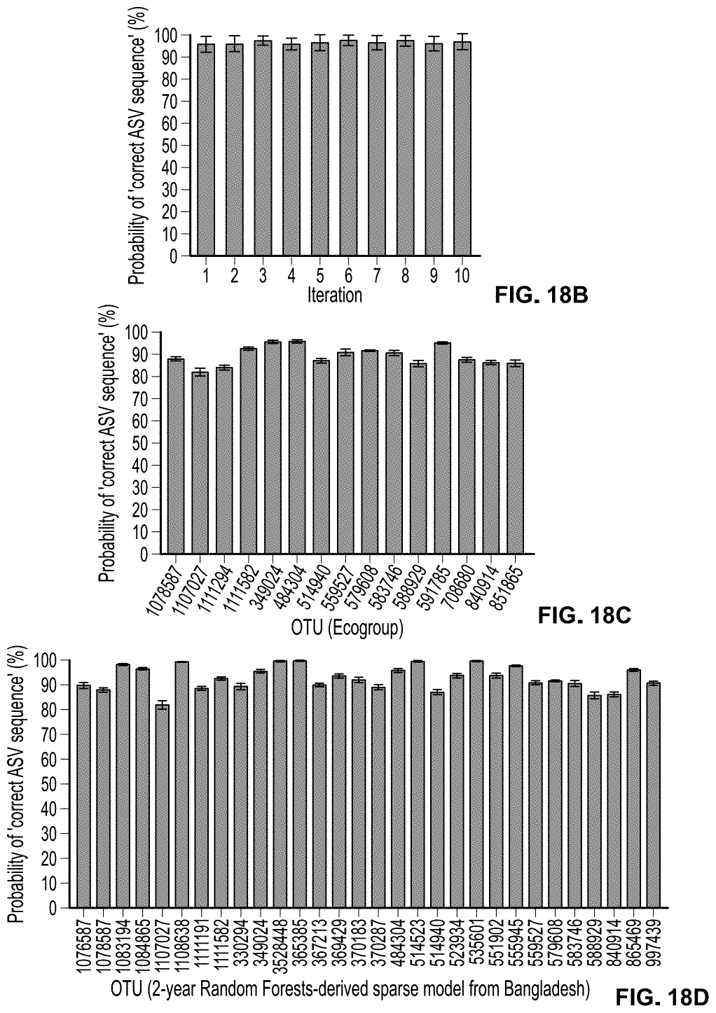

Thirty-six members of a birth cohort with consistently healthy anthropometric scores living within the Mirpur district (thana) of Dhaka, Bangladesh underwent monthly fecal sampling from 1 through 60 months [height-for-age Z-score (HAZ), −0.92±1.19 (mean±SD); weight-for-height Z-score (WHZ), −0.48±1.33 (mean±SD); n=1961 fecal samples, 55±4 samples collected/individual]. In this study population, the median duration of exclusive breastfeeding is 4 months, while the weaning process is long (median of 25 months). Samples collected less frequently, or only after 36 months, from 19 other healthy children from Mirpur were also included in our analysis (HAZ, −0.58±1.12; WHZ, −0.25±0.96; n=25.7 ±10.5 samples/child). Amplicons generated from variable region 4 (V4) of bacterial 16S rRNA genes present in these 2455 fecal samples were sequenced and the resulting reads were assigned to operational taxonomic units with >97% nucleotide sequence identity (97% ID OTUs). In total, 118 97% ID OTUs were represented at a relative fractional abundance of at least 0.001 in at least two of the samples collected over the 60-month period (also see FIG. 18).

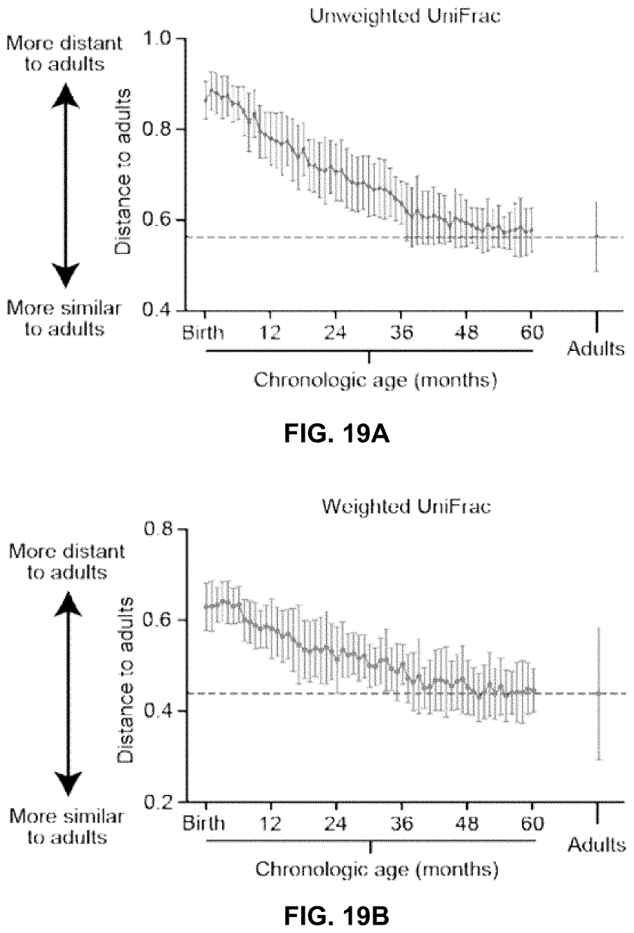

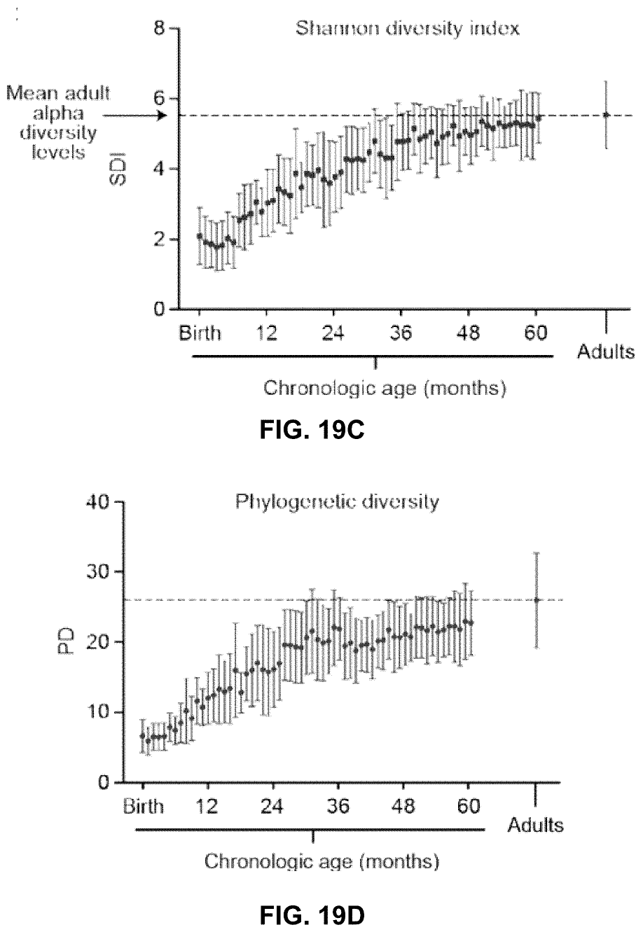

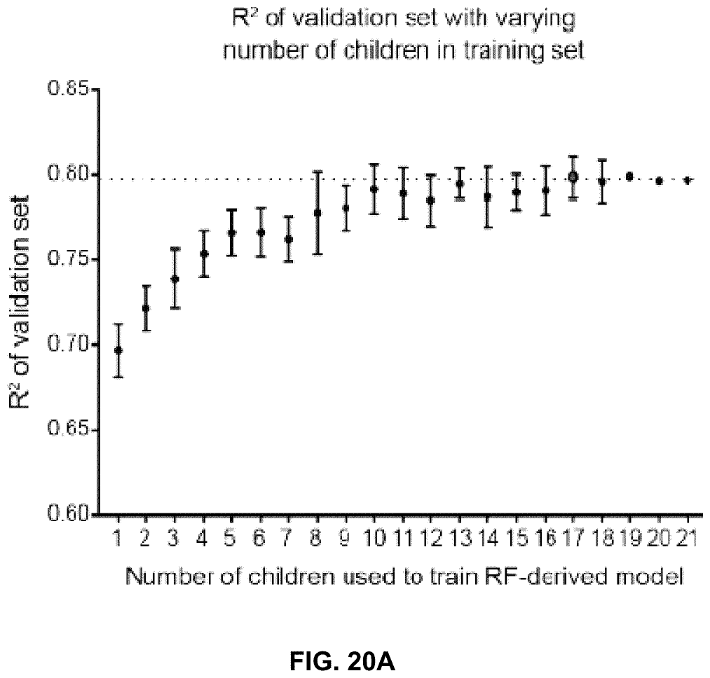

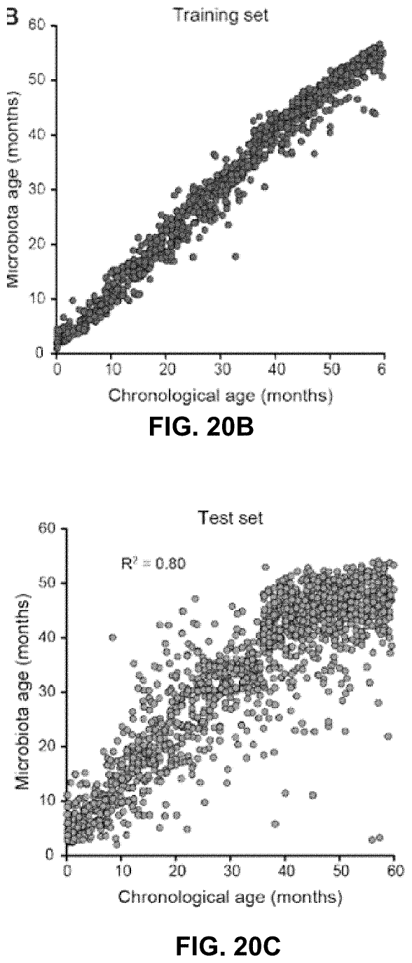

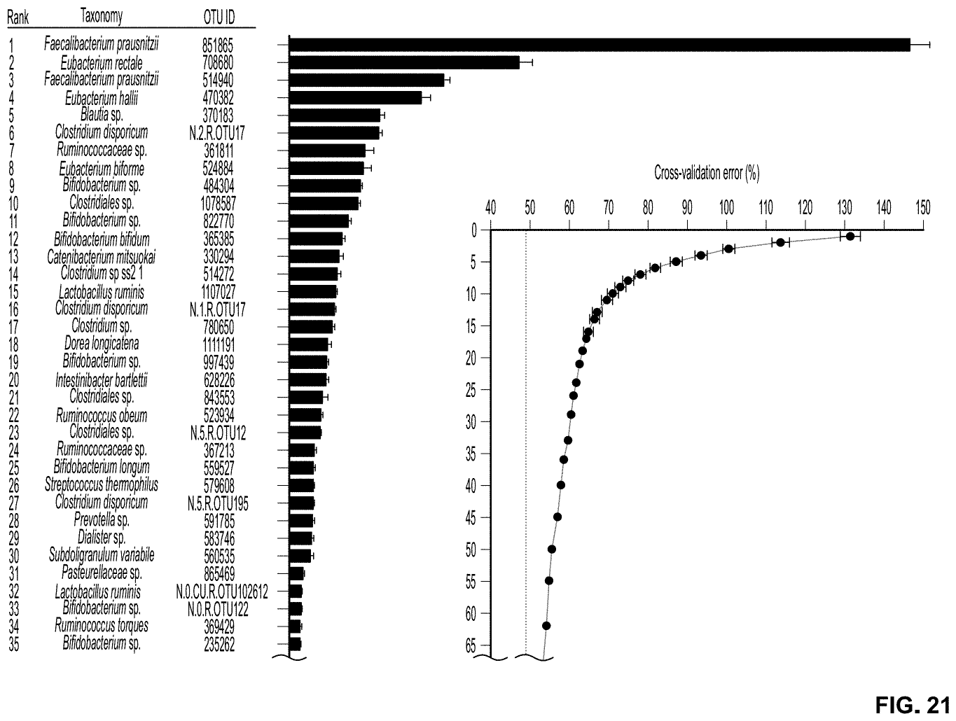

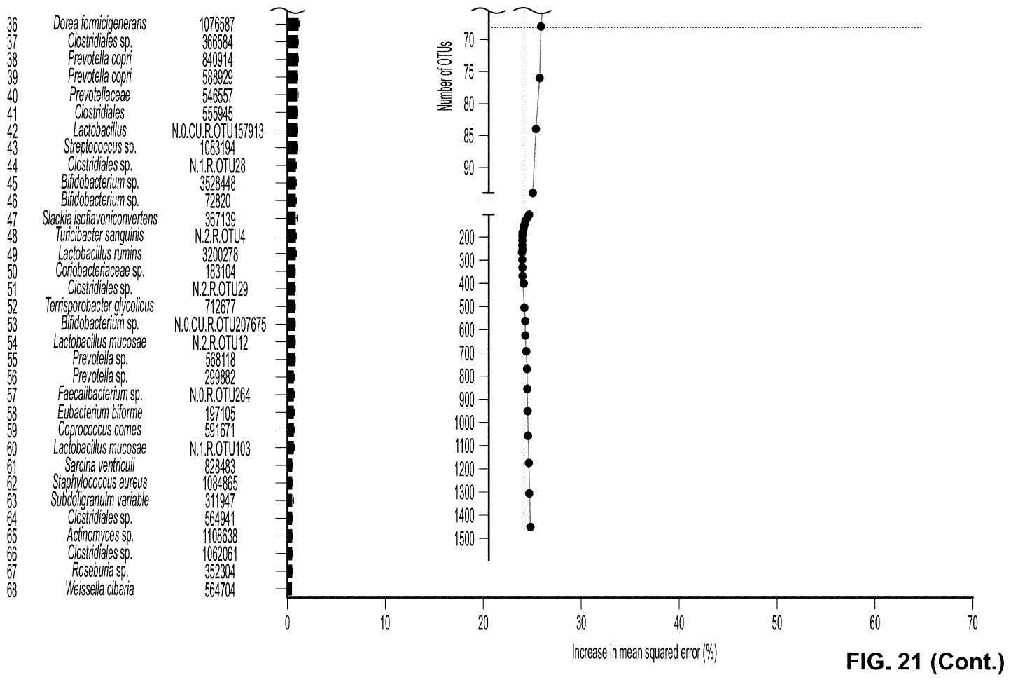

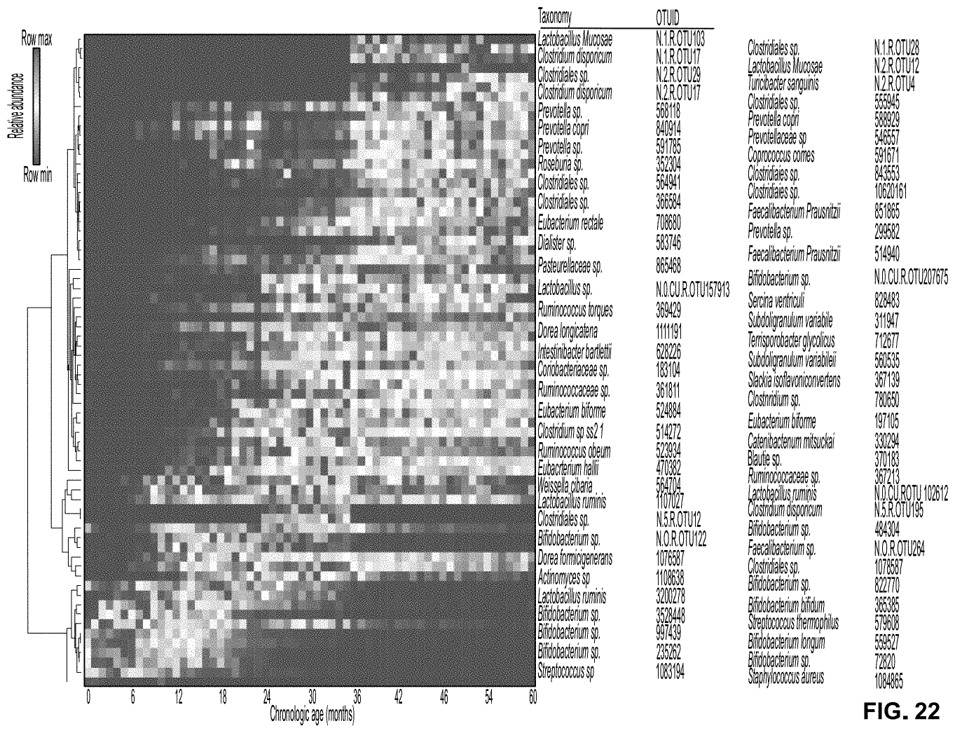

An initial broad description of microbiota development in this cohort was obtained by applying unweighted and weighted UniFrac to compute overall phylogenetic dissimilarity between gut communities from the 36 children sampled monthly from 1-60 months and 49 fecal samples collected in a previous study from 12 unrelated adults, aged 23-41 years, living in Mirpur. This metric indicated that the mean ‘infant/child-to-adult’ distance decreases to ‘adult-to-adult’ levels by 3 years of age (FIGS. 19A and 19B). Alpha diversity also increased to adult-like levels during this time period (FIGS. 19C and 19D). To provide an additional description of community development, we constructed a sparse Random Forests (RF)-derived model based on this dataset (FIGS. 20A, 20B, 20C, 21, and 22). Applying the model disclosed a high degree of correlation between microbiota age and chronologic age (R²=0.8; FIG. 20C). However, since RF provides a list of age-discriminatory taxa (FIGS. 21 and 22) without accounting for interactions within a developing community, we sought a way of characterizing community assembly that considered interactions between members.

FIGS. 19A, 19B, 19C, and 19D summarize gut microbiota development in healthy members of the Mirpur birth cohort sampled monthly from postnatal months 1 through 60. UniFrac, a beta-diversity dissimilarity metric that measures the degree to which any two communities share branch length on a bacterial phylogenetic tree, was used to calculate the degree of dissimilarity between each sampled child's fecal microbiota at each time point of fecal collection (n=36 individuals; 1961 samples) relative to samples profiled from unrelated adults who also lived in the Mirpur slum (n=12 males, 49 samples). Unweighted (FIG. 19A) and weighted UniFrac (FIG. 19B) distances are plotted as mean values±SD. As a reference control, the distances between adult samples relative to one another are shown. Alpha-diversity metrics [Shannon diversity index (SDI) and Phylogenetic diversity (PD)] plotted as mean values±SD for each monthly age bin and for adult samples in FIGS. 19C and 19D, respectively.

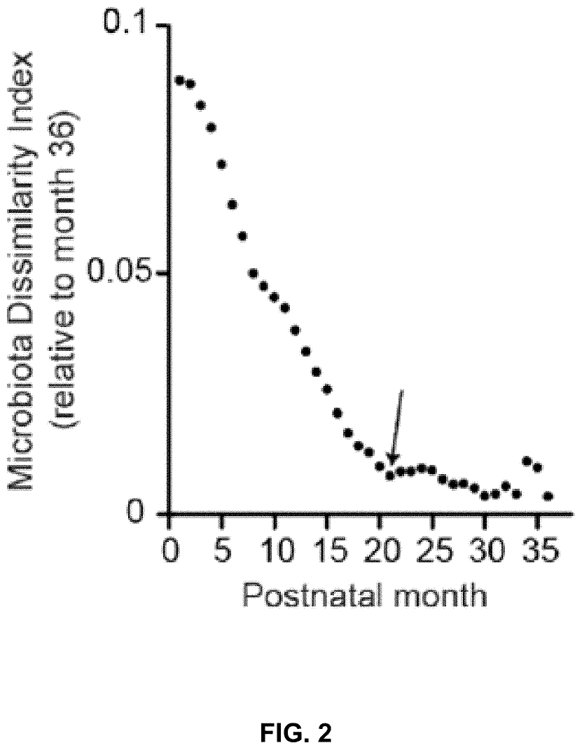

FIGS. 20A, 20B, 20C, 21, and 22 illustrate aspects of a RF-derived model of gut microbiota development based on a 60-month period of monthly sampling of healthy members of a Mirpur birth cohort. FIG. 20A illustrates the performance of RF-derived models (based on R² of validation set) with varying number of individuals in the training set. The R² reaches its optimal maximum with 17 subjects; therefore, 17 individuals were included in the training set for the final RF-derived model. FIGS. 2TB and 20C illustrate the training and validation of the 5-year RF-derived model. Each point represents a fecal sample collected from a child randomized to the training set (FIG. 20B) and validation set (FIG. 20C). FIG. 21 summarizes the top-ranked age-discriminatory taxa in the 5-year RF-generated model based on feature importance scores. FIG. 22 is a heat map of the monthly distribution of relative abundances of age-discriminatory taxa.

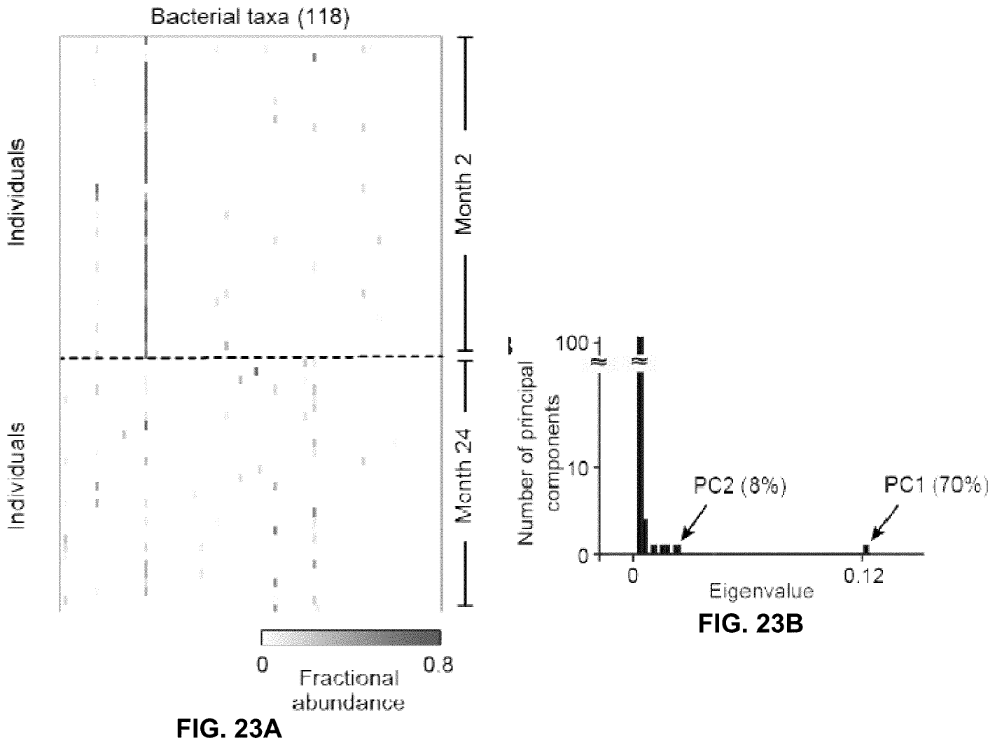

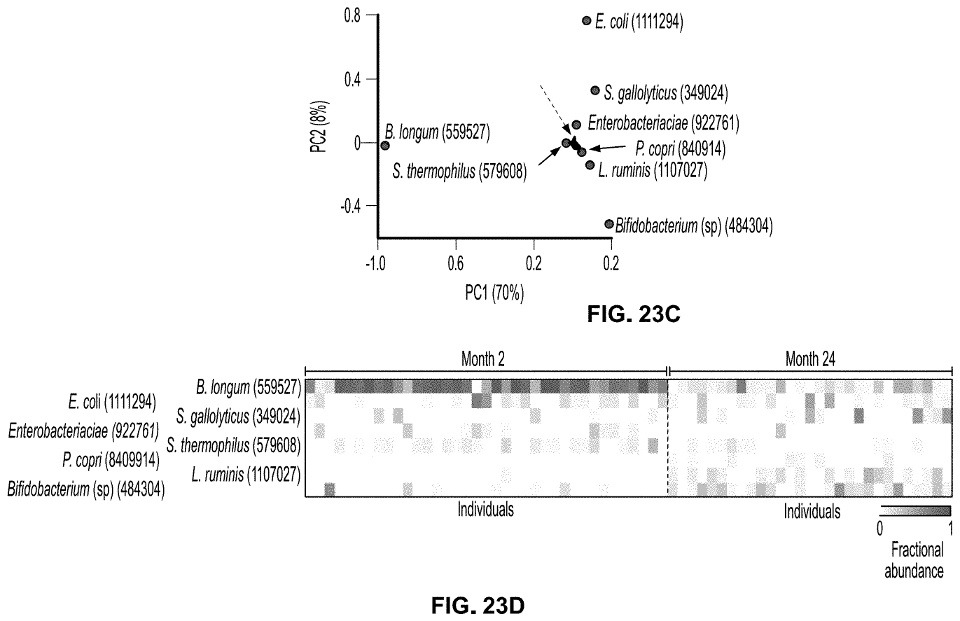

We postulated that a gut microbial community's dynamics, sampled over the first 5 years of postnatal life, reflects two temporal phases; (i) development of the community into a ‘stable’ form where interactions between taxa are being established, and (ii) subsequent temporal variations in interactions between established taxa. Therefore, we first compared the abundances of all 118 taxa in the fecal microbiota of healthy Mirpur children sampled at a time when breastfeeding is predominant (postnatal month 2) and at a time when weaning is nearing completion (month 24) (FIG. 23A). The variance in abundances of these 118 taxa was captured by principal component analysis (PCA), with the first principal component (PC1) accounting for 70% of the variation (FIG. 23B). We subsequently identified the taxa that separate the two time points by displaying their coordinates on PC1 and PC2 (FIG. 23C). The results showed that B. longum (OTU 559527) could be used to initially delineate a postnatal month 2 microbiota configuration in this cohort while a mix of seven other bacterial taxa (Escherichia coli, Streptococcus thermophilus, Streptococcus gallolyticus, Prevotella copri, Lactobacillus ruminis, a member of Enterobacteriaceae, and another Bifidobacterium sp.), could delineate a 24-month microbiota configuration (FIGS. 23C and 23D).

FIGS. 23A, 23B, 23C, and 23B illustrate the PCA-based identification of bacterial taxa that distinguish the gut microbial communities of members of the healthy Mirpur birth cohort at postnatal months 2 and 24. FIG. 23A shows the fractional abundances of 118 97%ID OTUs in microbiota collected at the two time points from the group of 36 individuals sampled monthly from postnatal months 1 to 60. Each row represents an individual's microbiota while each column represents one of the 118 taxa. FIG. 23B shows the dataset of FIG. 23A decomposed by PCA. PC1 and PC2 account for 70% and 8% of the variance, respectively. FIG. 23C is a projection of the taxa onto PC1 and PC2 reveals 8 OTUs that separate microbiota at postnatal month 2 from month 24, with B. longum responsible for separation along PC1. The dashed arrow points to the cluster of the remaining 110 taxa that do not separate along PC1 or PC2. OTU assignments are delineated in parenthesis. (D) Heat map of the distribution of relative abundances of these 8 taxa, illustrating the predominance of B. longum (OTU 559527) at month 2.

While alpha diversity continues to change through 60 months of life (FIGS. 19C and 19D), this metric provides a coarse view of microbiota composition and offers limited information regarding microbiota organization. In contrast, given many replicates of the microbiota over many time points, the spectrum of microbiota principal components offers a description of changing microbiota organization over time. Therefore, we employed an iterative implementation of Principal Components Analysis (iPCA). We considered a microbiota to be ‘mature’ if it retained a similar structure over neighboring time points. Mathematically, we measured the structure of the microbiota via decomposition of a covariance matrix, generated for a monthly set of fractional abundances of bacterial taxa, into a spectrum of principal components (eigenvectors), yielding an eigenspectrum. If microbial communities from two time points are similar, the eigenspectrum of the concatenated fractional abundances will show that top principal components contain a small amount of data variance. If two time points contain identical fractional abundances, the resulting eigenspectrum will reflect variance generated solely by the internal structure of the microbiota. In contrast, in the case where microbiota structure changes over two time points, the eigenspectrum will reflect this variance, showing at least one principal component that separates from the rest. Thus, the variance measured by the eigenspectrum quantitatively defines the similarity between microbiota collected at two time points. Given sampling over many time points, measuring the eigenspectrum over time provides a definition of system stability—the point at which the eigenspectrum reaches an asymptotic value.

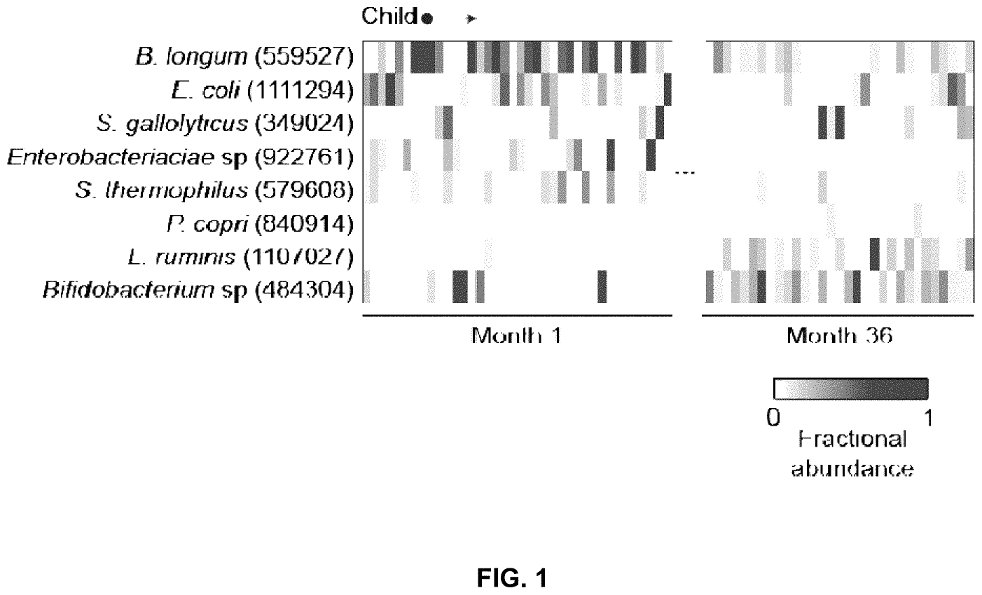

We performed iPCA on sequentially joined monthly data with month 36 taken as a reference (FIGS. 1, 2, 3, 4, 5A, and 5B). FIGS. 1-5 illustrate aspects of the workflow for defining ‘ecogroup’. FIG. 1 shows the fractional abundances of 8 bacterial taxa, identified in FIGS. 23C and 23D as distinguishing microbiota from healthy members of the Mirpur birth cohort at postnatal months 2 and 24. Fractional abundances of these taxa from postnatal month 1 to 36 are the input for iterative PCA (iPCA). Each column represents a different healthy member of the birth cohort. The set of dots (ellipsis) represents samples not depicted. As illustrated in FIG. 2 postnatal months 1 to 21 demarcate gut microbiota development, while months 22 to 60 reflect mature microbiota based on a ‘Microbiota Dissimilarity Index’ (defined by the eigenvalue of PCI obtained via iPCA).

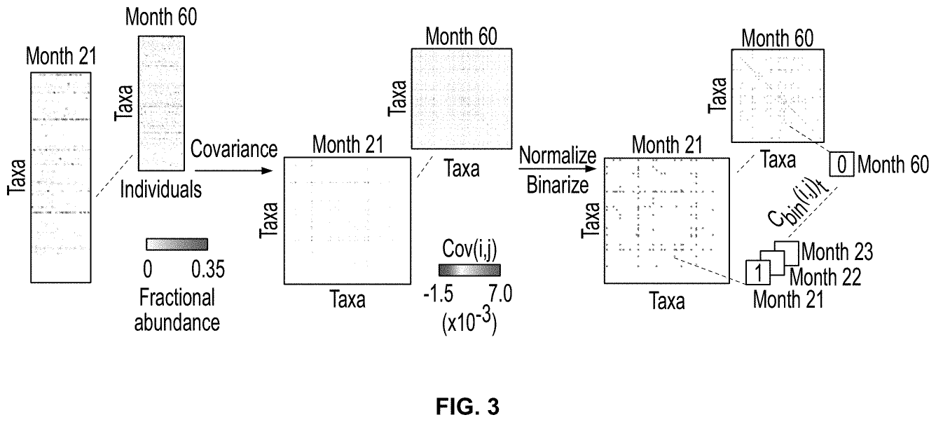

FIG. 3 illustrates a method of converting monthly abundance distributions of bacterial taxa to a temporally weighted covariance matrix ((C_{bin}^{i,j})). In the left portion of FIG. 3, 16S rDNA sequencing of fecal microbiota samples collected monthly from healthy members of the birth cohort from postnatal months 21 to 60. In the center portion of FIG. 3, covariance matrices for each month are calculated. In the right portion of FIG. 3, the monthly covariance values are normalized relative to the maximum monthly covariance value. If a normalized monthly covariance value for a given taxon-taxon pair is within the top or bottom 10% of all monthly covariance values, it is converted to a ‘1’, otherwise it is assigned a ‘0’. This binarized covariance matrix is defined asC_{bin}^{i,j}. Concatenating Cᵢₙ^{i,j} for all months creates a three-dimensional matrix (C_{bin}^{i,j}).

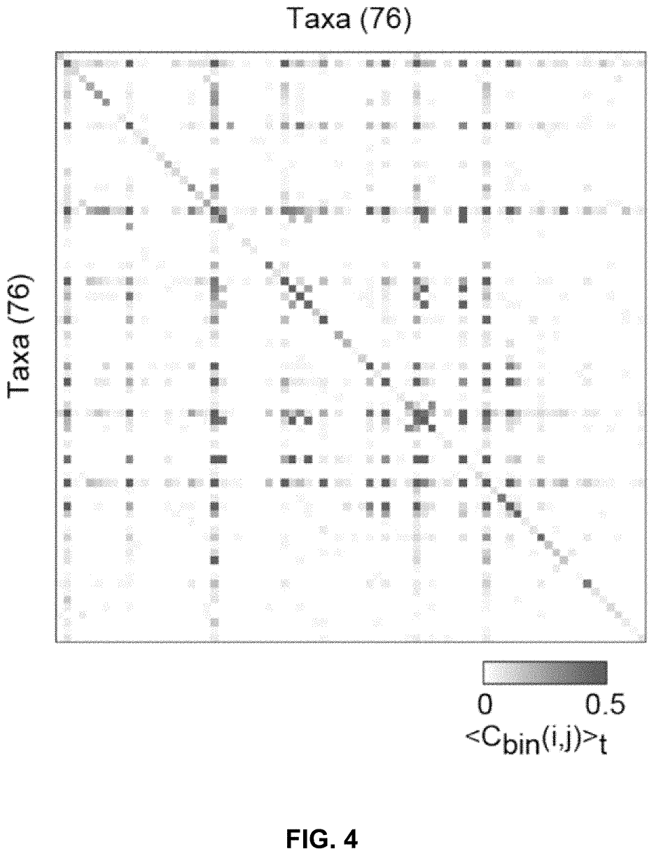

FIG. 4 shows the binarized covariance values for each (i,j) pair of taxa in (C_{bin}^{i,j})ₜ is averaged over all months to give a temporally weighted covariance value for each taxon-taxon pair ((C_{bin}^{i,j})ₜ). In the limit that two taxa always co-vary with each other, (C_{bin}^{i,j})=1. If two taxa never co-vary with each other, (C_{bin}^{i,j})=0. The matrix shown illustrates sparse temporally conserved coupling, with many taxa showing no consistent covariance ((C_{bin}^{i,j})≈0; white pixels), but a few exhibiting a high degree of conserved covariance ((C_{bin}^{i,j})≥0.5 deep red-colored pixels).

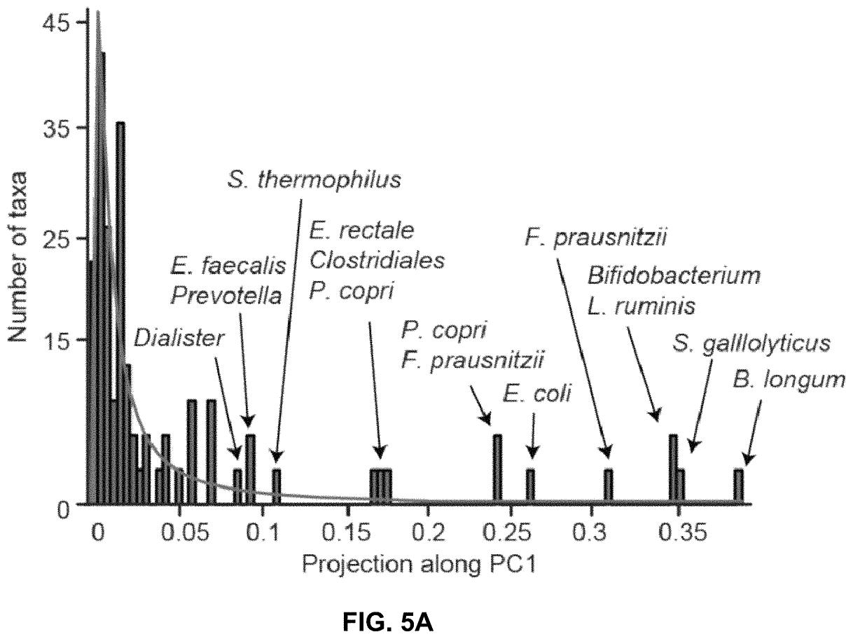

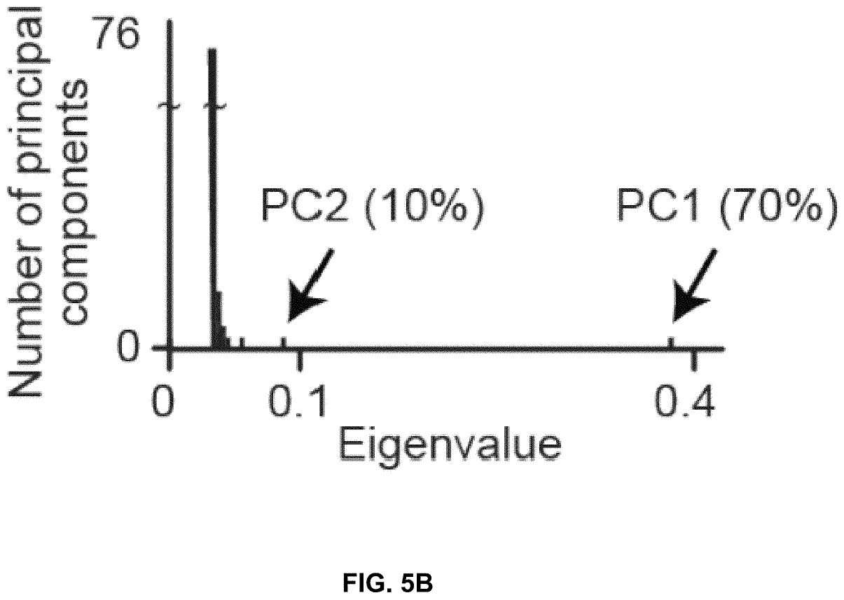

FIGS. 5A and 5B illustrates the eigendecomposition of the (C_{bin}^{i,j}) of FIG. 4, showing that 70% of the data variance in (C_{bin}^{i,j}) can be represented by a single principal component. The histogram shows projections of taxa along PC1; data are fit to a generalized extreme value distribution (red line). Applying a 20% threshold to this distribution identifies taxa that significantly contribute to the dominant mode of variance. These taxa comprise an ‘ecogroup’; i.e., organisms that co-vary with each other across time.

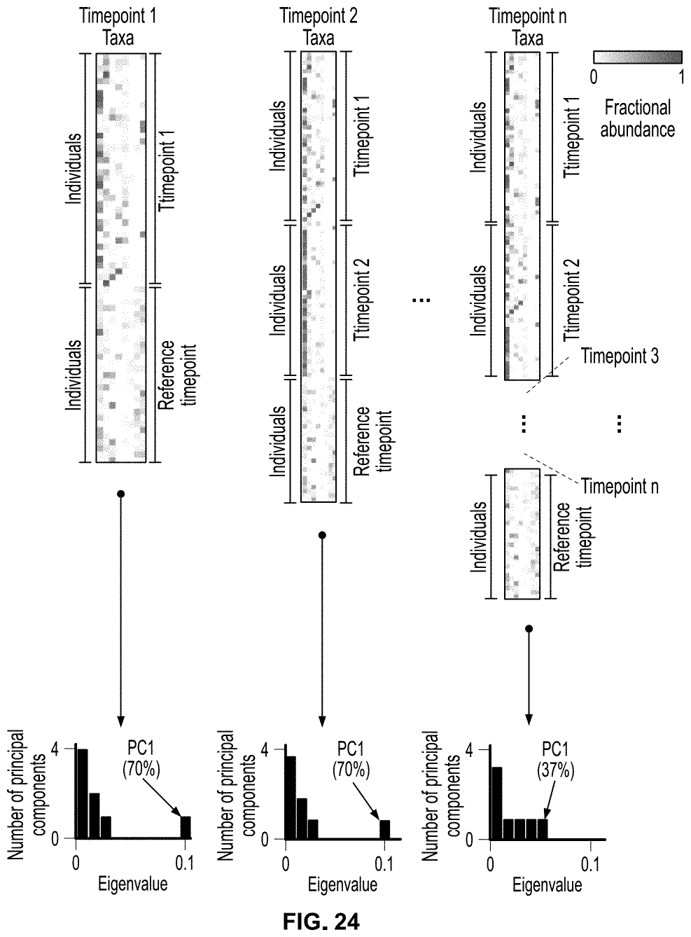

Because we sought to obtain the most general view of the dynamics of microbiota organization, we focused on the change in eigenvector 1 through time (FIG. 24). FIG. 24 is a schematic illustration of an iterative PCA (iPCA) procedure. Fractional abundances of taxa are determined in microbiota sampled from healthy members of the birth cohort at different time points. In this example, time point 1 considers two datasets (time point 1 and a reference time point). The dissimilarity between the two time points is reflected in the primary principal component (PC1). The system is considered to be stable at the time point where adding further time-series data negligibly contributes to data variance (mathematically, when the eigenvalue of PC1 reaches an asymptote).

Month 36 was chosen based on the phylogenetic dissimilarity and diversity measurements shown in FIG. 19 indicating that an adult-like configuration was achieved by this time. Month 21 and beyond demonstrate an unchanging PC1 in FIG. 2, suggesting that the gut microbiota assembles from birth to month 21 in this healthy cohort, and that the months following are reflective of dynamics within a ‘mature’ community.

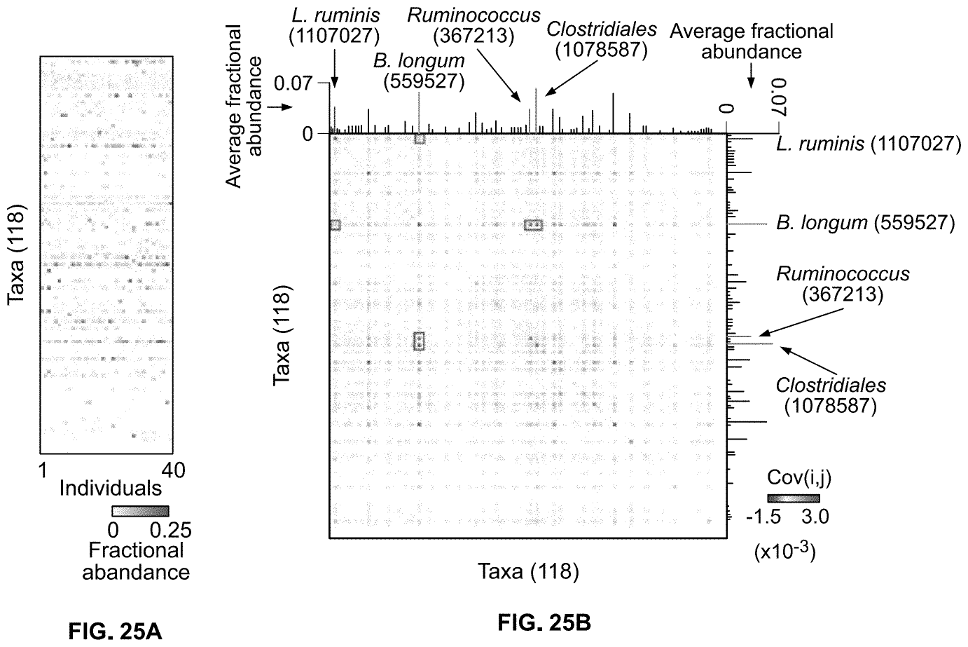

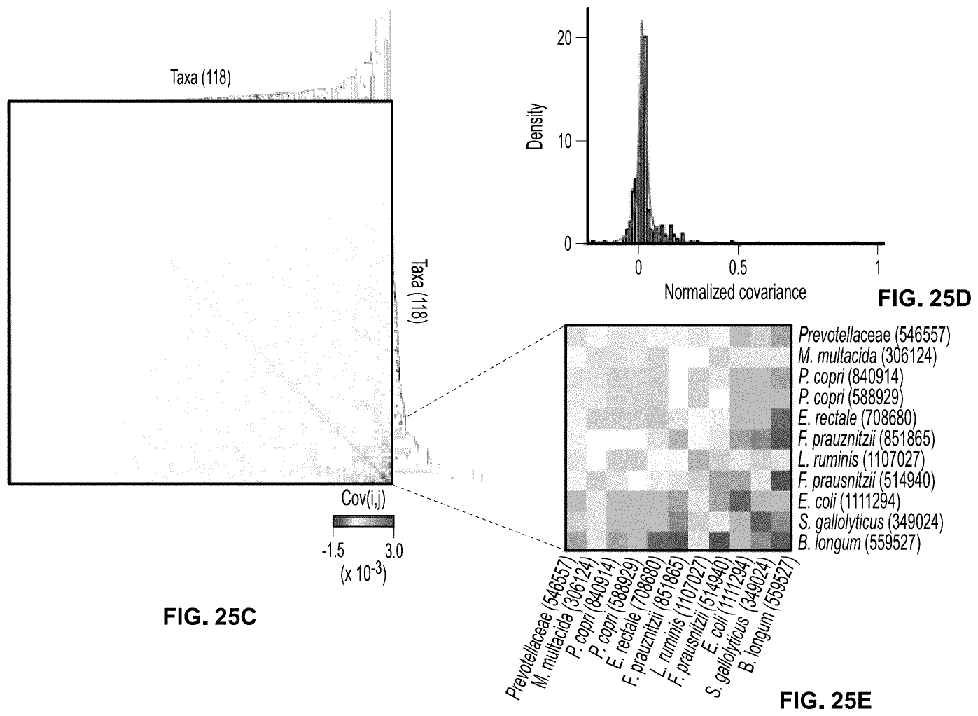

To discern a structural organization (form) of a developed microbiota, a computational workflow was designed without any a priori assumptions made about the importance of any taxa. We applied a statistical approach using covariance between taxa as a measure of mutual dependence. We focused the analysis on months 21-60, reasoning that it would allow us to measure reproducible covariance; i.e., covariance that is conserved across time in a mature community assemblage as opposed to transient covariance that may occur during community assembly (development). In addition, because covariance for any particular month could be a result of a litany of factors, a prime motivation of our approach was to weight covariance that is conserved over time. For each month, we calculated the covariance between taxa over all individuals to generate a taxon-taxon covariance matrix—a proxy for interactions between taxa (FIG. 25).

FIGS. 25A, 25B, 25C, 25D, and 25E illustrate aspects of a process for identifying co-varying bacterial taxa at postnatal month 60 in healthy members of the Mirpur birth cohort. FIG. 25A shows the fractional abundances of 118 taxa (97% ID OTUs; rows) in the fecal microbiota of healthy children (columns) sampled at postnatal month 60. FIG. 25B shows the taxon-taxon covariance matrix with superimposed distribution of average fractional abundances for each of the 118 taxa. Red, white, and blue pixels indicate positive, no, and negative covariance, respectively, between two taxa (Cov(i,j) value). As an example, B. longum (green bar) positively covaries with L. ruminis (red bar and red box) and negatively covaries with Ruminococcus and Clostridiales (blue bars and boxes). Overall, most taxa display independent variance (white pixels) with only a small subset exhibiting covariance. FIG. 25C shows the hierarchical clustering of data in FIG. 25B illustrates the sparsity of covariation within the dataset. FIG. 25D shows the covariance values are normalized against the maximum covariance value in FIG. 25B and plotted as a histogram. The red line represents a generalized extreme value distribution. The results further confirm the small number of significantly co-varying taxa. FIG. 25E shows the most co-varying taxa (highest Cov(i,j) values).

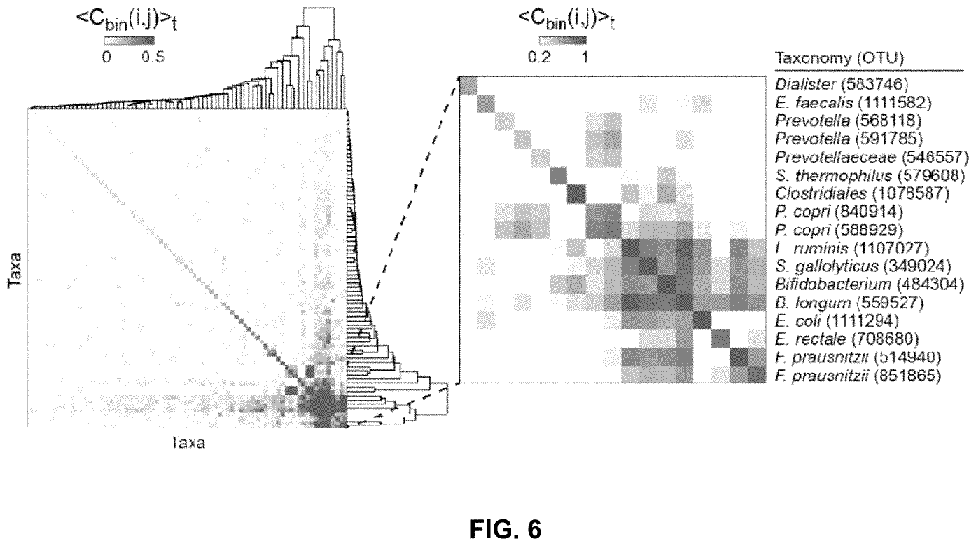

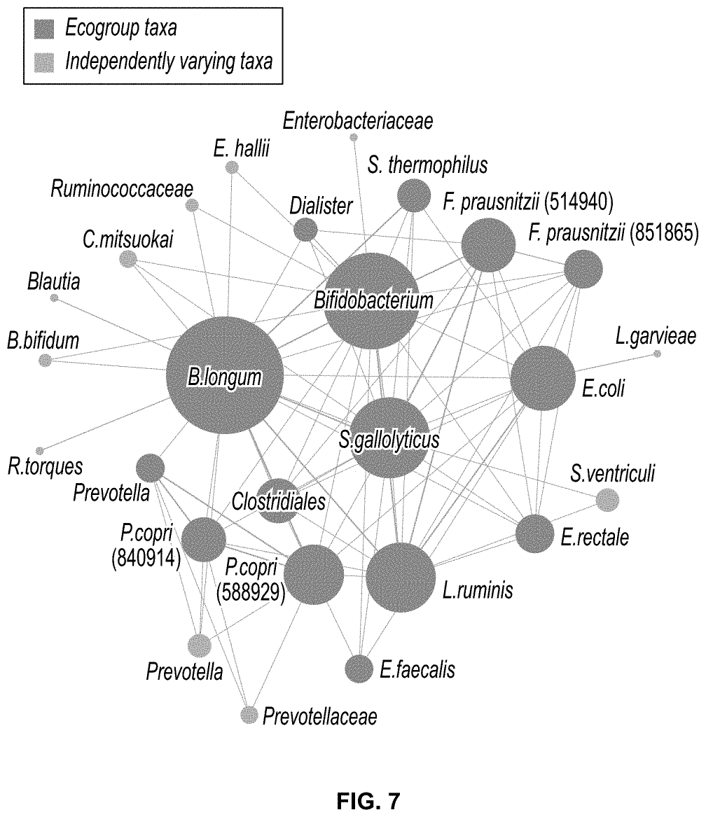

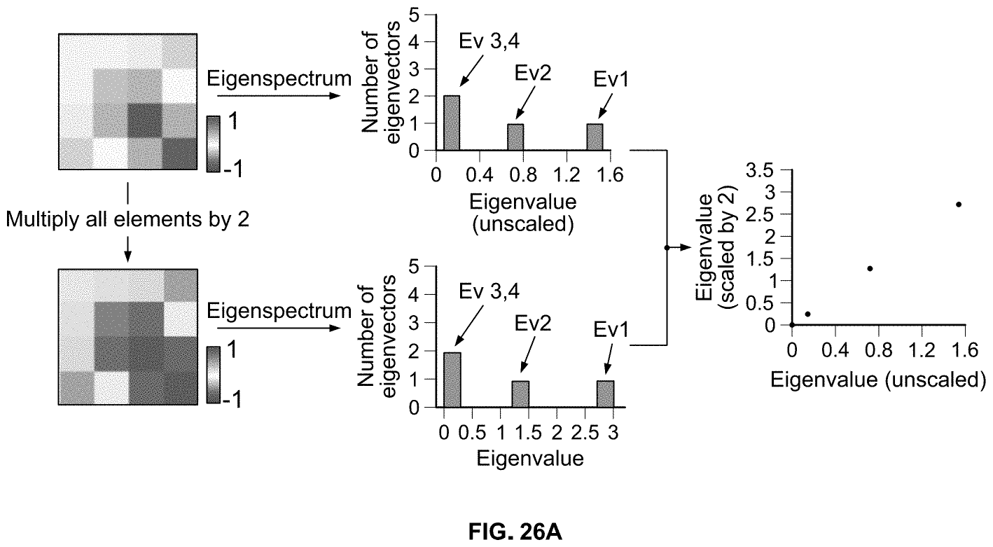

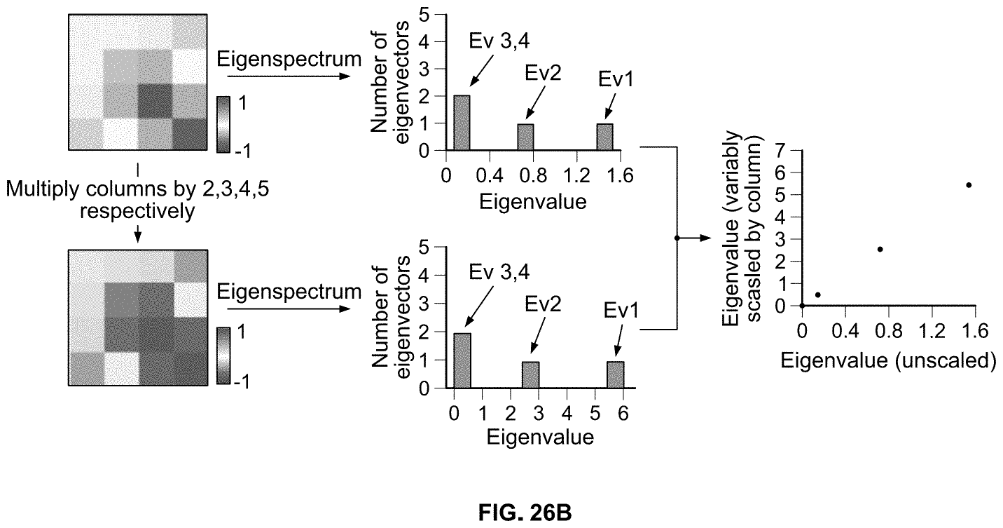

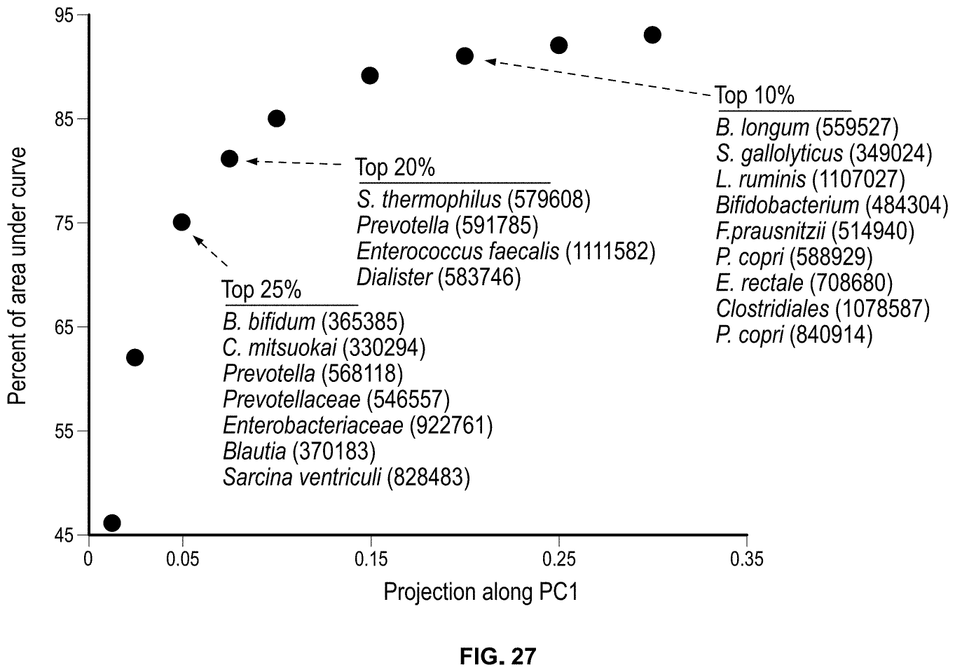

Across the healthy birth cohort from postnatal months 21 to 60, covariance matrices comprising 118 bacterial taxa for each month were first normalized against the highest covariance value within that month. As illustrated in FIG. 3 and FIG. 25D, values in the normalized covariance matrix for each postnatal month's samples were then fit to a generalized extreme value distribution and subsequently binarized according to the top and bottom 10% to isolate the most covarying taxon-taxon pairs) t, where i and j are bacterial taxa and t designates the month). For each binarized normalized monthly covariance matrix, the top 99% of taxon-taxon covariance values were retained, yielding a total of 76 taxa across months 21 to 60. These matrices were then averaged to a single 76×76 taxon-taxon matrix ((C_{bin}^{i,j})) that represents consistent covariance in the gut (fecal) microbiota of healthy Mirpur children during postnatal months 21 to 60 (FIG. 4). This 76×76 taxon matrix was then decomposed by PCA, revealing that a single dimension, PC1, contained 70% of the data variance (FIG. 5B). The computed eigenspectrum either remains unchanged or scales proportionally if bacterial load across samples is the same or varies respectively (see FIG. 26). Each taxon projects along this dimension (FIGS. 5A and 5B, blue bars) with a certain value (x-axis in FIGS. 5A and 5B). The histogram of taxa projections along PCI followed a generalized extreme value distribution (red line, FIGS. 5A and 5B). Choosing a threshold of the highest 20% PCI projections (FIG. 27) identified a group of 15 significantly co-varying bacterial taxa (FIGS. 5A and 5B). This ‘ecogroup’ includes OTUs assigned to Bifidobacterium longum, another member of Bifidobacterium, Faecalibacterium prausnitzii, Clostridiales, Prevotella copri, Streptococcus thermophilus, and Lactobacillus ruminis, all of which are age-discriminatory bacterial strains identified from RF-based analysis of bacterial 16S rRNA datasets generated from healthy members of this Bangladeshi cohort (FIG. 21). The ecogroup is an ‘insulated’ eco-structure; its members exhibit significant intragroup covariation. No members of the ecogroup are components of any other identifiable network (FIGS. 6, 7, and 28). FIGS. 6 and 7 illustrate the organization of the ecogroup. FIG. 6 shows a hierarchical clustering of the covariance matrix (<C_{bin}^{i,j}>) shown in FIG. 4, in which most taxa vary independently (white pixels). The zoom-in of FIG. 6 illustrates a high degree of covariation among the top clustered taxa. FIG. 7 is a graphical representation of co-varying bacterial taxa where two members are connected if their <C_{bin}^{i,j}> value fall within the top 20% of all <C_{bin}^{i,j}> values, in which green nodes denote the ecogroup taxa of FIG. 5. Gray nodes denote taxa outside the ecogroup. Node size is proportional to the number of connections (edges) present. The weight of each edge is proportional to the <C_{bin}^{i,j}> value between both taxa.

FIG. 27 illustrates the effect of varying the threshold used to define ecogroup taxa (see FIGS. 5A and 5B). Three thresholds (top 10%, 20%, and 25% of area under the curve (AUC), defined by the generalized extreme value distribution shown in red in FIGS. 5A and 5B, are highlighted. The taxa listed project along PC1 between the particular threshold and the next most stringent threshold. The 20% threshold defines an inflection point in the curve. Because thresholds set below 20% show a rapid decrease in AUC, the 20% threshold was chosen to define ecogroup taxa.

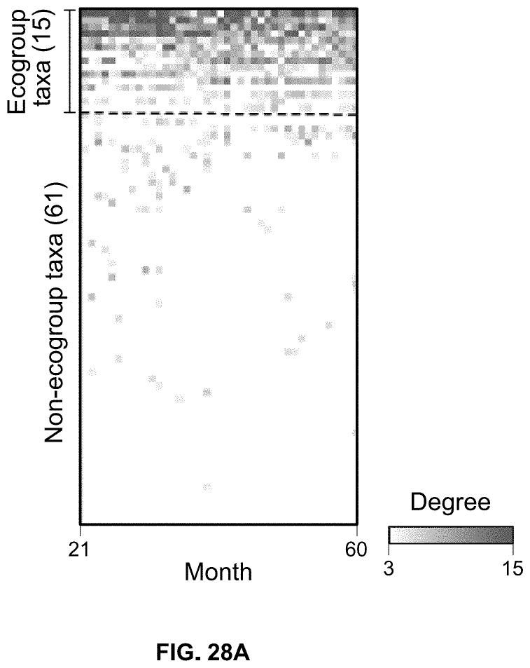

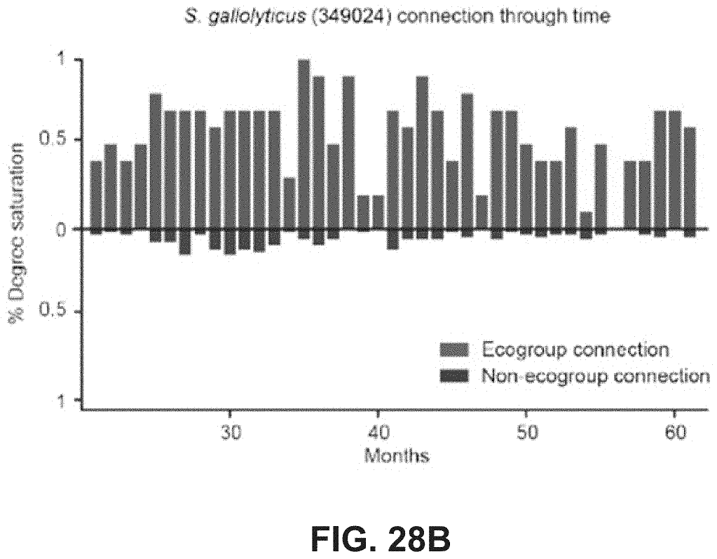

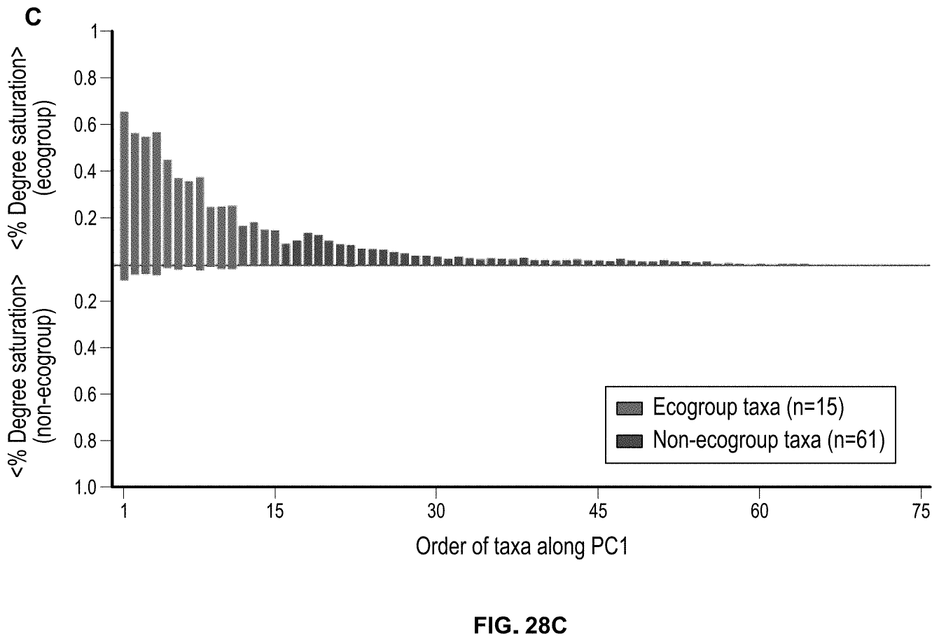

FIGS. 28A, 28B, and 28C illustrate that ecogroup taxa show high degree of coupling to other ecogroup taxa. FIG. 28A shows a matrix where columns represent the postnatal month when fecal samples were obtained from healthy members of the birth cohort, and rows represent 76 bacterial taxa ordered by the value of their projection onto PC1 as indicated in FIGS. 5A and 5B. Pixel strength represents the number of connections for a given taxon where ‘connections’ are defined as the number of significant covariance values. For network graphs such as those shown in FIGS. 5A and 5B, the number of connections for a node is termed the ‘degree’. FIG. 28B shows an example of covariance of an ecogroup member, Streptococcus gallolyticus, with ecogroup and non-ecogroup taxa over time. The y-axis plots ‘percent degree saturation’ a term that indicates number of connections observed divided by the total number of possible ecogroup connections (14) or non-ecogroup connections (76−14=62). Across all months, S. gallolyticus exhibits a high percent degree saturation to ecogroup (red) taxa compared to non-ecogroup taxa (blue). The bar graph of FIG. 28C illustrates that averaging the percent degree saturation over all months for all 76 taxa illustrates that ecogroup taxa (red) preferentially co-vary with each other.

### Example 2

### Development of the Ecogroup Network

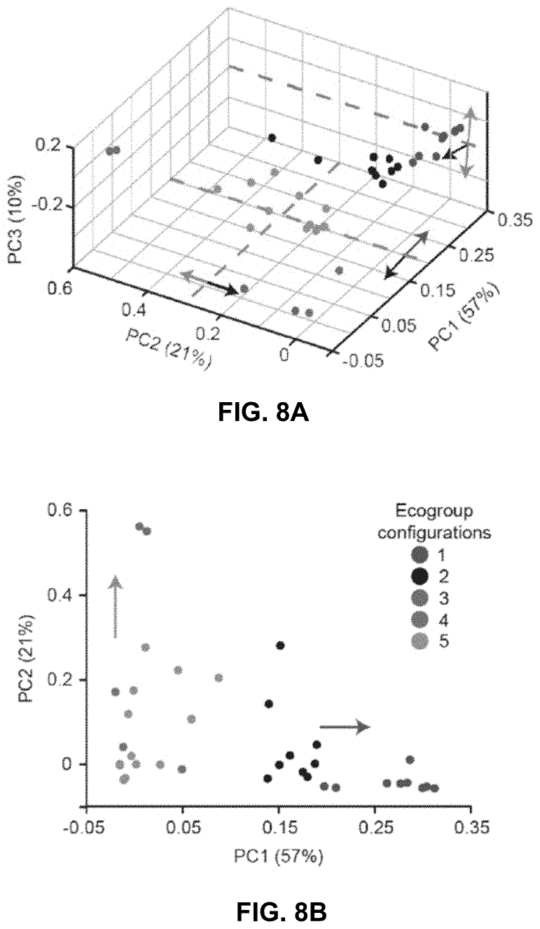

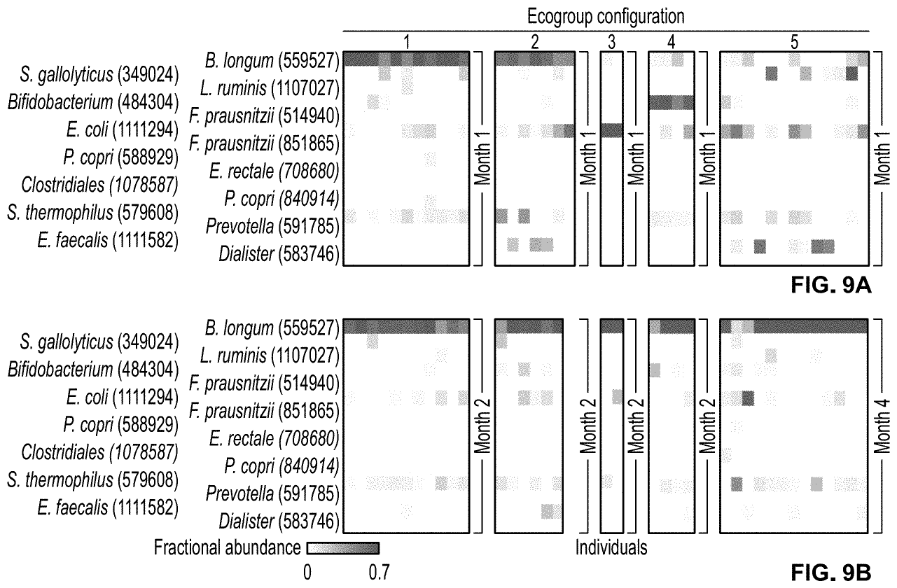

How does the co-varying network develop? To address this question, we first sought a quantitative measure of initial ecogroup structure. A matrix was constructed where each row was a fecal sample collected at postnatal month 1 (n=37) and each column was an ecogroup taxon; each element in the matrix was the fractional abundance of an ecogroup taxon. PCA was performed over the rows of this matrix; plotting each fecal sample onto a space defined by the top three principal components revealed five different groups of fecal samples (FIGS. 8A and 8B). Noting the fractional abundances of ecogroup taxa within each of the five groups of fecal samples revealed five distinct ecogroup taxa profiles, or ‘ecogroup configurations’ at postnatal month 1 (FIGS. 8B and 9A).

FIGS. 8A, 8B, and 9 summarize ecogroup configuration in healthy members of the 5-year Mirpur birth cohort. FIGS. 8A and 8B illustrate the results of PCA, performed on a matrix where each row is a fecal sample obtained at postnatal month 1 and each column is an ecogroup taxon, defines three principal components whose variances are shown in parenthesis. Each fecal sample is plotted. Dashed lines in FIG. 8A represent separation of all fecal samples from postnatal month 1 into 5 groups (colored and labeled 1 through 5). Each group has a specific taxa profile (configuration). FIG. 8B provides another view of subjects at postnatal month 1, with projection onto PC1 and PC2. FIG. 9A illustrates the ecogroup configuration at postnatal month 1. Projection along PC1 in FIG. 8B corresponds to the extent of B. longum predominance, as evidenced by ecogroup configurations 1 and 2. Ecogroup configurations 3, 4, and 5 exhibit an initial mix of other ecogroup taxa. FIG. 9B illustrates ecogroup configurations 1, 2, 3, and 4 at postnatal month 2, and ecogroup configuration 5 at month 4. Note that all configurations converge to a B. longum-predominant state by postnatal month 4.

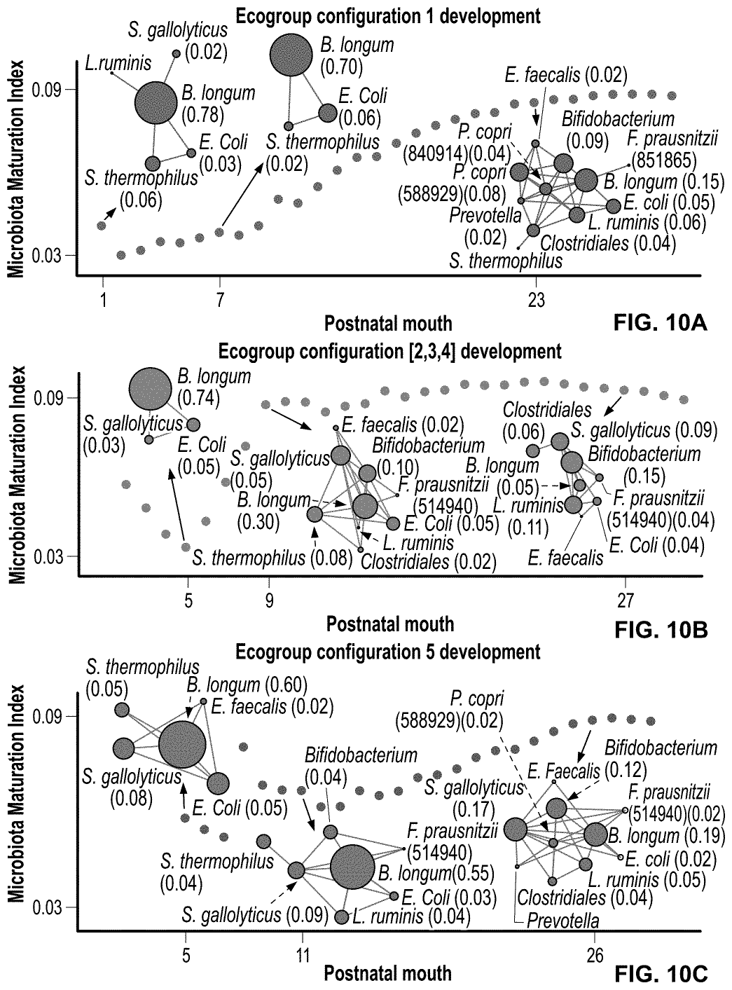

By postnatal month 4, the fractional abundance profile of each ecogroup configuration converged to a B. longum dominant state (FIG. 9B). Because ecogroup configurations 2, 3, and 4 converge to a B. longum predominant state at month 2, we combined individuals comprising configurations 2, 3, and 4 into a single group. Performing iPCA on configurations 1, 2, 3, 4, and 5 allowed us to track the development of each configuration through time. Configurations 1 and 5 mature over the first 20 months while configuration 2, 3, and 4 matures over the first 9 months (FIGS. 10A-C).

FIGS. 10A, 10B, and 10C illustrate the development of ecogroup configurations in the gut microbiota of healthy members of the Mirpur birth cohort, including ecogroup configuration 1 (FIG. 10A), ecogroup configurations 2, 3, and 4 (FIG. 10B), and ecogroup configuration 5 (FIG. 10C), as measured by ‘Microbiota Maturation Index’. The greater the y-axis value, the more dissimilar the ecogroup organization relative to the reference time point (month 1 for configuration 1, month 2 for configurations 2, 3, and 4 and month 5 for configuration 5. Three distinct time points are highlighted to represent the initial B. longum dominant state, an intermediate developmental state, and a mature state. Configurations 1 and 5 are mature by months 23 and 26 while configurations 2, 3, and 4 mature by month 9. Fractional abundance values of ecogroup taxa are noted in parenthesis.

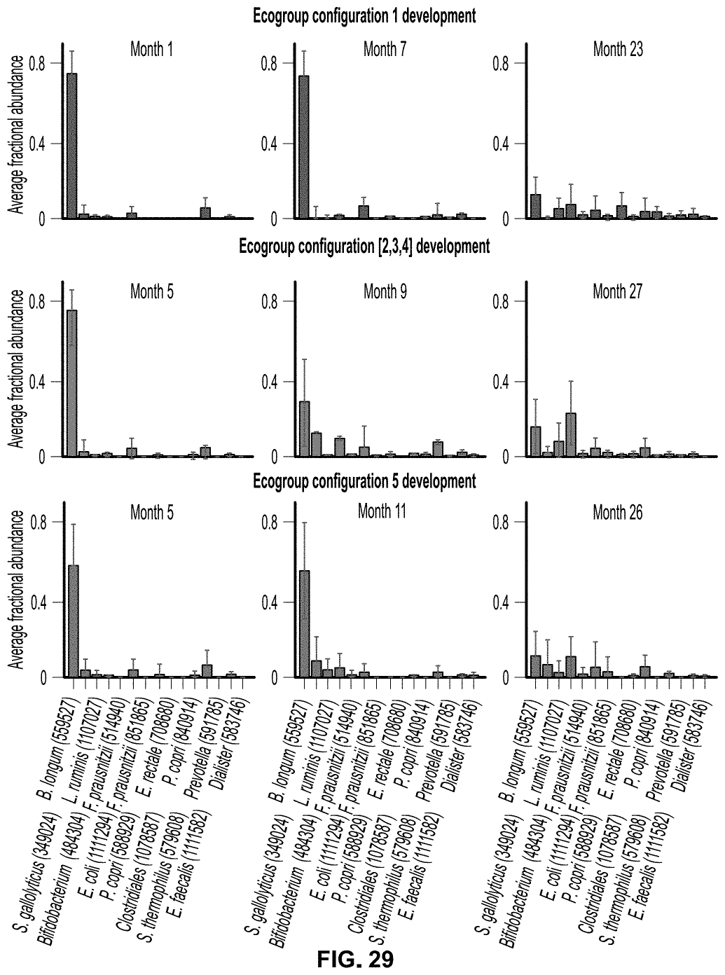

During this time, the fractional abundance of B. longum in each of these configurations decreases while the abundances of other ecogroup taxa (L. ruminis, a Bifidobacterium, S. gallolyticus, E. coli, and Clostridiales) increase (FIG. 29). FIG. 29 illustrates changes in the fractional abundances of ecogroup taxa over time. The fractional abundance distributions of taxa over the first two years of postnatal life for ecogroup configurations 1, 2, 3, 4, and 5. Mean values±SD are shown. All ecogroup configurations show an initial B. longum predominance. As microbiota development proceeds, the fractional abundance of B. longum decreases.

How the ‘organization’ of each configuration progresses can be characterized by considering covariance between ecogroup taxa at each time point shown in FIGS. 10A, 10B, and 10C. At early time points, the organization of each configuration is sparse with a single dominant taxon co-varying with few other taxa; each configuration then assumes a more complex organization characterized by the presence of multiple ecogroup taxa that co-vary. Together, these results reveal a developmental program that is multifactorial, involving differences in ecogroup fractional abundances over time as well as an evolving ecogroup organization.

Varying the 20% co-variance threshold minimally changes ecogroup network structure. Covariance matrices at the time points highlighted in FIG. 10A, 10B, and 10C were calculated. Matrices were binarized according to the top 10, 20, and 25% of taxon-taxon covariance values for ecogroup configurations 1, 2, 3, 4, and 5. For each threshold, the significantly co-varying taxon-taxon pairs retained from the next-most stringent threshold are shown in gray.

### Example 3

### The Generalizability of the Ecogroup to Other Birth Cohorts

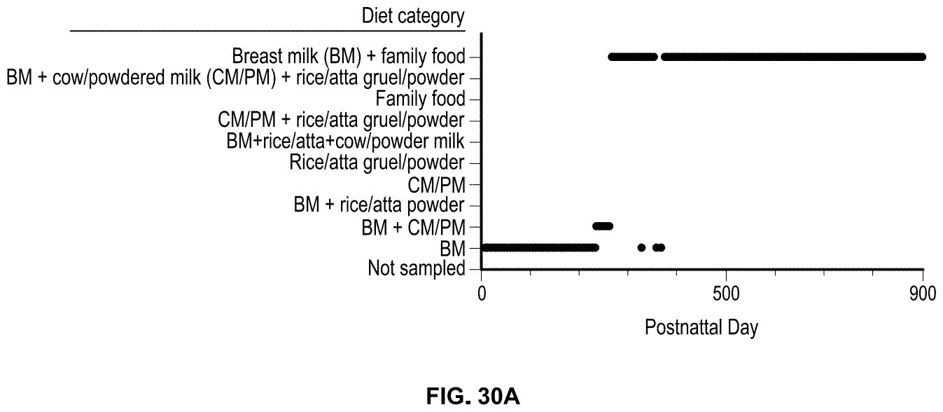

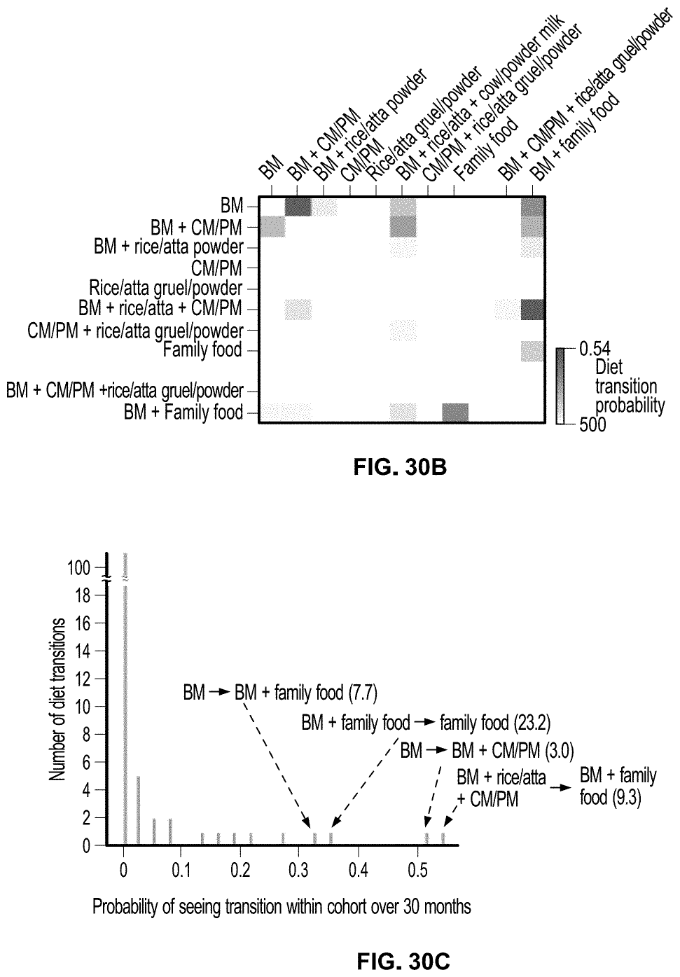

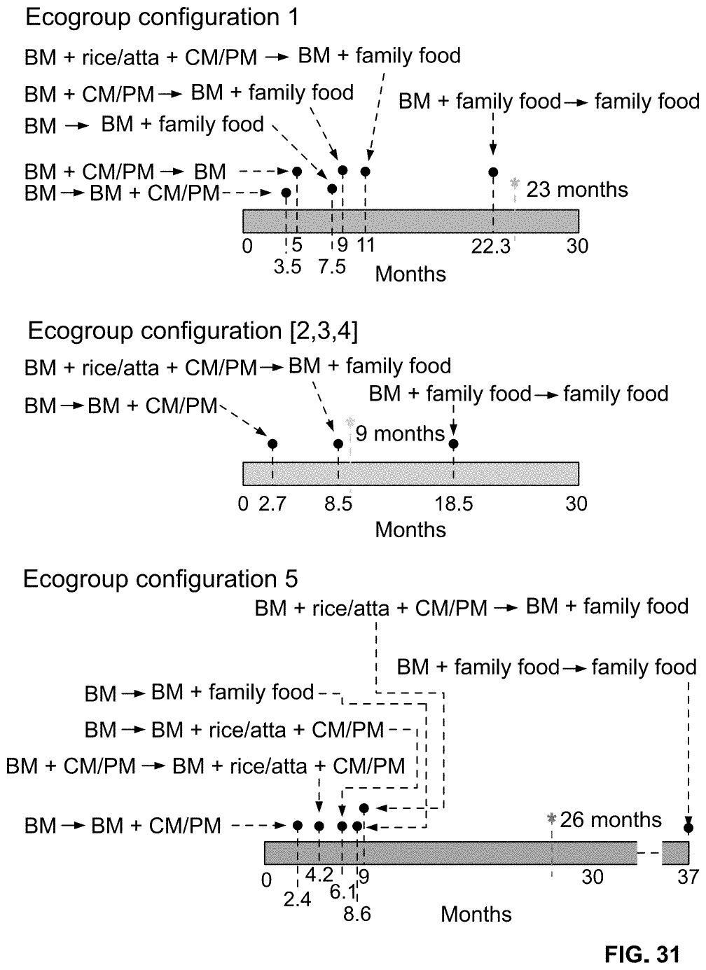

The ecogroup could be a direct result of the particular nature of foods being fed to infants and children within the sampled neighborhood of Dhaka, Bangladesh, or a more conserved feature of gut microbiota organization that exists independent of a child's dietary landscape. We addressed this question by relating the dietary practices of cohort members to their ecogroup development and by examining the ability of ecogroup taxa to describe the developing microbiota of healthy members of birth cohorts residing in other low/middle-income countries. Overall, we found no obvious correlation between dietary transitions and ecogroup development (FIGS. 30A, 30B, 30C, and 31) in this small cohort.

FIGS. 30A, 30B, 30C, and 31 illustrate the effects of dietary transitions and ecogroup configurations in members of the healthy Mirpur cohort. FIG. 30A shows a sample diet profile of a cohort member. Dietary history and transitions can be measured as a function of postnatal day. Diet categories were defined as follows; breast milk (BM) only, BM plus cow's milk or reconstituted powdered bovine milk (CM/PM), BM plus Rice/Atta (whole meal wheat flour) powder, CM/PM only, Rice/Atta gruel/powder, BM plus Rice/Atta plus CM/PM, CM/PM plus Rice/Atta gruel/powder, other Family Food, BM plus CM/PM plus Rice/Atta gruel/powder, or BM plus other Family Food. FIG. 30B shows a ‘transition’ matrix where each pixel represents the probability of seeing a transition to a new diet category from the row to the column. Because of the hysteretic nature of dietary history, this transition matrix is not symmetric. FIG. 30C is a histogram of all pixels in the matrix shown in FIG. 30B) showing that only a few diet transitions are observed with a high probability. Numbers in parenthesis after each listed category represent the average month of dietary transition. FIG. 31 shows dietary transitions for individuals with microbiota harboring each ecogroup configuration. The dashed vertical line with an asterisk indicates the month at which the ecogroup configuration reaches maturity.

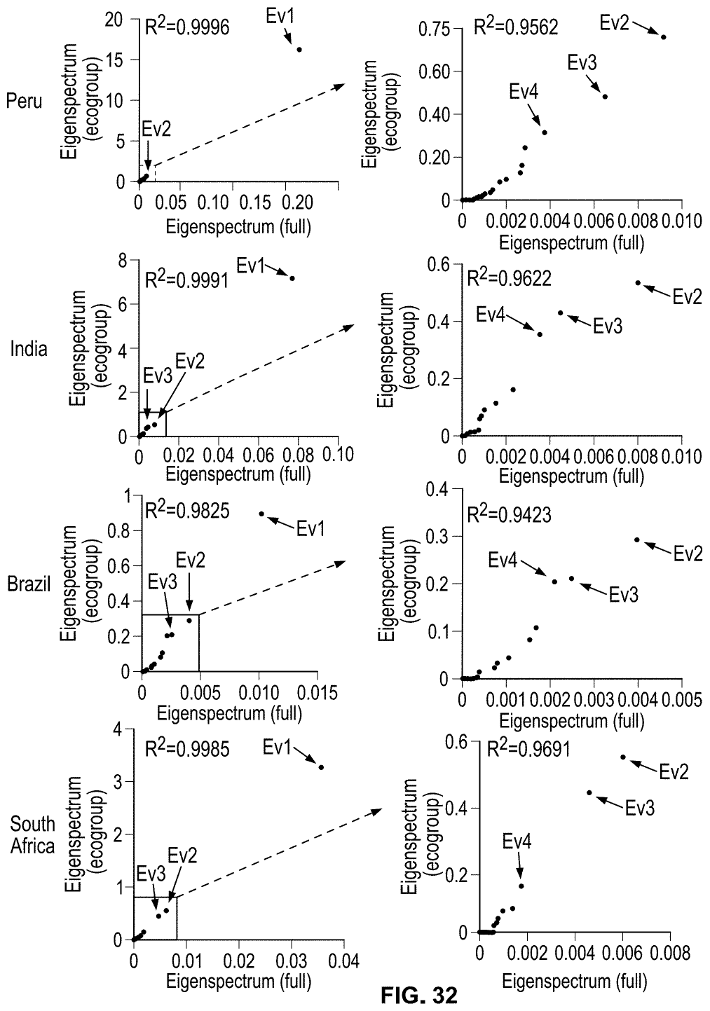

To determine the extent to which the ecogroup is a generalizable descriptor of the microbiota in infants and children with healthy growth phenotypes, we turned to the MAL-ED network of study sites located in low- and middle-income countries. Fecal samples had been collected monthly for the first 2 postnatal years from healthy members of birth cohorts residing in Loreto, Peru (pen-urban area), Vellore, India (urban), Fortaleza, Brazil (urban), and Venda, South Africa (rural). Our ability to identify a network of co-varying taxa in the Mirpur cohort depended on a high-resolution time series study that extended well beyond the month at which the microbiota was determined to be ‘stable’ (month 21). This duration of sampling did not occur at these other sites, making it difficult to identify conserved covariance among taxa within their mature gut communities. However, we were able to test how well the 15 ecogroup taxa identified in the Mirpur cohort could characterize the microbiota of individuals living elsewhere. To do so, we computed the eigenspectrum of fecal samples obtained from each country using either the complete list of OTUs identified in the fecal communities of all cohort members from all countries (n=1459 taxa satisfying the criteria of having fractional relative abundance >0.001 in at least 2 samples), or the 15 ecogroup taxa identified from the Mirpur cohort. If the ecogroup taxa were a good representation of the full microbiota, the ecogroup should capture the variance between fecal samples to a similar degree as the complete OTU list; FIG. 32 demonstrates that this is the case.

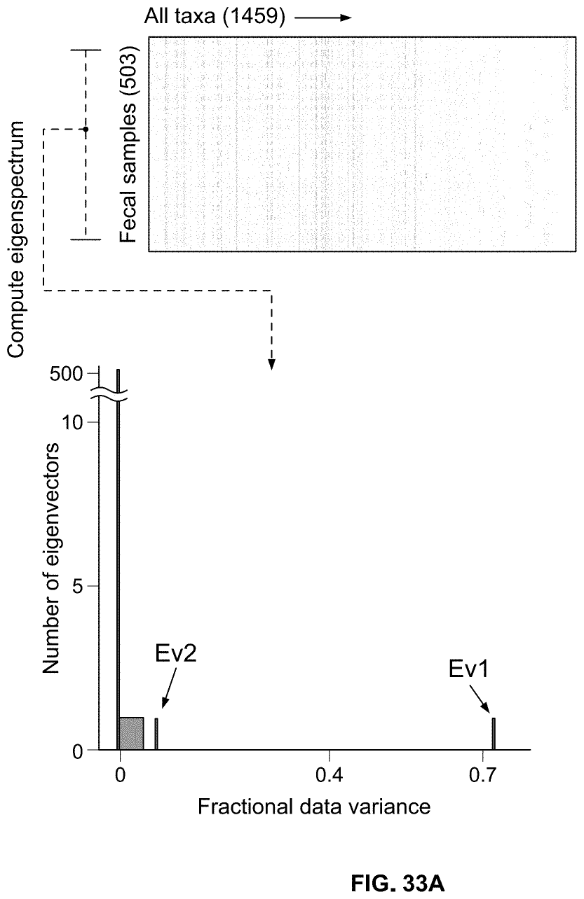

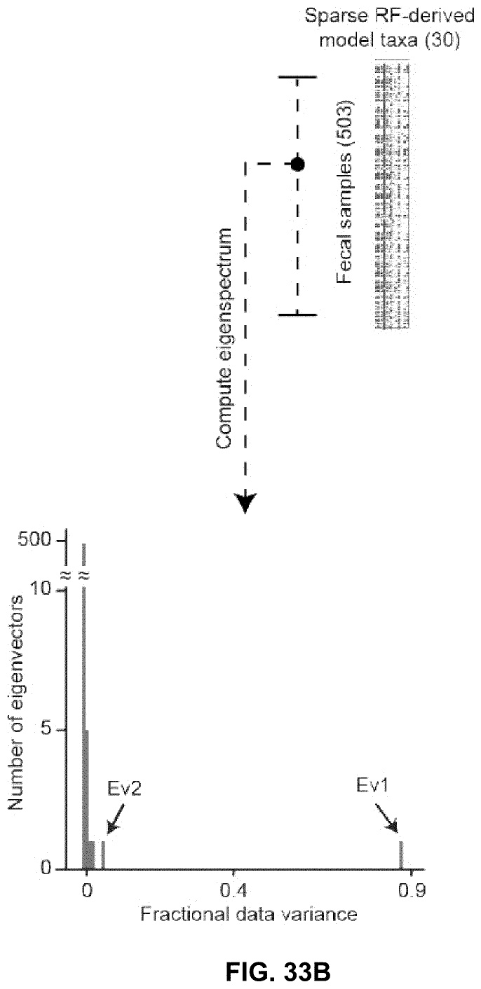

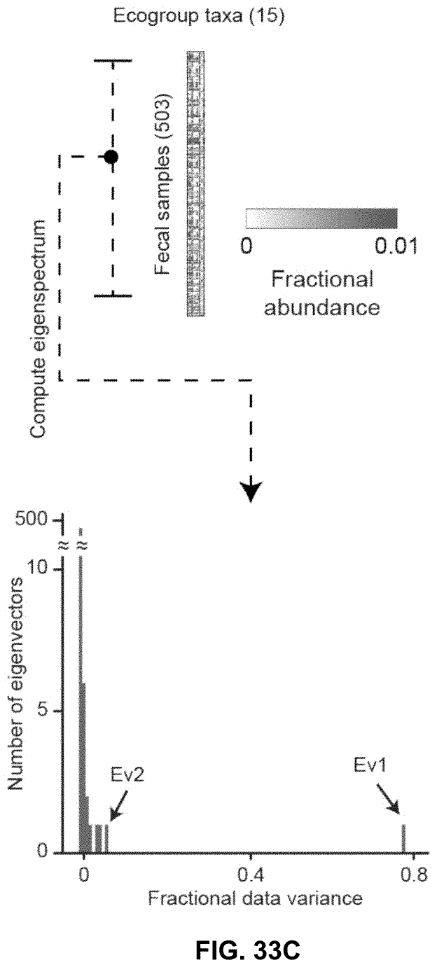

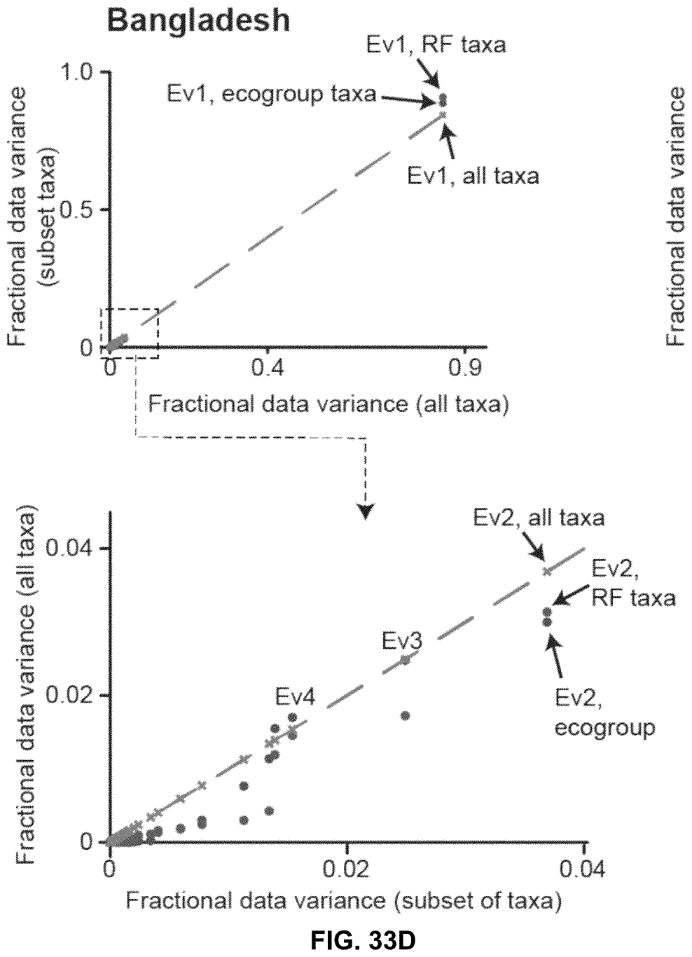

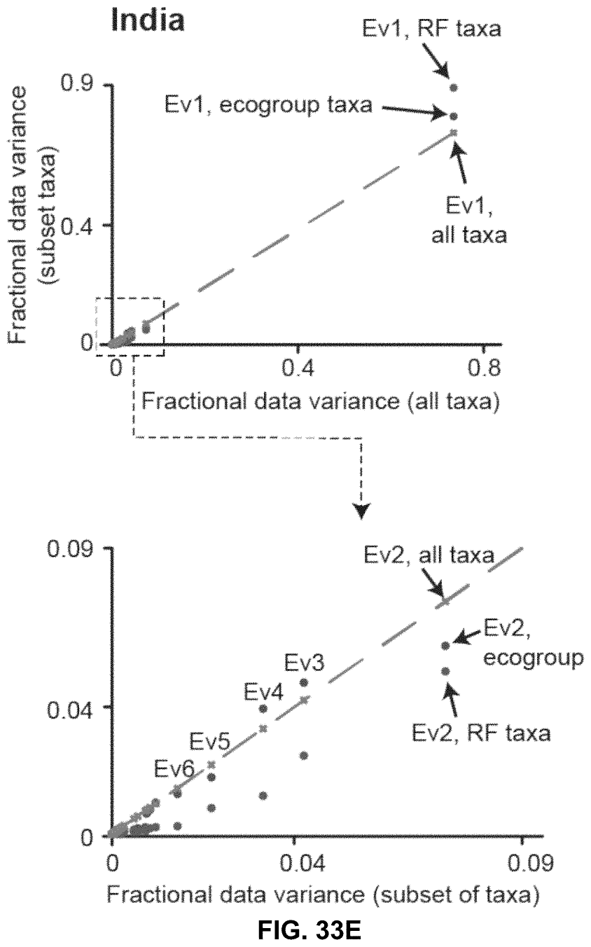

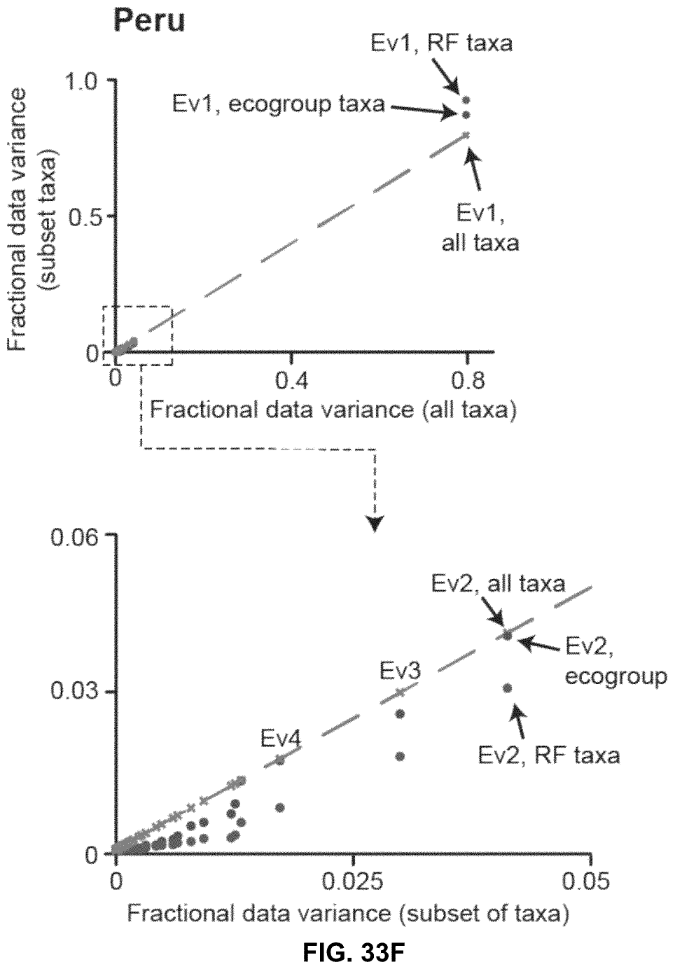

FIG. 32 illustrates that the 15 ecogroup taxa identified in the Mirpur cohort represent a generalizable and marked dimension reduction for describing gut microbiota form in healthy members of birth cohorts representing diverse geographic locales. An eigenspectra were computed (spectrum of eigenvectors (Ev)) using the 15 bacterial OTUs in the Mirpur ecogroup, and plotted against eigenspectra computed using all 1459 OTUs identified across all monthly fecal samples within and across the indicated geographic sites. The results for each birth cohort are displayed, together with the corresponding r2 values (Pearson) between the eigenspectra generated using the ecogroup taxa or all OTUs. The 15 ecogroup taxa were compared to the 30 taxa generated from the sparse 2-year RF-derived model of gut microbiota development in healthy members of Bangladeshi, Peruvian, and Indian birth cohorts. As an example of the computational workflow, eigenspectra are computed over all fecal samples from the Indian cohort (n=503 fecal samples) using either (i) all taxa (FIG. 33A), (ii) taxa in the sparse Indian RF-derived model (FIG. 33B), or (iii) the ecogroup taxa (FIG. 33C). As illustrated in FIGS. 33D-33F, one eigenvector (Ev1) carries most of the variance over fecal samples for each matrix, with many lower order eigenvectors (e.g., Ev2) carrying less variance. The fractional variance of each eigenvector computed by considering all taxa (x-axis) is plotted against the fractional variance of each eigenvector computed by considering a subset of taxa (red dots for RF-derived model taxa, blue dots for ecogroup taxa). The dashed line represents a perfect recreation of the eigenspectrum from the full list of taxa. The closer an eigenvector is to the dashed line, the more precisely it captures the variance of fecal microbiota samples defined by all taxa. A zoom-in of the dashed box shown in each plot focuses on lower order eigenvectors.

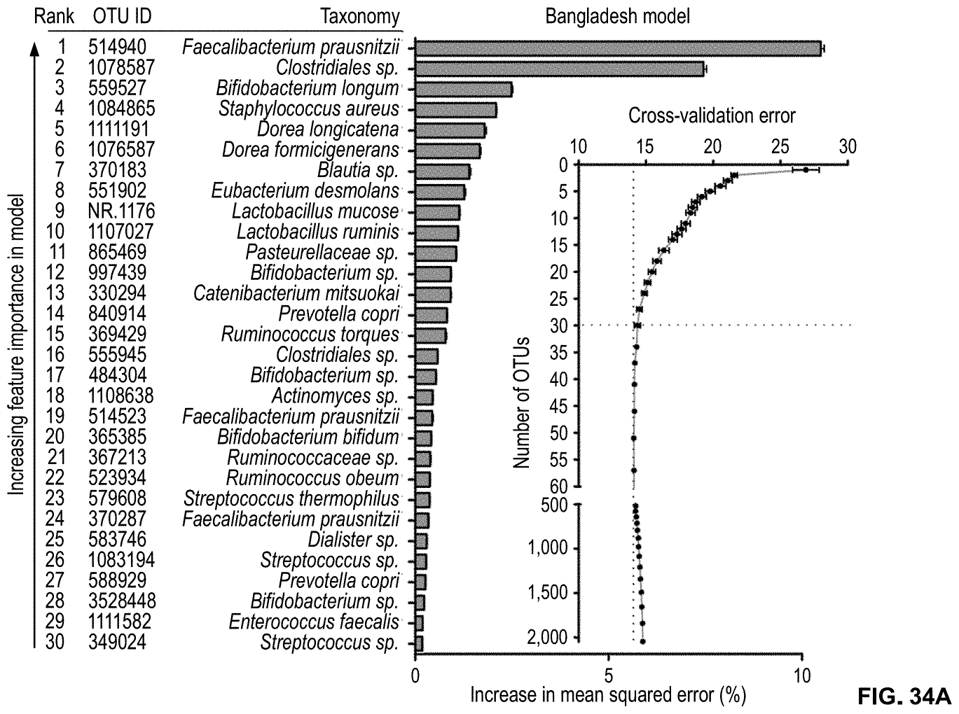

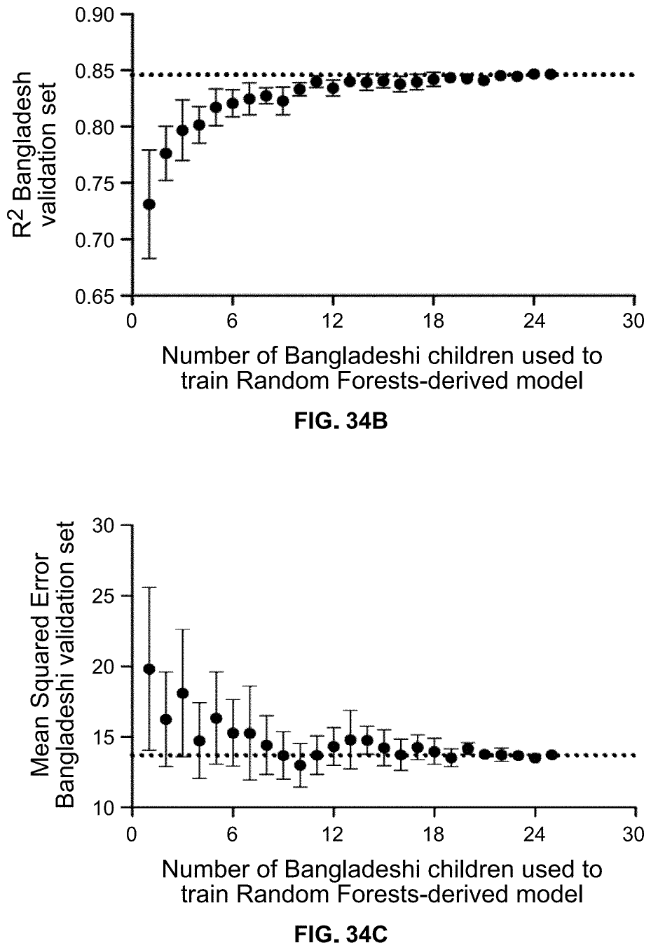

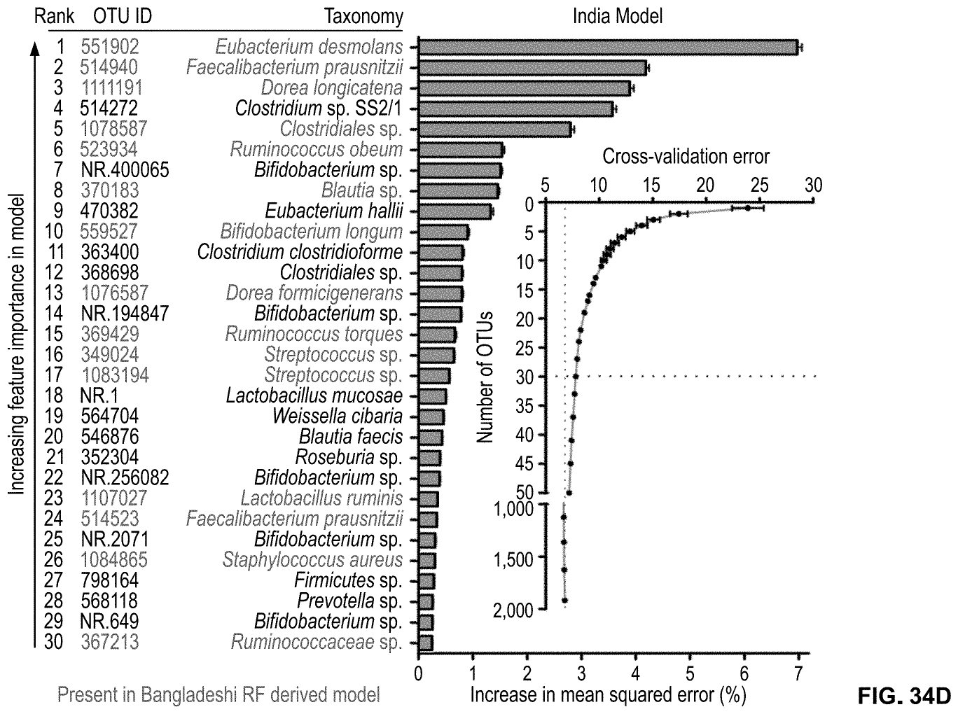

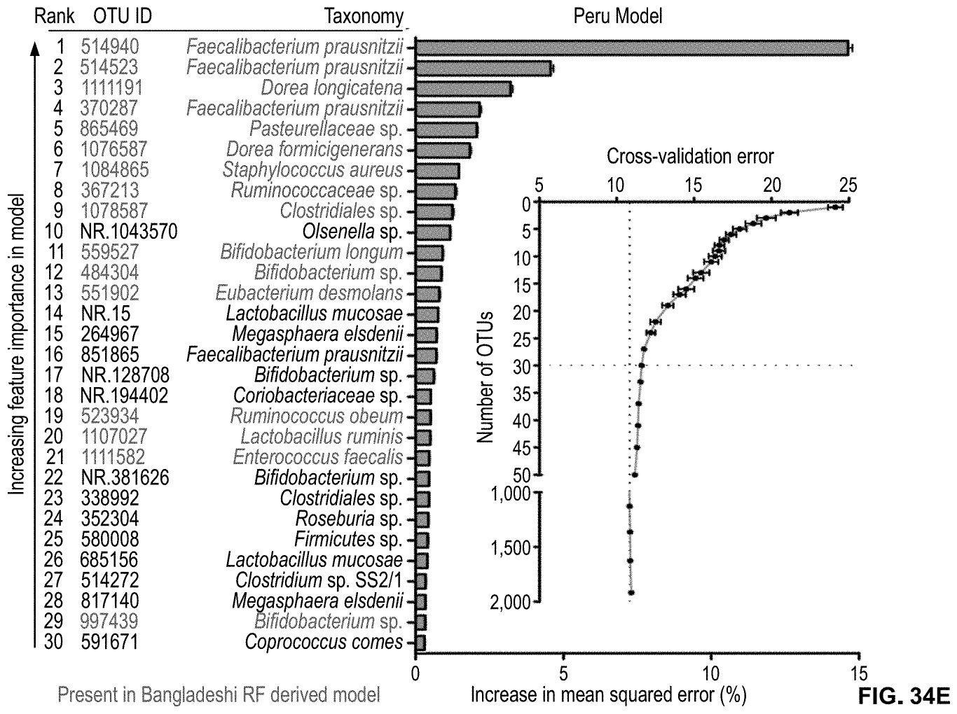

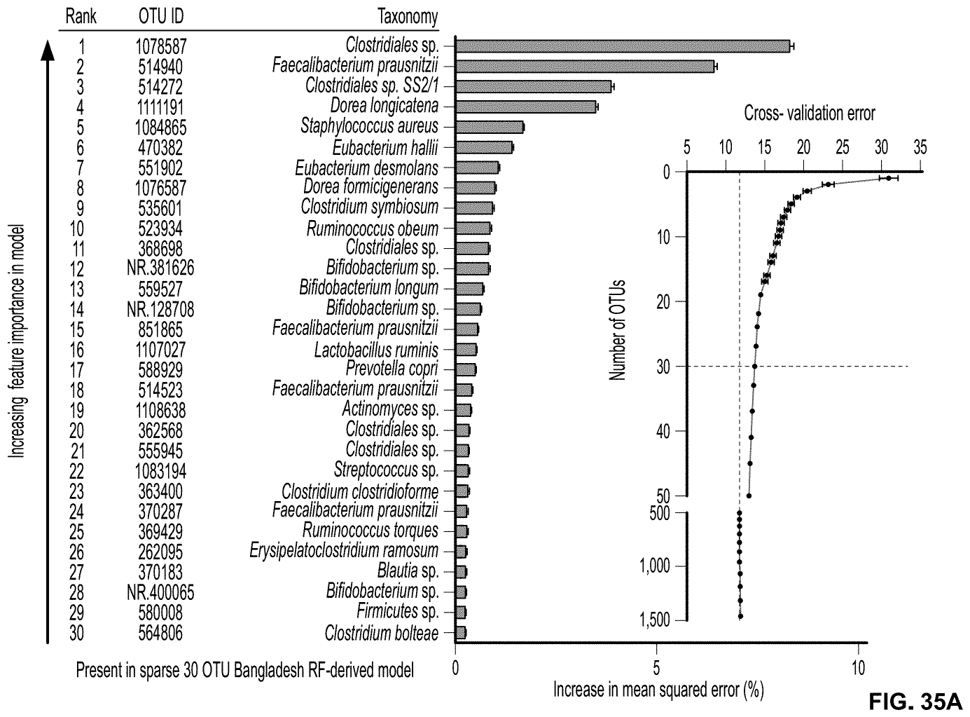

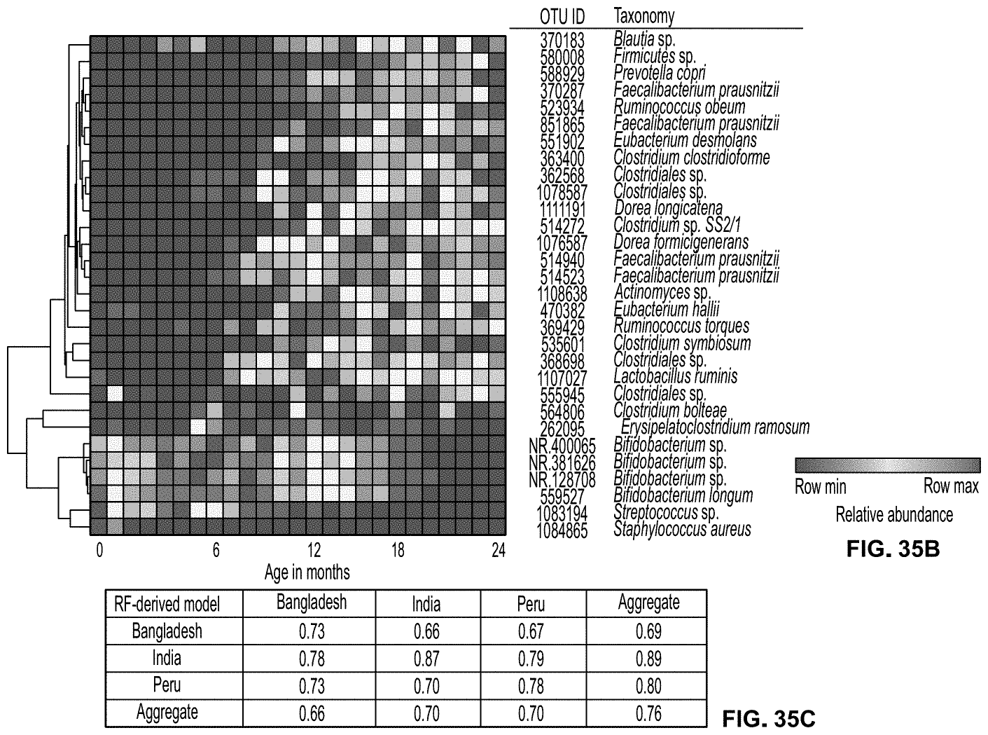

We directly compared the sufficiency of the top 30 age-discriminatory taxa in sparse RF-generated models of normal microbiota development generated from Peruvian, Indian, and Bangladeshi birth cohorts (FIG. 34-35), versus the 15 ecogroup taxa identified through statistical analysis of only the Bangladeshi cohort, to recapture fecal sample variance in each population (FIG. 33A). We found that across the different geographies, the 15 ecogroup taxa more precisely recreates the eigenspectrum, particularly at lower principal components (FIGS. 33C-E). We concluded that the ecogroup represents a conserved structural feature of a developing gut microbiota of humans living in these different locations with diverse cultural traditions.

FIGS. 34-35 illustrate Random Forests (RF)-derived sparse 2-year models of gut microbiota development in healthy members of birth cohorts from Bangladesh, India and Peru. FIG. 34A shows a sparse 30 OTU RF-derived model generated from healthy members of the Mirpur birth cohort (n=25 individuals; 539 fecal samples) in which OTUs are ranked in descending order of their importance to the accuracy of the model. The x-axis plots the increase in mean-squared error when abundance values from each OTU are randomly permuted. The inset shows the cross-validation curves that result from reducing the number of 97% ID OTUs used for model training. FIGS. 34B and 34C illustrate the sample size estimation for RF-derived model training. Subsampling of the training set of healthy Bangladeshi children (n=25) was performed and validated on a separate set of 25 children in this 2-year birth cohort study. As the number of children incorporated into a model is reduced, there is a reduction in Pearson's correlation coefficient (FIG. 34B) and an increase in the mean squared error rate (FIG. 34C). These effects plateau when >12 children are included in the model training. FIGS. 34D and 34E illustrate sparse RF-derived models generated from members of birth cohorts, sampled monthly, living in Vellore, India (331 fecal samples from 14 individuals (FIG. 34D) and Loreto, Peru (505 fecal samples from 22 individuals; FIG. 34E).

FIG. 35A illustrates an ‘Aggregate’ model generated by combining V4-16S rDNA datasets generated from monthly fecal samples collected during the first 2 years of postnatal life from members of the Peruvian, Indian, Brazilian and South Africa birth cohorts. FIG. 35B is a heat map showing temporal changes in the mean relative abundances of age-discriminatory OTUs comprising the sparse ‘aggregate’ RF-derived model. FIG. 35C is a table summarizing reciprocal tests of the various RF-derived models of gut microbiota development. R² values shown for the Pearson correlation between microbiota age and chronological age were calculated using the indicated RF-derived model and birth cohort.

### Example 4

### Ecogroup Configuration in Moderate Acute Malnutrition

Bangladeshi children with acute malnutrition have perturbed microbiota development; their gut communities appear younger than those of chronologically age-matched healthy individuals. As a first step in testing whether the 15 ecogroup taxa can be used to classify microbiota in undernourished children, we turned to a separate cohort of sixty-three 12- to 18-month-old children from Mirpur diagnosed with moderate acute malnutrition (MAM) who were enrolled in double-blind, randomized controlled trial of four different supplementary foods. Fecal samples were collected for 9 weeks at weekly intervals. The first two weeks comprised a pre-treatment observation period. Over the next 4 weeks, children received either one of three microbiota-directed complementary foods (MDCFs), or a ready-to-use supplementary food (RUSF) representing a form of conventional therapy that, unlike the MDCFs, was not designed to target specific members of the gut microbiota and repair community immaturity. The last two weeks represented the post-treatment observation period. In total, we identified 945 97% ID OTUs that had a fractional abundance of at least 0.001 in at least one fecal sample collected from one or more participants prior to, during and following treatment (n=531 samples). Fecal samples from 30 healthy children, spanning 10-25 months of age, were used as controls (1 sample/individual; note that this group of Mirpur children was not the same as the healthy individuals described above and therefore could be used as an independent validation of healthy ecogroup maturation).

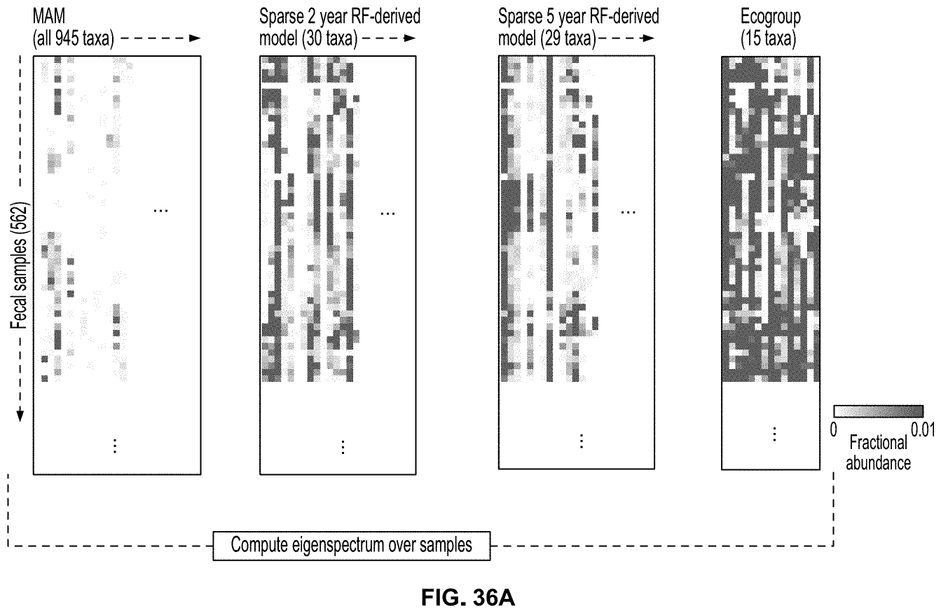

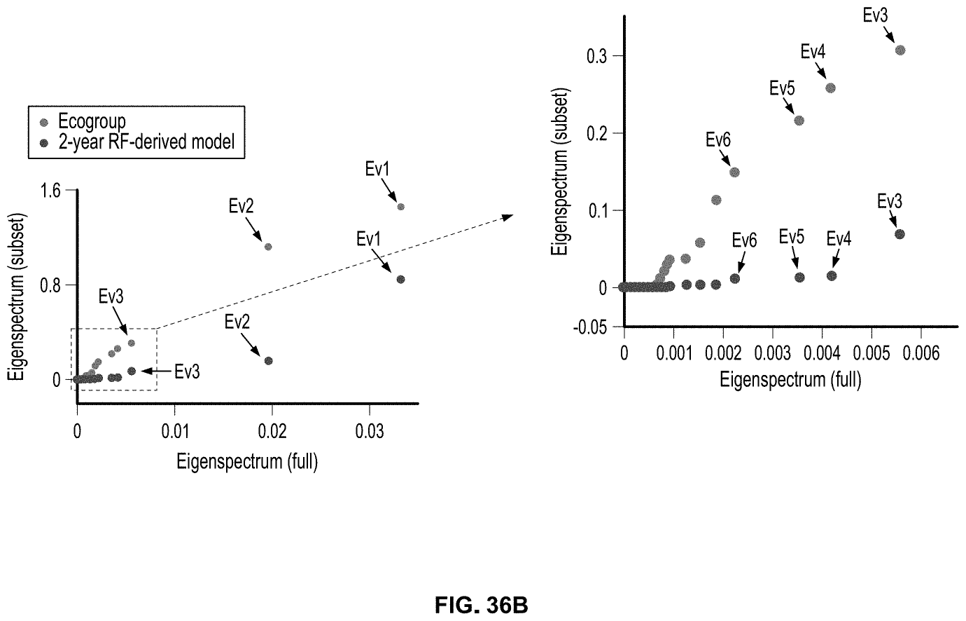

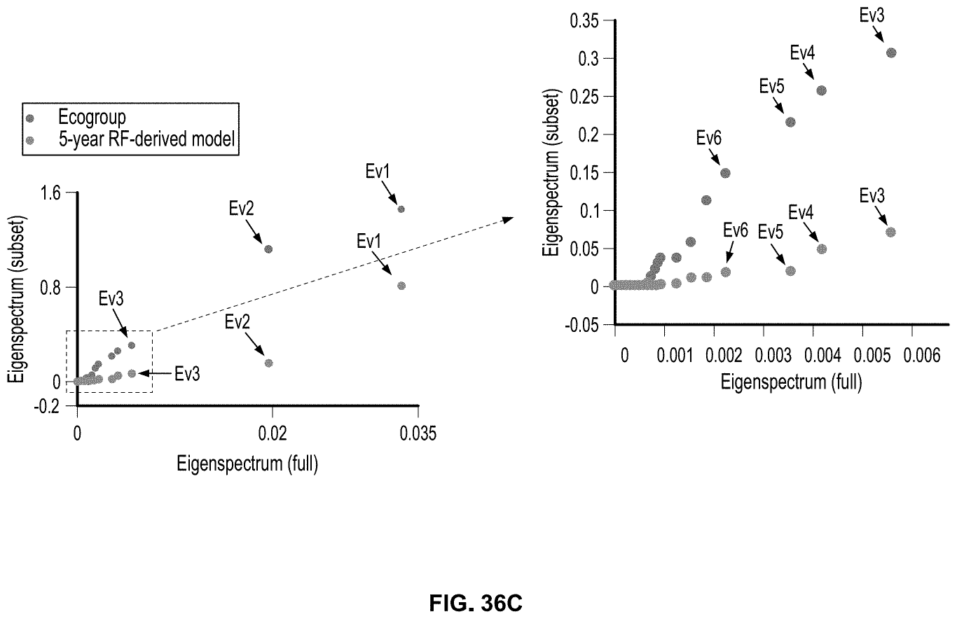

We compared the variance that separates all MAM samples plus samples from the healthy controls using (i) all 945 taxa, (ii) 30 taxa in a sparse RF-derived model of healthy microbiota development generated from members of a Mirpur cohort during the first 2 years of life, (iii) the 29 taxa in the sparse RF-derived model trained on the 5-year dataset (see above), and (iv) the 15 ecogroup taxa (FIG. 36). Comparing the eigenspectra for (i) through (iv) disclosed that PC1 generated from the taxa from either of the sparse RF-generated models or the ecogroup capture gross sample variance. Moreover, the ecogroup taxa capture the full microbiota eigenspectrum at markedly lower principal components (FIG. 36B).

FIGS. 36A, 36B, and 36C present evidence that the ecogroup captures information that specifies microbiota state in the MAM cohort. FIG. 36A is a heat map of fractional abundances of indicated groups of taxa (columns) measured over all fecal samples collected from 63 members of the MAM cohort (rows) (n=562 fecal samples). FIG. 36B shows eigenspectra (spectrum of eigenvectors (Ev)) computed from taxa in the ecogroup, and from the 2-year and 5-year RF-derived models of gut microbiota development, plotted against the eigenspectrum computed from all 945 bacterial OTUs. As illustrated in FIG. 36C the eigenspectrum generated by the 15 ecogroup taxa recreates variance at lower principal components than taxa comprising the RF-generated models, demonstrating that the ecogroup almost entirely captures the information contained within the full list of 945 taxa.

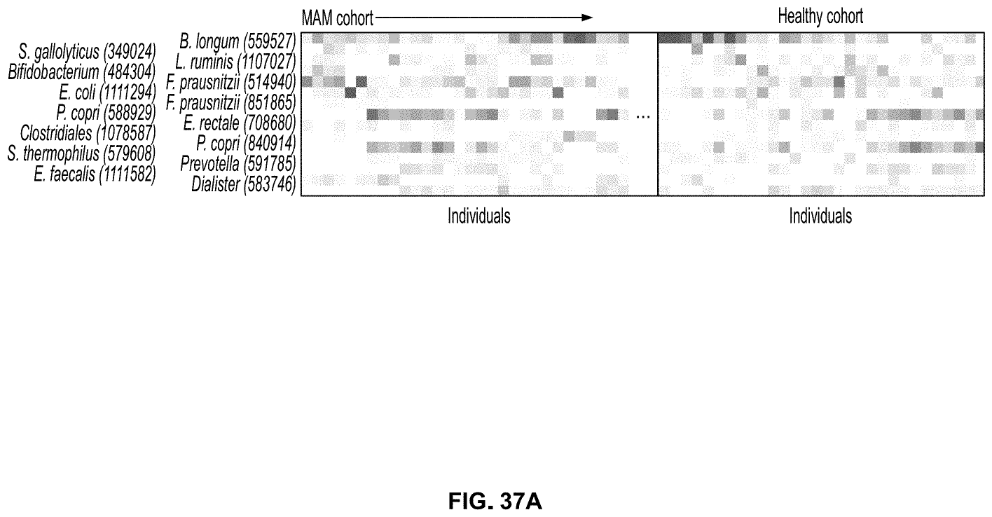

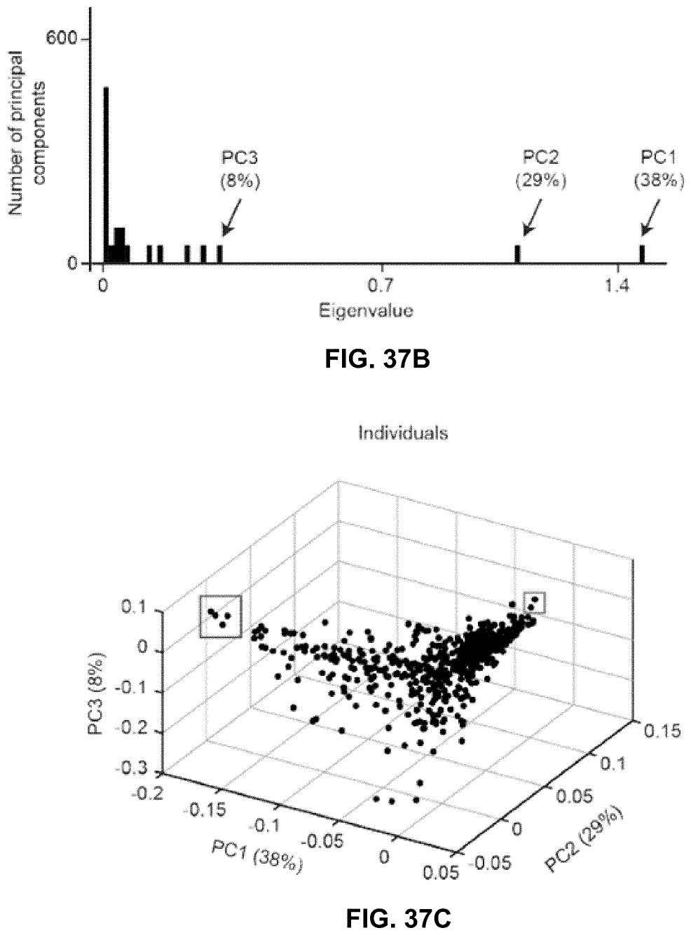

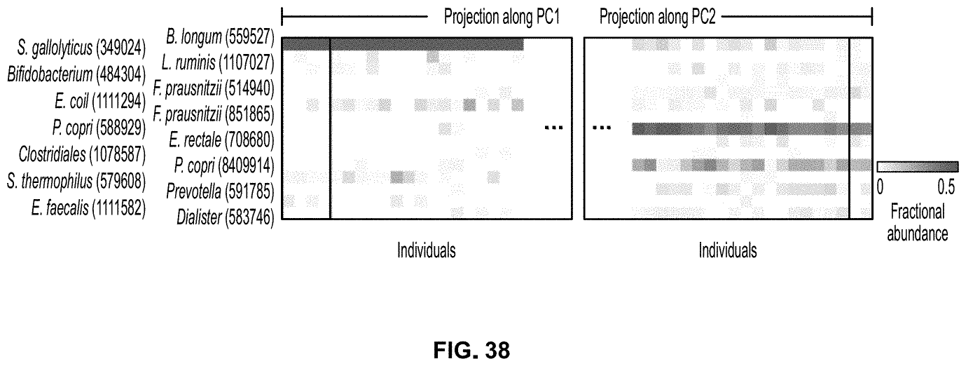

FIGS. 37A, 37B, and 37C illustrate the positioning of MAM and healthy donor microbiota defined by three principal components. FIG. 37A shows a heat map of ecogroup taxa (rows) identified in fecal samples (columns) obtained from children with MAM prior to, during and after treatment with RUSF or a MDCF (n=532 samples), plus those obtained from age-matched healthy controls (n=30 samples), combined into a single dataset to create an ecogroup taxa by fecal sample matrix. Not all MAM samples are shown; the set of dots (ellipsis) represents samples that are not depicted. The matrix in FIG. 37A was decomposed to produce spectrum of principal components (see FIG. 37B). The first three principal components account for 38%, 29%, and 8% of the variance, respectively. FIG. 37C is a projection of the data from individuals in FIG. 37A onto principal component axes; each dot represents a fecal sample from a child with MAM obtained prior to, during or after treatment, or a sample obtained from one of the 30 healthy control subjects. The proximity between points is a quantitative measure of the similarity in fractional abundances of ecogroup taxa between fecal samples. FIG. 38 is a heat map of fractional abundances of taxa in samples from FIG. 37A, ordered by projection onto PC1 and PC2. PCI and PC2 distinguish a B. longum (OTU 559527)-predominant and a P. copri (OTU 840194 and 588929)-predominant ecogroup configuration, respectively (highlighted by the blue and green boxed points in FIG. 37C corresponding to the blue and green boxed columns in FIG. 38).

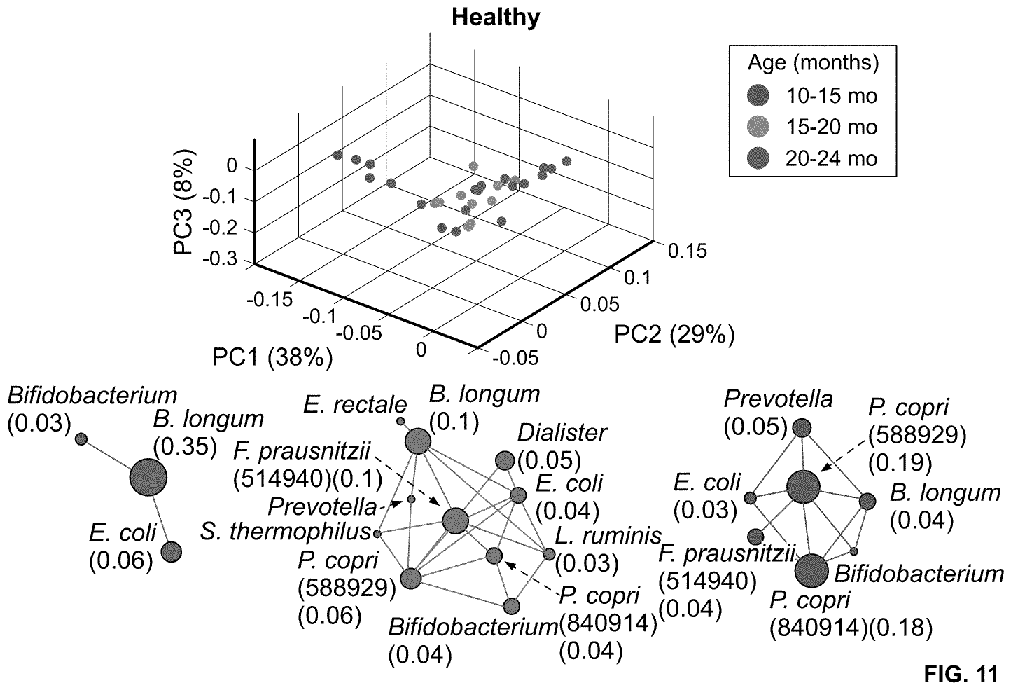

The healthy cohort of 30 subjects was binned into 10 to 15, 15 to 20, and 20 to 25 month age ranges. Projecting these subjects onto PCA space defined by ecogroup taxa revealed a separation of microbiota configurations by age (see FIG. 11 which was constructed using the results shown in FIG. 37A and 37B). FIG. 11 illustrates ecogroup configurations in healthy children. Healthy Mirpur children were grouped by chronologic age, with 10-15-month-old individuals projecting onto PC1, 15-20-monthold subjects centered at the vertex, and the 20-25-month-old group projecting onto PC2. The 10-15- month ecogroup configuration (blue) is comprised of a B. longum predominant, sparse network of covarying taxa whereas the 15-20 month (green) and 20-25 month (red) ecogroup configurations resemble a more complex network organization.

As with the other Mirpur cohort of healthy subjects who had been sampled monthly for 5 years, healthy subjects aged 10-15 months had a B. longum dominant ecogroup network containing a small number of co-varying taxa while those who were 15-25 months old possessed a more taxonomically diverse network.

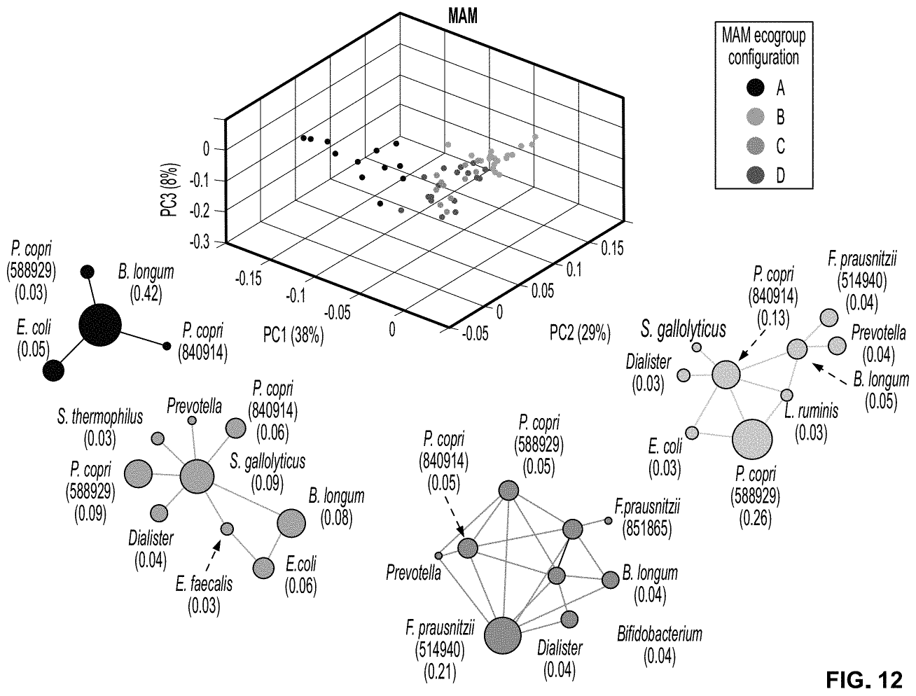

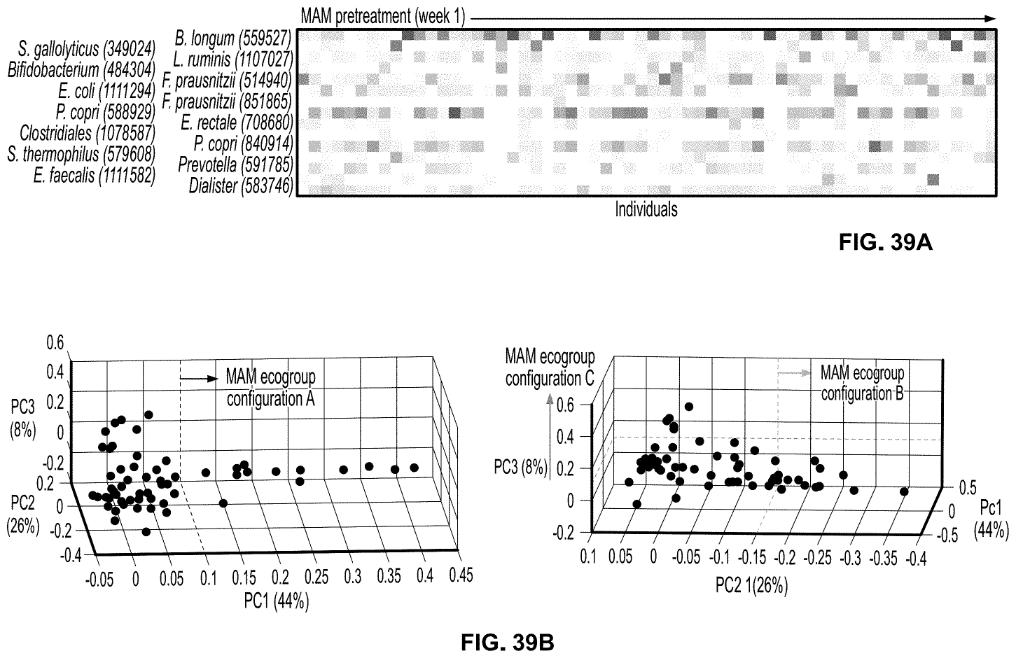

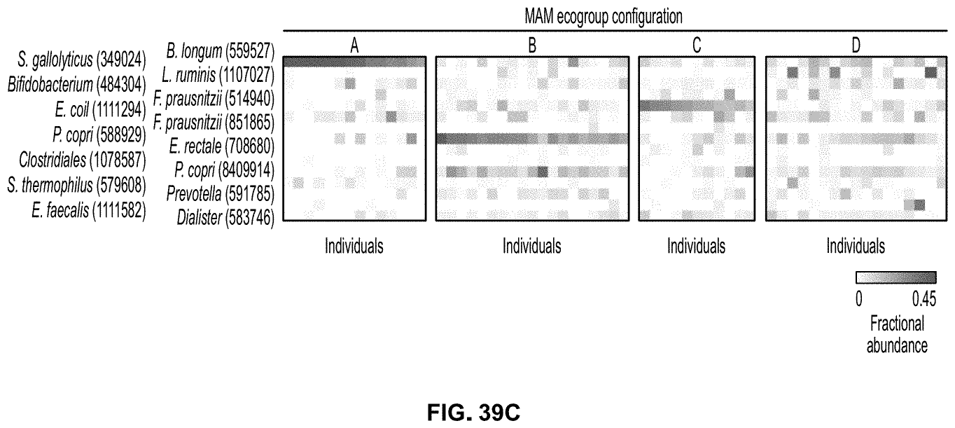

We next characterized ecogroup configuration in the microbiota of children with MAM prior to treatment. Four distinct network configurations were identified. MAM ecogroup configuration A overlaps with the healthy 10-15 postnatal month age ecogroup configuration whereas MAM ecogroup configurations B, C, and D overlap with the ecogroup configurations in healthy 15-25 -month-old individuals (FIG. 12 and FIG. 39).

FIGS. 39A, 39B, and 39C illustrate a process of defining MAM ecogroup configurations. FIG. 39A is a heat map of fractional abundances of ecogroup taxa in the fecal microbiota of all 63 MAM cohort members at week 1 of the trial (pre-treatment). FIG. 39B is a PCA of data from FIG. 39A. Each point represents a single individual from FIG. 39A. The proximity between two points is indicative of the similarity in fractional abundances of ecogroup taxa between fecal samples. Applying the indicated thresholds on each axis defines distinct MAM ecogroup configurations. FIG. 39C demonstrates that B. longum, P. copri (840194 and 588929) and F. prausnitzii (OTU 514940) are the predominant taxa in ecogroup configurations A, B and C, respectively, while configuration D has a more balanced mixture of ecogroup taxa.

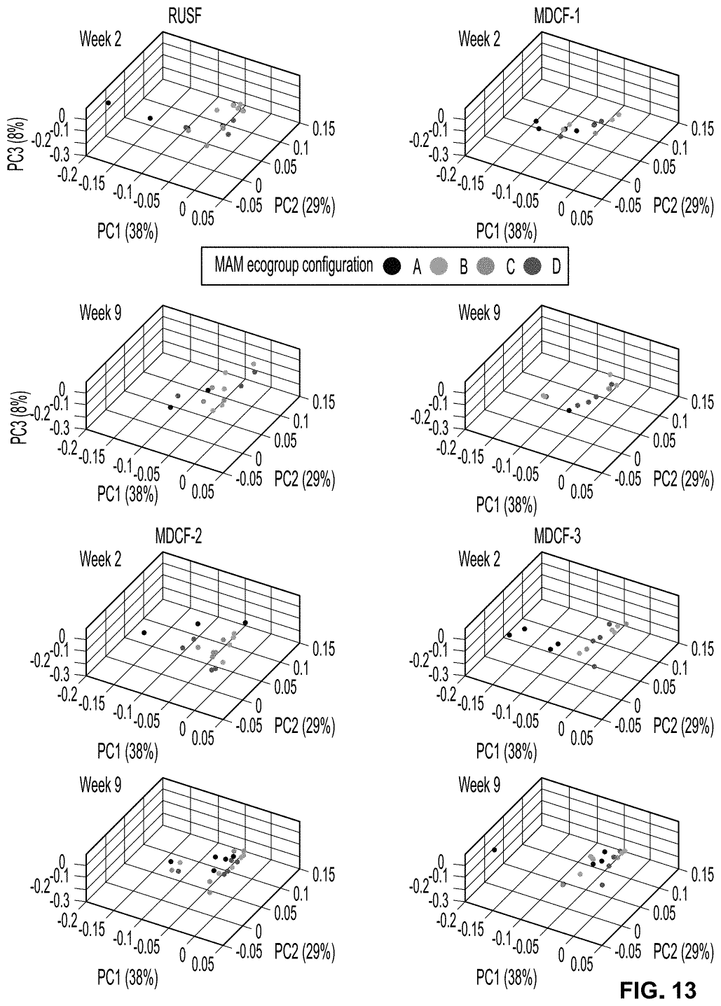

Each treatment arm was composed of 14-17 randomly assigned children; the four MAM ecogroup configurations were represented in each treatment arm in roughly equal proportions, with a given individual possessing a profile of ecogroup taxonomic abundances that belong to one configuration (FIG. 13). Thus, any observed MDCF effect could not be simply ascribed to preferential enrichment of a starting MAM ecogroup configuration within a treatment arm. FIG. 12 illustrates the ecogroup configurations children with MAM. The most immature MAM ecogroup configuration is B. longum predominant with sparse covariance and projects exclusively on PC1. The more mature MAM ecogroup configurations (labeled ‘B’, ‘C’, and ‘D’ in the color code) show a more densely co-varying network structure and project exclusively on PC2 (also see FIG. 39C).

Comparing the PCA plots in FIGS. 11 and 12, and noting the fractional abundances of ecogroup taxa for individuals projected along PC1 and PC2 in FIGS. 37C and 38 reveals that projection along PC1 corresponds to a more immature microbiota configuration (high B. longum fractional abundance with a sparse group of co-varying ecogroup taxa), whereas projection onto PC2 represents a more mature microbiota configuration (more distributed fractional abundance with a more complex pattern of covariance among ecogroup taxa).

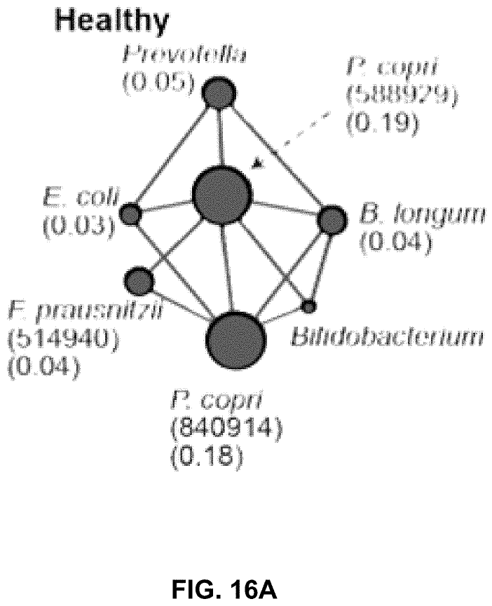

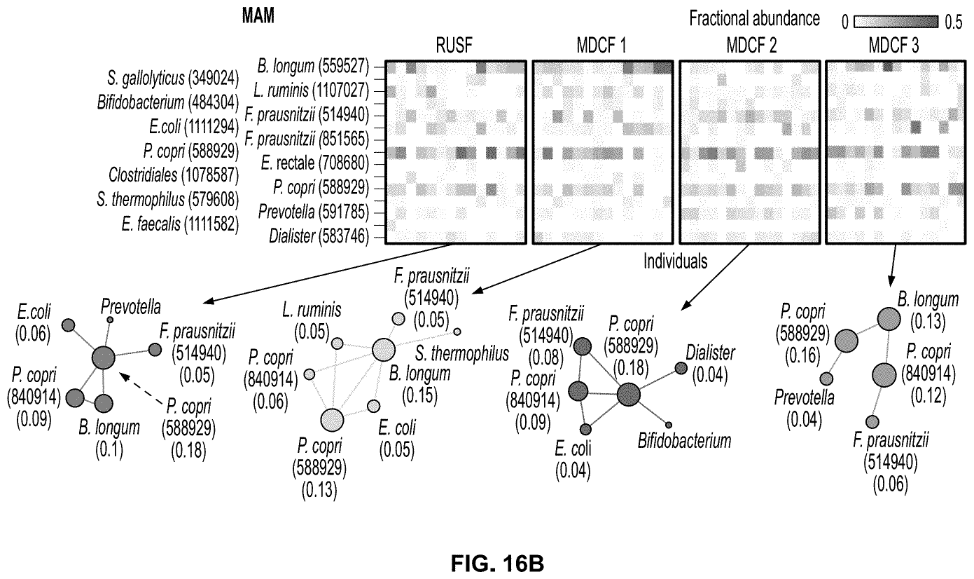

We used changes in projection onto PC1 and PC2 as a metric to assess the efficacy of the different MDCFs in advancing the state of microbiota maturation. We compared the 2- and 9-week time points; i.e., just before and 2 weeks after the intervention. At week 9, network structure in the microbiota of individuals treated with RUSF, MDCF-1 and MDCF-3 exhibited decreased B. longum fractional abundance, and increased covariance of P. copri with other ecogroup taxa. In contrast, MDCF-2 was unique among the MDCFs in producing an ecogroup configuration indicative of a more mature microbiota that lacks any taxa covariance with B. longum (FIG. 16). Notably, MDCF-2 was also distinct among the four treatments types in eliciting changes in the plasma proteome indicative of improved health status including, for example, changes in biomarkers and mediators of metabolism, bone growth, CNS development and immune function. We concluded that the ecogroup descriptor of community organization can be used to compare the effects of microbiota-directed therapeutic interventions. To explore the generalizability of this idea, we turned to a cohort of undernourished children with more severe disease.

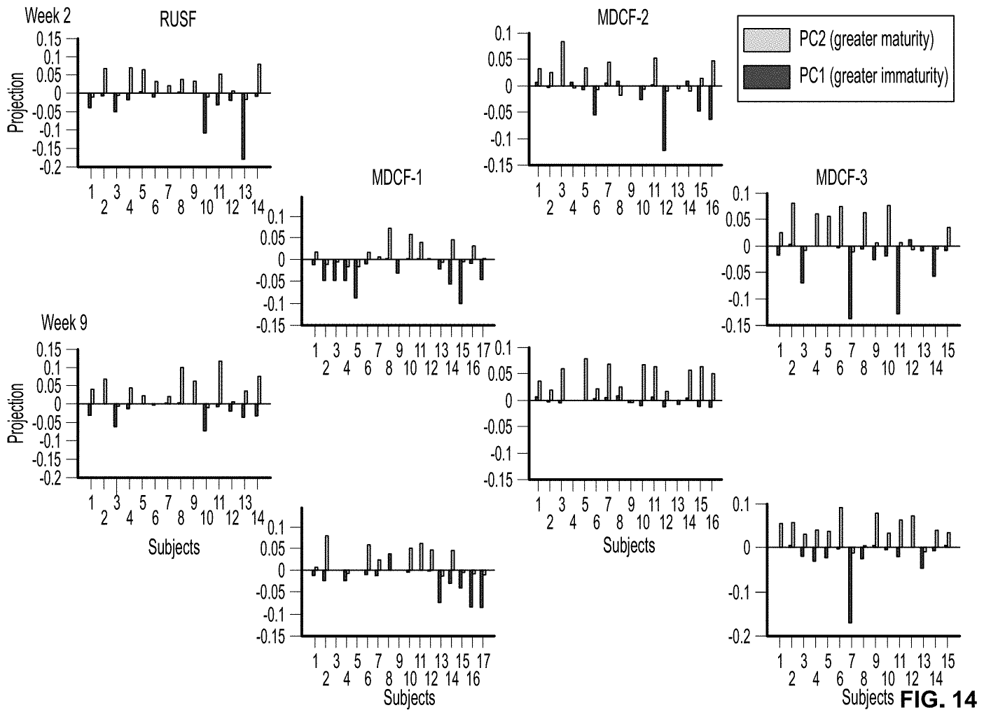

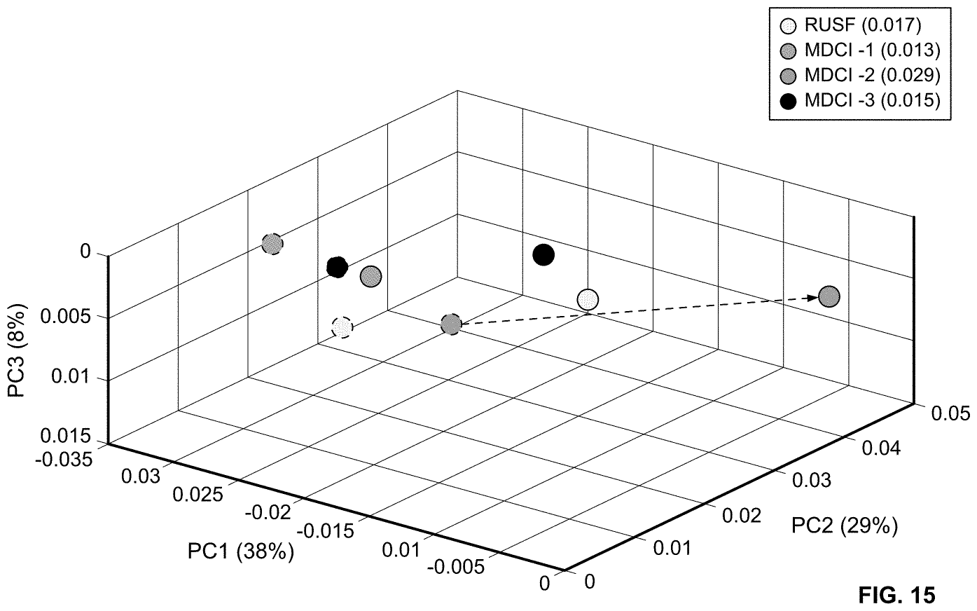

FIGS. 13, 14, and 15 illustrate the effects of microbiota-directed complementary foods (MDCFs) on MAM microbiota configuration. As illustrated in FIG. 13, MAM ecogroup configurations are represented in subjects just prior to treatment (week 2) and 2 weeks after receiving one of the three MDCFs or a control ready-to-use supplementary food (RUSF) (week 9). The bar graphs of FIG. 14, summarizing projections of the individual subjects of FIG. 13 onto PC1 and PC2 demonstrated that MDCF-2 has the greatest effect on microbiota maturity (increased projection on PC2, decreased projection on PC1). FIG. 15 summarizes the centroid of points for each treatment arm calculated at week 2 (dashed black outline) and at week 9 (solid black outline). The distance between the centroids for weeks 2 and 9 for each treatment arm is shown in parenthesis.

FIGS. 16A and 16B illustrate the network configurations of MAM subjects after treatment. The 20-25 month ecogroup network in untreated healthy subjects illustrated in FIG. 16A served as a reference for a graphical representation of the effects of MDCF or RUSF treatments. As illustrated in FIG. 16B, unlike MDCF-1, MDCF-3, and RUSF, MDCF-2 produced an ecogroup configuration that lacked covariance through B. longum; this feature was indicative of a more mature microbiota form.

### Example 5

### Ecogroup Configuration in Severe Acute Malnutrition

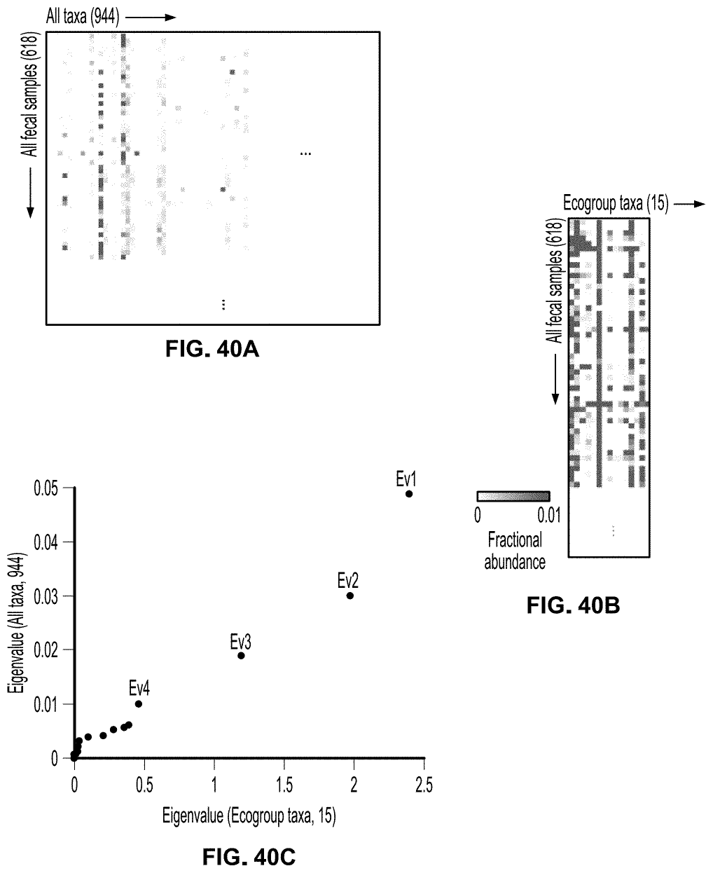

Gehrig et al. describes a clinical trial involving 54 6-36-month-old Bangladeshi children with SAM who were treated with one of three standard therapeutic foods (chickpea, rice-lentil, and ‘plumpy-nut’). A comparison of the eigenspectra computed using all 944 OTUs identified as having a fractional relative abundance of >0.001 in at least 2 of the 618 fecal samples collected prior to, during and after treatment, versus the 15 ecogroup taxa shows that the latter accurately captures sample variance (FIG. 40).

FIGS. 40A, 40B, and 40C illustrate that the ecogroup taxa capture fecal microbiota variance in members of the SAM cohort. FIGS. 40A and 40B are matrices of fractional abundances, constructed from the full microbiota (944 taxa, columns, FIG. 40A) and ecogroup taxa (15 taxa, FIG. 40B), are shown for all fecal samples (n=618) collected during the course of the SAM trial. Eigenspectra (spectrum of eigenvectors (Ev)) are computed over the rows of each matrix. The resulting eigenspectra are plotted against each other in FIG. 40C as an indication of how much sample variance is captured by the ecogroup taxa (x-axis) compared to all taxa (y-axis). Eigenvectors 1-4 are labeled to indicate the reproducibility of detecting primary modes of sample variance when taking into account only ecogroup taxa.

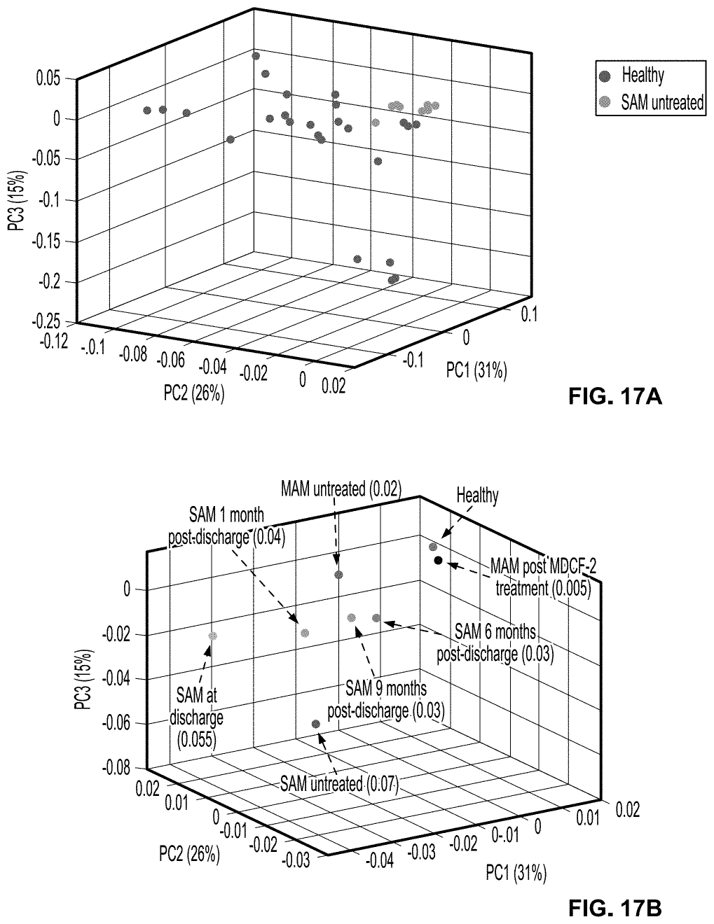

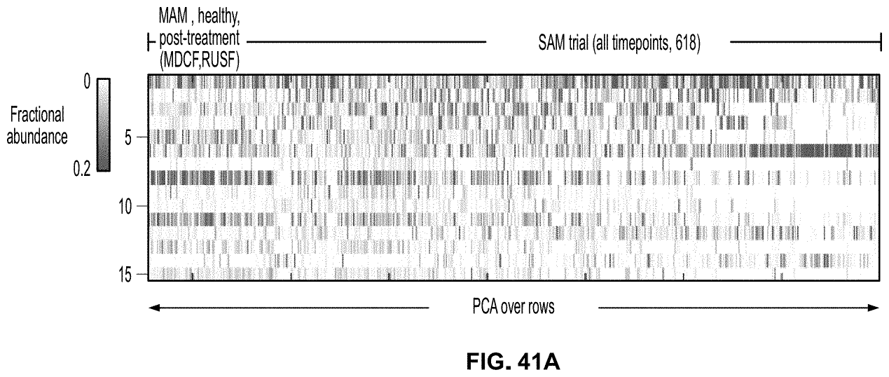

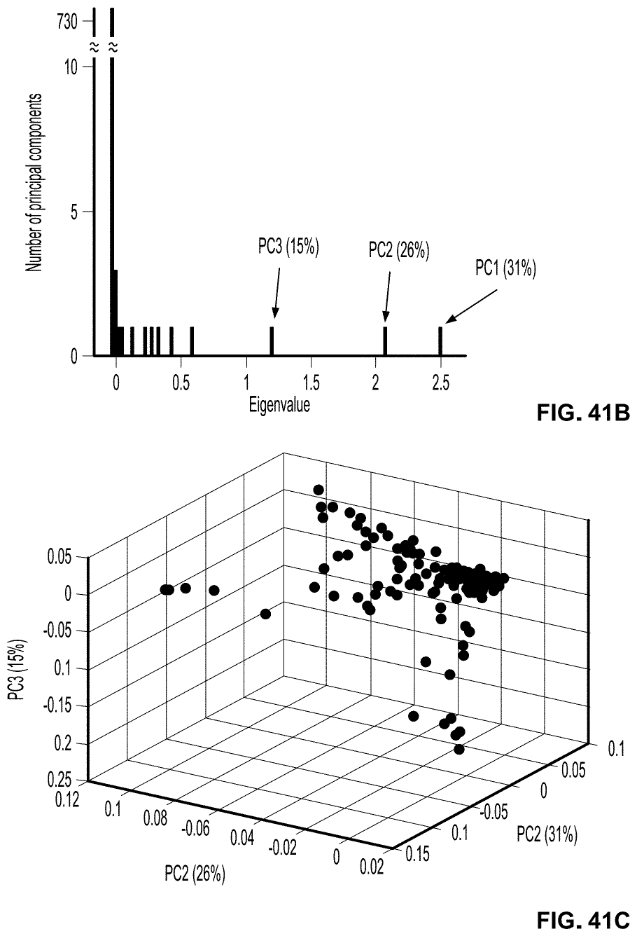

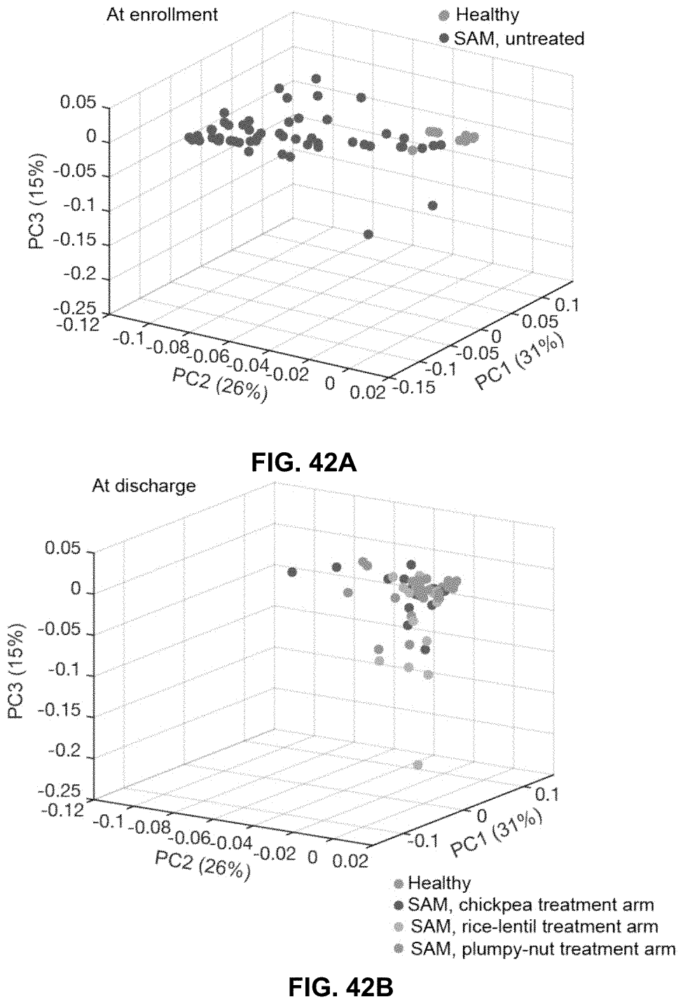

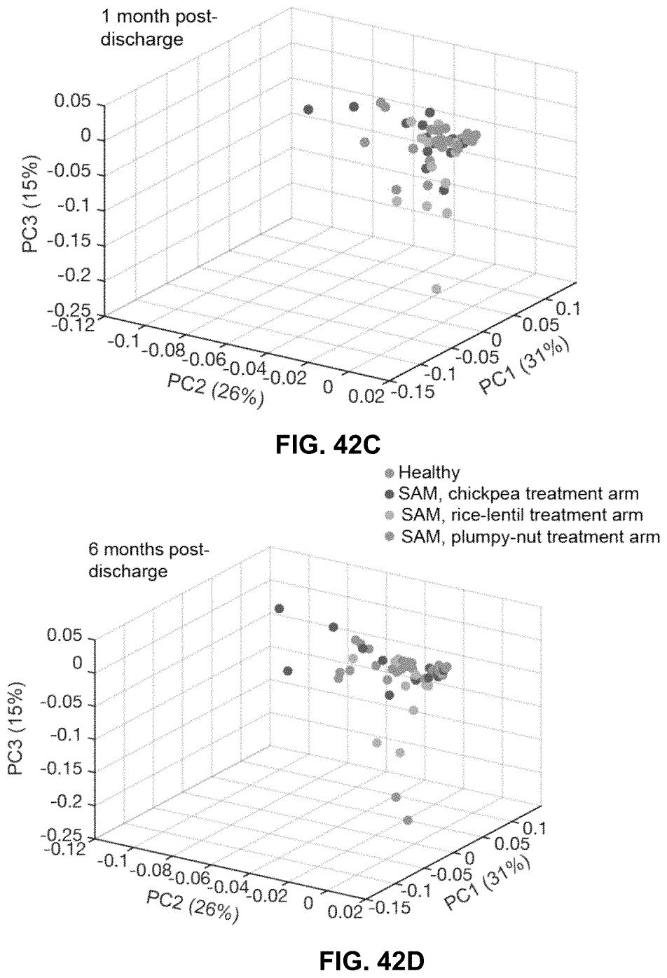

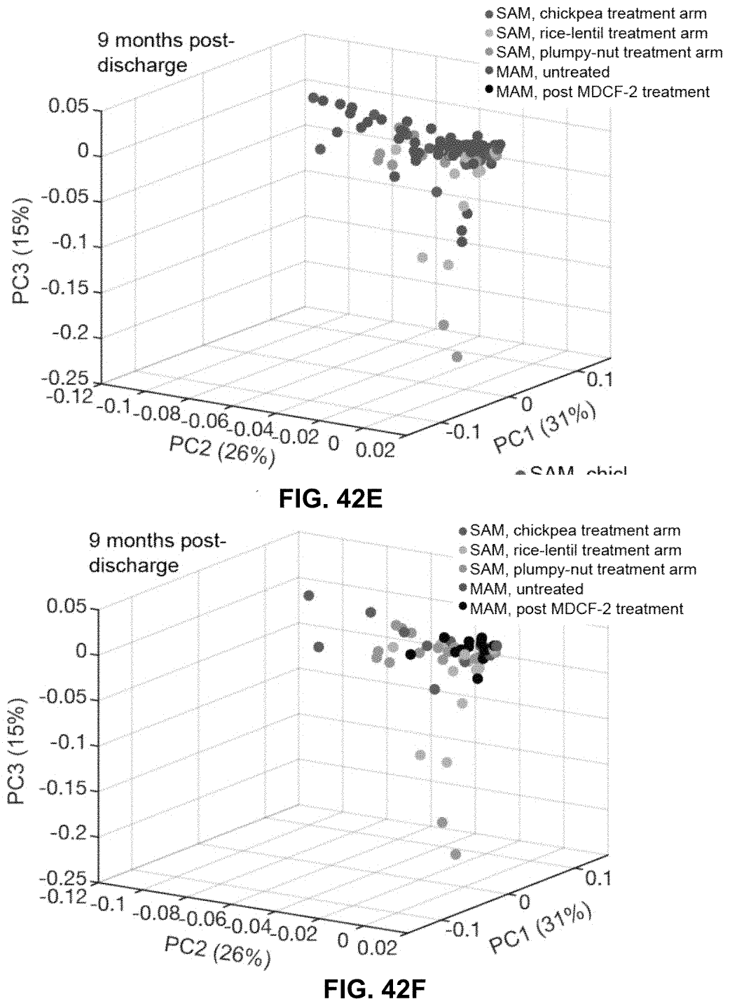

We next created a matrix that included (i) all 618 fecal samples from the SAM trial, (ii) 61 pretreatment samples from children with MAM enrolled in all four arms of the MDCF trial, (iii) 58 MAM samples obtained 2 weeks following treatment with one of the three MDCFs or RUSF, and (iv) 10 fecal samples from 10 age-matched healthy children (FIG. 41A). Each row of the matrix is a fecal sample, each column is an ecogroup taxon, and each element is the fractional abundance of an ecogroup taxon within a particular sample. PCA was performed on the rows of this matrix to generate a space over which comparisons between all cohorts could be made. This computation revealed that three significant principal components define a space onto which each fecal sample could be plotted (FIG. 41B and 41C). The ecogroup-based analysis disclosed that a healthy child's microbiota is localized to the vertex of this space, while gut communities from untreated SAM form a distribution that spreads over all principal components (FIG. 17A and FIG. 42A). At the time of discharge, after receiving standard therapeutic foods, the microbiota of children with SAM remained in an immature state (FIG. 17B). Although there is some improvement 1-month post-discharge, further reconfiguration toward a healthy microbiota is not evident at 6 or 9 months, at which time their microbiota resembled that of untreated children with MAM (FIG. 17B and FIGS. 42B-D). In contrast, the microbiota in children with MAM treated with MDCF-2 overlap nearly perfectly with those of the age-matched healthy children (FIG. 17B, 42E, and 42F).

FIGS. 17A and 17B summarize the ecogroup-based comparison of the microbiota of children with SAM before and after treatment with standard therapy, those with MAM before and after treatment with MDCF-2, and healthy controls. Each axis was constructed as defined in FIG. 41. FIG. 17A shows a comparison of untreated members of the SAM cohort versus healthy controls (red and green dots, respectively). The distance between points was determined by the fractional abundance profile of ecogroup taxa in the microbiota of each child. The closer two dots (children) were in this space, the more similar was their ecogroup taxa profile. Healthy children were centered at the vertex of this space while children with untreated SAM formed a distinct distribution spanning all three principal components. FIG. 17B shows a comparison of the cohort of healthy children against (i) the cohort of children with SAM prior to treatment, at discharge following standard therapy, 1 month post-discharge, 6 months post-discharge, and 9 months post discharge, and (ii) the cohort of children with MAM before treatment and the subcohort who had received MDCF-2. Each dot represents the centroid of the indicated cohort computed within this space. The distance in PCA space of each group of undernourished children from the healthy cohort is indicated in parenthesis.

FIGS. 41A, 41B, and 41C illustrate a process of constructing a PCA space to characterize the ecogroup in the microbiota of SAM cohort members prior to, during and after treatment, MAM cohort members before and after treatment, and in reference healthy controls. FIG. 41A shows a matrix where rows are fecal samples and columns are ecogroup taxa; each element in the matrix is the fractional abundance of an ecogroup taxon within a particular fecal sample. PCA is conducted over rows to compute the variance over fecal samples. FIG. 41B and FIG. 41C demonstrate that PC1, PC2, and PC3 contain the majority of variance in the fecal samples shown in FIG. 41A. These three principal components were used to display the results presented in FIG. 17 and FIG. 42.

FIGS. 42A-42F illustrate a process of using the ecogroup to assess the effects of three standard therapeutic foods on the microbiota of SAM cohort members versus MDCF-2 in children with MAM. Axes for all plots shown were generated as described in FIG. 41. In each plot, a dot represents a single child's ecogroup and the distance between any two points denotes the extent of similarity in the representation of taxa in two individuals' ecogroup. As illustrated in FIG. 42A, at enrollment the fecal microbiota of SAM cohort members (red) have a distinct distribution compared to the microbiota of age-matched healthy controls (green). FIGS. 42B, 42C, and 42D are plots characterizing the microbiota of SAM cohort members as a function of treatment type and timing of sample collection. FIGS. 42E and 42F are PCA plots of all members of the SAM cohort sampled 9 months following receipt of the indicated standard therapy. Ecogroup characterization in all children with primary MAM enrolled in the MDCF trial sampled prior to treatment (FIG. 42E, blue dots) or in the subcohort sampled 2 weeks after MDCF-2 treatment (FIGS. 42F, black dots).

### Example 6

### Human Studies

A previously completed ‘NIH Birth Cohort Study’ (Field Studies of Amebiasis in Bangladesh; ClinicalTrials.gov identifier; NCT02734264) was conducted at the International Centre for Diarrhoeal Disease Research, Bangladesh (icddr,b). Anthropometric data and fecal samples were collected monthly from enrollment through postnatal month 60. Informed consent was obtained from the mother or guardian of each child. The research protocol was approved by the institutional review boards of the icddr,b and the University of Virginia, Charlottesville.

In the case of the MAL-ED birth cohort study (‘Interactions of Enteric Infections and Malnutrition and the Consequences for Child Health and Development’; ClinicalTrials.gov identifier NCT02441426), anthropometric data and fecal samples were collected every month from enrollment to 24 months of age. The study protocol was approved by institutional review boards at each of the study sites.

The accompanying paper by Gehrig et al. describes studies that enrolled (i) Bangladeshi children with MAM in a double-blind, randomized, four group, parallel assignment interventional trial study of microbiota-directed complementary food (MDCF) prototypes (ClinicalTrials.gov identifier NCT03084731) conducted in Dhaka, Bangladesh, (ii) a reference cohort of age-matched healthy children from the same community, and (iii) a subcohort of 54 children with SAM who were treated with one of three different therapeutic foods and followed for 12 months after discharge with serial anthropometry and biospecimen collection ['Development and Field Testing of Ready-to-Use-Therapeutic Foods Made of Local Ingredients in Bangladesh for the Treatment of Children with SAM' (ClinicalTrials.gov Identifier; NCT01889329)] The research protocols for these studies were approved by the Ethical Review Committee at the icddr,b. Informed consent was obtained from the mother/guardian of each child. Use of biospecimens and metadata from each of the human studies for the analyses described in this report was approved by the Washington University Human Research Protection Office (HRPO).

### Example 7

### Collection and Storage of Fecal Samples and Clinical Metadata

Fecal samples were placed in a cold box with ice packs within 1 hour of production by the donor and collected by field workers for transport back to the lab (NIH Birth Cohort, MAL-ED study). For the ‘Development and Field Testing of Ready-to-Use-Therapeutic Foods Made of Local Ingredients in Bangladesh for the Treatment of Children with SAM’ study, the healthy reference cohort, and the MDCF trial, samples were flash frozen in liquid nitrogen-charged dry shippers (CX-100, Taylor-Wharton Cryogenics) shortly after their production by the infant or child. Biospecimens were subsequently transported to the local laboratory and transferred to −80° C. freezers within 8 hours of collection. Samples were shipped on dry ice to Washington University and archived in a biospecimen repository at −80° C.

### Example 8

### Sequencing Bacterial V4-16S rRNA Amplicons and Assigning Taxonomy

Methods used for isolation of DNA from frozen fecal samples, generation of V4-16S rDNA amplicons, sequencing of these amplicons, clustering of sequencing reads into 97% ID OTUs and assigning taxonomy are described in Gehrig et al.

### Example 9

### Generation of RF-Derived Models of Gut Microbiota Development

We produced RF-derived models of gut microbiota development from the Peruvian, Indian, and ‘aggregate’ V4-16S rRNA datasets generated from 22, 14, and 28 healthy participants, respectively. Model building for each birth cohort was initiated by regressing the relative abundance values of all identified 97% ID OTUs in all fecal samples against the chronologic age of each donor at the time each sample was procured (R package ‘randomForest’, ntree=10,000). For each country site, OTUs were ranked based on their feature importance scores, calculated from the observed increases in mean square error (MSE) rate of the regression when values for that OTU were randomized. Feature importance scores were determined over 100 iterations of the algorithm. To determine how many OTUs were required to create a RF-based model comparable in accuracy to a model comprised of all OTUs, we performed an internal 100-fold cross-validation where models with sequentially fewer input OTUs were compared to one another. Limiting the country-specific models to the top 30 ranked OTUs had only minimal impact on accuracy (within 1% of the mean squared error obtained with all OTUs). In addition to calculating the R² of the chronological age vs. predicted microbiota age for reciprocal cross-validation of the RF-derived models, we also calculated the mean absolute error (MAE) and root mean squared error (RMSE) for the application of each model to each dataset to further assess model quality.

### Example 10

### Generating Ecogroup Network Graphs

Nodes in all graphs are 97% ID OTUs. An edge connected taxa i and j if the absolute value of their normalized covariance was within the top 20% of the probability distribution of all values. Applying this threshold creates an ‘adjacency’ matrix that serves as direct input for generating an undirected network graph. The adjacency matrix was defined as a matrix of ones and zeros where a matrix element entry of ‘1’ is indicative of a connection between the corresponding row and column. Network graphs were constructed using the open-source software package Gephi. Nodes in FIG. 7 were sized according to the ‘degree’ (number) of edges present at that node and edges were weighted according to the (C_{bin}^{i,j})ₜ value between species i and j. Nodes in FIGS. 10, 11, 12, 13, and 16 were sized according to average fractional abundances of taxa with equal edge weights. The force-atlas 2 force-field was applied to spatially separate networks.

### Example 11

### Comparing OTUs with DADA2 Amplicon Sequence Variants (ASVs)

Each OTU in the ecogroup and each OTU in the sparse RF-derived models that had 100% sequence identity to an ASV was identified; each of these OTUs was defined as a ‘primary OTU sequence’ and the ASV as the ‘correct ASV sequence’. The primary OTU sequence was then mutated according to the maximum sequence variance accepted by QIIME for a 97% ID OTU (i.e. 3%) to create a library of 1000 derivative sequences. Each sequence in the library was then compared to a database of all ASVs produced from DADA2 analysis of all 16S rDNA datasets generated from all birth cohorts described in this report and. The ASV with the maximum sequence identity to each member of each library of 1000 derivative sequences was noted. If this ASV matched the ‘correct ASV sequence’ the OTU derivative sequence in the library was assigned a ‘1’, otherwise it was ascribed a ‘0’. An average over all 1000 derivative sequences in a given library was then calculated. This process was iterated 10 separate times, creating 10 trials of 1000 derived sequences for each OTU. An overall average over all 10 trials was then calculated, thereby defining the probability of an OTU being ascribed to the correct ASV given the accepted sequence ‘entropy’ of QIIME. The results demonstrated that V4-16S rDNA sequences comprising a 97%ID OUT generated by QIIME map directly to the single ASV sequence deduced by DADA2.

FIGS. 18A, 18B, 18C, and 18D illustrate a comparison of taxonomic assignments generated by QIIME and Amplicon Sequence Variants (ASVs) by DADA2. Sensitivity analysis directly compared taxonomic assignments using OTUs versus amplicon sequence variants (ASVs). FIG. 18A shows a summary of the workflow. An ASV database is created by running all datasets described in this report and through the DADA2 pipeline. 15 ASVs are randomly chosen; for each ASV, a V4-16S rDNA sequence that has 100% identity with the ASV is defined as the ‘primary OTU sequence’. For each ‘primary OTU sequence’, a library of 1000 sequences with at least 97% identity is generated. Each sequence in each library is then compared to the ASV database and the ASV with the maximum sequence identity (MSI) is noted. If the MSI ASV corresponds to the ASV from which the ‘primary OTU sequence’ was generated (defined as the ‘correct ASV’), the sequence in the library is given a ‘1’. Otherwise, the sequence in the library is given a ‘0’. An average score is computed for the entire library. The process of library generation and sequence score designation is repeated 10 times and an overall average is computed. This average represents the probability of assigning the ‘correct ASV’ to the ‘primary OTU sequence’ given a sequence divergence of 3%. 10 separate iterations of the procedure described in FIG. 18A were conducted, each generating 15 randomly chosen sequences from the ASV database. Corresponding ‘primary OTU sequences’ were identified from all birth cohorts studied. The probability of detecting the ‘correct ASV’ for each of the 15 randomly chosen ‘primary OTU sequences’ is shown in the bar plot with errors corresponding to the standard deviation of the probability. The procedure described in FIG. 18A was applied to the 15 ecogroup taxa (FIG. 18C) and the 30 taxa comprising the 2-year sparse Bangladeshi RF-generated model (FIG. 18D).

### Example 12

### The Effect of Bacterial Load on Ecogroup Definition

Given a set of N total fecal samples where each fecal sample (microbiota) contains a set of taxa, the fractional representation of any taxon can be calculated as

bᵢxᵢ=Xᵢ (1)

where bᵢ and xᵢ and Xᵢ represent the ‘bacterial load’, fractional abundance, and total abundance, respectively, of taxon ‘x’ for microbiota i. The covariance between taxon ‘x’ and taxon ‘y’ can be represented as

$$cov ( x , y ) = \frac{1}{N} {\sum }_{i = 1}^{N} ( {x}_{i} - 〈 x 〉 ) ( {y}_{i} - 〈 y 〉 ) \qquad ( 2 )$$

The average fractional abundance of a taxon ‘x’, for instance, can be expressed in terms of bacterial load as the following

$$〈 x 〉 = \frac{1}{N} {\sum }_{i = 1}^{N} \frac{{X}_{i}}{{b}_{i}} \qquad ( 3 )$$

Substituting Eqn. (3) into Eqn. (2) gives

$$cov ( x , y ) = \frac{1}{N} {\sum }_{i = 1}^{N} [ {x}_{i} - \frac{1}{N} ( \frac{{X}_{1}}{{b}_{1}} + \frac{{X}_{2}}{{b}_{2}} + ⋯ + \frac{{X}_{N}}{{b}_{N}} ) ] [ {y}_{i} - \frac{1}{N} ( \frac{{Y}_{1}}{{b}_{1}} + \frac{{Y}_{2}}{{b}_{2}} + ⋯ + \frac{{Y}_{N}}{{b}_{N}} ) ] \qquad ( 4 )$$

The fractional abundance of taxon ‘x’ for any microbiota i can be expressed as total abundance and fractional abundance from Eqn. (1) as

$${x}_{i} = \frac{{X}_{i}}{{b}_{i}} \qquad ( 5 )$$

Substituting this into Eqn. (4) gives

$$cov ( x , y ) = \frac{1}{N} {\sum }_{i = 1}^{N} [ \frac{{X}_{i}}{{b}_{i}} - \frac{1}{N} ( \frac{{X}_{1}}{{b}_{1}} + \frac{{X}_{2}}{{b}_{2}} + ⋯ + \frac{{X}_{N}}{{b}_{N}} ) ] [ \frac{{Y}_{i}}{{b}_{i}} - \frac{1}{N} ( \frac{{Y}_{1}}{{b}_{1}} + \frac{{Y}_{2}}{{b}_{2}} + ⋯ + \frac{{Y}_{N}}{{b}_{N}} ) ] \qquad ( 6 )$$

Given the expression shown in Eqn. (5), we can now address the case where (1) bacterial load is constant across all fecal samples, and (2) bacterial load is different across fecal samples.

Case 1: All Bacterial Loads Are Equal Across All N Microbiota

In the case that bacterial loads are equal across all N fecal samples,

b₁=b₂= . . . =b_{N} (7)

Thus, bᵢ can be substituted for b, a constant bacterial load across all N. Substituting this into Eqn. (5) gives

$$cov ( x , y ) = \frac{1}{N} {\sum }_{i = 1}^{N} [ \frac{{X}_{i}}{b} - \frac{1}{N} ( \frac{{X}_{1}}{b} + \frac{{X}_{2}}{b} + ⋯ + \frac{{X}_{N}}{b} ) ] [ \frac{{Y}_{i}}{b} - \frac{1}{N} ( \frac{{Y}_{1}}{b} + \frac{{Y}_{2}}{b} + ⋯ + \frac{{Y}_{N}}{b} ) ] \qquad ( 8 )$$

Eqn. (6) simplifies to

$$cov ( x , y ) = \frac{1}{bN} {\sum }_{i = 1}^{N} [ {X}_{i} - \frac{1}{N} ( {X}_{1} + {X}_{2} + ⋯ + {X}_{N} ) ] [ {Y}_{i} - \frac{1}{N} ( {Y}_{1} + {Y}_{2} + ⋯ + \vert {Y}_{N} ) ] \qquad ( 9 )$$

which is equal to

$$cov ( x , y ) = \frac{1}{bN} {\sum }_{i = 1}^{N} [ {X}_{i} - 〈 X 〉 ] [ {Y}_{i} - 〈 Y 〉 ] \qquad ( 10 )$$

Covariance calculated using absolute bacterial load between two taxa, ‘X’ and ‘Y’ is

$$cov ( X , Y ) = \frac{1}{N} {\sum }_{i = 1}^{N} [ {X}_{i} - 〈 X 〉 ] [ {Y}_{i} - 〈 Y 〉 ] \qquad ( 11 )$$

Thus from Eqn. (10) and Eqn. (11)

$$cov ( x , y ) = \frac{1}{b} cov ( X , Y ) \qquad ( 12 )$$

The result of Eqn. (12) illustrates that when taking into account a constant bacterial load across an ensemble of fecal samples, the covariance computed between taxa ‘x’ and ‘y’ and between ‘X’ and ‘Y’ are related to each other by a constant—the inverse of the bacterial load.

In our statistical approach, temporally conserved taxon-taxon covariance is computed using fractional abundance measurements from month 21 to 60 of postnatal life across the healthy Mirpur cohort. If we were to take into account a constant bacterial load across all samples, this covariance matrix would scale in a directly proportionate fashion.

The next step in our approach is to apply PCA to the temporally weighted covariance matrix. The first step of PCA is to compute the eigenvectors and eigenvalues of the input matrix. We can ask what is the effect of proportionately scaling data with respect to identifying eigenvalues and eigenvectors of a matrix? Given the temporally weighted covariance matrix C, the way to identify the eigenvalues of C is by solving

det(C−ΩI)=0 (13)

where ‘det’ means determinant, I is the identity matrix of the same dimension as C and Ω represents the eigenvalues to be solved. As an example, if C is a 2×2 matrix defined as

$$C = [ \begin{matrix}{C}_{11} & {C}_{12} \\ {C}_{21} & {C}_{22}\end{matrix} ] \qquad ( 14 )$$

then substituting Eqn. (14) into Eqn. (13) becomes

$$det ( [ \begin{matrix}{C}_{11} & {C}_{12} \\ {C}_{21} & {C}_{22}\end{matrix} ] - Ω [ \begin{matrix}1 & 0 \\ 0 & 1\end{matrix} ] ) = 0 \qquad ( 15 )$$

which equals

$$det ( [ \begin{matrix}{C}_{11} & {C}_{12} \\ {C}_{21} & {C}_{22}\end{matrix} ] - [ \begin{matrix}Ω & 0 \\ 0 & Ω\end{matrix} ] ) = 0 \qquad ( 16 )$$

which equals

$$det ( [ \begin{matrix}{C}_{11} - Ω & {C}_{12} \\ {C}_{21} & {C}_{22} - Ω\end{matrix} ] ) = 0 \qquad ( 17 )$$

Computing Eqn. (17) yields

(C₁₁−Ω)(C₂₂−Ω)−C₁₂C₂₁=0 (18)

To compute the eigenvalues of the matrix C, solve Eqn. (18) for 12. Expanding Eqn. (18) yields

C₂₂C₁₁−ΩC₁₁−ΩC₂₂+Ω²−C₁₂C₂₁=0 (19)

The trace of C (Tr(C), sum of elements on main diagonal of C) is

C₁₁+C₂₂=Tr(C) (20)

The determinant of C is defined as

C₂₂C₁₁−C₁₂C₂₁=det(C) (21)

Therefore Eqn. (19) can be expressed as

Ω²−ΩTr(C)+det(C)=0 (22)

Using the quadratic formula to solve for 12 in Eqn. (22) gives the following solution for the eigenvalues of C

$$\frac{1}{2} [ Tr ( C ) \pm \sqrt[2]{{( Tr ( C ) )}^{2} - 4 ( det ( C )} ] = Ω \qquad ( 23 )$$

If the matrix C is scaled by a proportion b, as would be the case for an equal bacterial load across all samples, Eqn. (16) becomes

$$det ( [ \begin{matrix}{bC}_{11} & {bC}_{12} \\ {bC}_{21} & {bC}_{22}\end{matrix} ] - [ \begin{matrix}Ω & 0 \\ 0 & Ω\end{matrix} ] ) = 0 \qquad ( 24 )$$

which equals

$$det ( [ \begin{matrix}{bC}_{11} - Ω & {bC}_{12} \\ {bC}_{21} & {bC}_{22} - Ω\end{matrix} ] ) = 0 \qquad ( 25 )$$

Computing Eqn. (25) yields

(bC₁₁−Ω)(bC₂₂−Ω)−b²C₁₂C₂₁=0 (26)

Expanding Eqn. (26) yields

b²C₂₂C₁₁−ΩbC₁₁ΩbC₂₂Ω²−b²C₁₂C₂₁=0 (27)

Using the definition of the trace and determinant of matrix C from Eqns. (20), (21), and (27) can be expressed as

Ω²−bΩTr(C)+b²det(C)=0 (28)

Using the quadratic formula to solve for 12 in Eqn. (28) gives the following solution for the eigenvalues of C scaled by b.

$$\frac{1}{2} [ bTr ( C ) \pm \sqrt[2]{{{b}^{2} ( Tr ( C ) )}^{2} - 4 {b}^{2} ( det ( C )} ] = Ω \qquad ( 29 )$$

Eqn. (29) can be simplified to

$$\frac{1}{2} b [ Tr ( C ) \pm \sqrt[2]{{( Tr ( C ) )}^{2} - 4 ( det ( C )} ] = Ω \qquad ( 30 )$$

Using Eqn. (23) as the solution for the unscaled eigenvalues, Ω_{unscaled}, and Eqn. (29) as the solution for the scaled eigenvalues, Ω_{scaled}, Eqn. (23) and Eqn. (29) can be related to each other by the following

b[Ω_{unscaled}]=Ω_{scaled} (31)

Thus, taking into account a constant bacterial load across all samples scales the eigenvalues for each eigenvector by the constant bacterial load b. If a matrix is scaled by a proportion, we can ask whether this affects the eigenvectors (principal components). The fundamental relationship between a square matrix C, eigenvector v, and eigenvalue Ω is

Cv=Ωv (32)

If C is scaled by a constant b,

(bC)v=b(Cv)=b(Ωv) (33)

Thus, scaling the matrix C does not affect the eigenvectors of the matrix, but only affects their scaling, i.e., eigenvalues. An example of this result is shown in FIG. 26A.

FIG. 26A illustrates the effect of bacterial load on identifying groups of co-varying taxa by PCA. FIG. 26A shows a sample 4 by 4 unscaled matrix is shown with values ranging from −1 to 1 (upper portion). The eigenspectrum of this matrix is displayed showing 4 eigenvectors (Ev) with corresponding eigenvalues. The columns of this matrix are scaled to represent a constant bacterial load across all fecal samples (lower portion). The eigenspectrum of the scaled matrix is displayed. The eigenvalues of the scaled and unscaled eigenvectors are plotted against each other, illustrating a linear relationship.

Case 2: All Bacterial Loads Differ Across All N Microbiota

If bacterial loads are different between samples, the simplification from (6) to (8) no longer holds. Thus, as a simple example of how different bacterial loads affect covariance between taxa, assume N=2. Therefore,

$$cov ( x , y ) = \frac{1}{2} [ ( \frac{{X}_{1}}{{b}_{1}} - 〈 x 〉 ) ( \frac{{X}_{2}}{{b}_{2}} - 〈 x 〉 ) ] [ ( \frac{{Y}_{1}}{{b}_{1}} - 〈 y 〉 ) ( \frac{{Y}_{2}}{{b}_{2}} - 〈 y 〉 ) ] \qquad ( 34 )$$

Eqn. (34) can be expanded to

$$cov ( x , y ) = \frac{1}{2} [ \frac{{X}_{1}}{{b}_{1}} \frac{{X}_{2}}{{b}_{2}} - \frac{{X}_{1}}{{b}_{1}} 〈 x 〉 - \frac{{X}_{2}}{{b}_{2}} 〈 x 〉 - {〈 x 〉}^{2} ] [ \frac{{Y}_{1}}{{b}_{1}} \frac{{Y}_{2}}{{b}_{2}} - \frac{{Y}_{1}}{{b}_{1}} 〈 y 〉 - \frac{{Y}_{2}}{{b}_{2}} 〈 y 〉 - {〈 y 〉}^{2} ] \qquad ( 35 )$$

Expanding Eqn. (35) gives

$$cov ( x , y ) = \frac{1}{2 {b}_{1} {b}_{2}} [ {X}_{1} {X}_{2} {Y}_{1} {Y}_{2} - {X}_{1} {X}_{2} 〈 y 〉 ( {b}_{2} {Y}_{1} + {b}_{1} {Y}_{2} ) - {X}_{1} {X}_{2} {b}_{1} {b}_{2} {〈 y 〉}^{2} + 〈 x 〉 〈 y 〉 [ {b}_{2} {X}_{1} + {b}_{1} {X}_{2} ] [ {b}_{2} {Y}_{1} + {b}_{1} {Y}_{2} ] - 〈 x 〉 ( {b}_{2} {X}_{1} + {b}_{1} {X}_{2} ) {Y}_{1} {Y}_{2} + 〈 x 〉 ( {b}_{2} {X}_{1} + {b}_{1} {X}_{2} ) {b}_{1} {b}_{2} {〈 y 〉}^{2} - {b}_{1} {b}_{2} {〈 x 〉}^{2} {Y}_{1} {Y}_{2} + {〈 x 〉}^{2} {b}_{1} {b}_{2} 〈 y 〉 ( {b}_{2} {Y}_{1} + \vert {b}_{1} {Y}_{2} ) + {b}_{1}^{2} {b}_{2}^{2} {〈 x 〉}^{2} {〈 y 〉}^{2} ] \qquad ( 36 )$$

If only fractional abundance is taken into consideration, the covariance between fractional abundance of taxa ‘x’ and ‘y’ over N=2 is

$$cov ( x , y ) = \frac{1}{2} [ {x}_{1} {x}_{2} {y}_{1} {y}_{2} - {x}_{1} {x}_{2} 〈 x 〉 ( {y}_{1} + {y}_{2} ) - {x}_{1} {x}_{2} {〈 y 〉}^{2} + 〈 x 〉 〈 y 〉 [ {x}_{1} + {x}_{2} ] [ {y}_{1} + {y}_{2} ] - 〈 x 〉 ( {x}_{1} + {x}_{2} ) {y}_{1} {y}_{2} + 〈 x 〉 ( {x}_{1} + {x}_{2} ) {〈 y 〉}^{2} - {〈 x 〉}^{2} {y}_{1} {y}_{2} + {〈 x 〉}^{2} 〈 y 〉 ( {y}_{1} + {y}_{2} ) + {〈 x 〉}^{2} {〈 y 〉}^{2} ] \qquad ( 37 )$$

Comparing Eqn. (36) with Eqn. (37) shows that taking into consideration differential bacterial load across the two samples scales each term in the equations by a combination of the bacterial loads for each sample in a non-linear fashion. Thus, unlike the case where a constant bacterial load across fecal samples scales the eigenvalues for each eigenvector by the bacterial load, in this case, the relationship is a non-linear scaling, with the exact value of scaling being dependent on the value of each bacterial load. As illustrated in the accompanying figure using a toy example of differential bacterial load across samples, FIG. 26B, we see that though the eigenvalues for each eigenvector scale non-linearly, the eigenvectors themselves remain unchanged. Mathematically this is consistent with Eqn. (32). If bacterial load is taken into consideration, the input values to the covariance calculation will be absolutely different, as shown by Eqn. (1), but a relative measure of whether two taxa co-vary does not change. Mathematically, by Eqn. (32), the set of eigenvalues for each eigenvector might be different, but the existence of the set of eigenvectors v (a set of transformed axes to represent the data) remains unchanged. The practical interpretation of this result is that while taking into consideration differential bacterial load will change the significance of the principal component (its eigenvalue, and therefore the amount of variance carried by the principal component), PC1 remains PC1 even if a differential bacterial load across samples is considered. Thus, identification of ecogroup taxa (taxa that project significantly onto PC1 of a temporally conserved covariance matrix as defined in FIG. 5A), is invariant to differential bacterial load.

FIG. 26B further illustrates the effect of bacterial load on identifying groups of co-varying taxa by PCA. Differential scaling of the unscaled matrix in is performed to represent different bacterial loads across fecal samples. The unscaled and differentially scaled matrices and eigenspectra are shown. The eigenvalues of the unscaled and differentially scaled eigenvectors are plotted against each other, illustrating a near-linear relationship.

### Example 13

### Dietary Practices and Ecogroup Development

The daily diets of members of the 5-year Mirpur birth cohort were recorded from postnatal day 1 through to 60 months. Diet profiles of each of the 37 individuals are shown in FIGS. 40A-C where a dietary transition is defined if a new diet category was consumed for >30 days. The time at which ecogroup configuration 1 achieves maturity (month 23) corresponded to the time that weaning from breast milk was completed (FIG. 31). In contrast, the time at which ecogroup configurations 2, 3, 4, and 5 achieved maturity (months 9 and 26) did not qualitatively correlate with a fully weaned state. Interestingly, although ecogroup configurations 1 and 5 mature at approximately the same time (23 and 26 months, respectively), children having microbiota with ecogroup configuration 5 wean off breast milk later on average than those with ecogroup configuration 1 (37 vs 22 months; FIG. 31). A caveat is that we likely currently lack sufficient numbers of individuals, as well as sufficient granularity regarding complementary feeding practices to relate specific changes in diet to reproducible changes in ecogroup structure.

### Example 14

### Generating RF-Derived Models of Gut Microbiota Development in Healthy Members of Birth Cohorts Representing Geographically Distinct Regions and Anthropologic Characteristics

MAL-ED is a network of eight study sites, located in low-income countries, dedicated to assessing the impact of enteric infections that alter gut function and impair the growth and development of infants and children. To define the extent to which age-discriminatory taxa are shared between infants and young children, we generated V4-16S rRNA datasets from fecal samples collected monthly for the first 2 postnatal years from members of MAL-ED birth cohorts with healthy growth phenotypes living in Loreto, Peru, Vellore, India, Fortaleza, Brazil and Venda, South Africa [n=22.4±2.8 (mean±SD) fecal samples/child; total of 1639 samples]. ‘Healthy’ in these sites was defined as height-for-age and weight-for-height Z-scores (HAZ, WHZ) consistently no more than 1.5 standard deviations below the median calculated from a WHO reference healthy growth cohort. Bacterial V4-16S rDNA reads were grouped into 97% ID OTUs.

Using the 16S rDNA dataset and a sparse 2-year, 30 OTU RF-derived model generated from 25 healthy members of the Bangladeshi birth cohort, we determined that a minimum of 12 individuals would be required to construct a model with comparable performance (FIGS. 34A, 34B, and 34C). Based on this result, we generated RF-derived models of gut development from the sufficiently powered Indian and Peruvian datasets (FIGS. 34D and 34E). Limiting models to 30 OTUs with the top ranked feature importance scores had only minimal impact on accuracy (i.e., the models were within 1% of the mean squared error obtained using all OTUs). Therefore, our subsequent analyses used sparse site-specific RF-generated models that were each comprised of their 30 top-ranked 97% ID OTUs. The Peruvian and Indian models shared 13 OTUs with each other, and 16 and 15 OTUs with the Bangladeshi model, respectively (FIGS. 34D and 34E).

We created a sparse ‘aggregate’ model from bacterial 16S rRNA datasets generated from all but the Bangladeshi birth cohort (i.e., the MAL-ED cohorts from India, Peru, Brazil and South Africa) (FIGS. 35A and 35B). To balance the representation of each site's contribution to the aggregate model, seven of the most densely sampled healthy individuals from each of the four sites were selected (n=599 fecal samples). The resulting RF-derived aggregate model contained 17 of the 30 OTUs present in the sparse 2-year Bangladeshi RF-derived model, and 18 and 16 of the OTUs in the sparse Indian and Peruvian models, respectively (also see FIG. 35C).

What is claimed is:

1. A computer-implemented method for characterizing a gut microbiome of a group of subjects, the method comprising:
providing a microbiome dataset comprising a plurality of entries, each entry comprising a plurality of microbial taxa and associated abundances, each entry further comprising at least one subject classification selected from an age, a health condition, a treatment condition, and a geographical location;
transforming a first portion of the microbiome dataset into a first eigenspectrum;
transforming at least one additional portion of the microbiome dataset into at least one additional eigenspectrum;
comparing corresponding components of the first eigenspectrum and the at least one additional eigenspectrum; and
characterizing the gut microbiome based on the comparison of the first eigenspectrum and the at least one additional eigenspectrum;
wherein each of the first eigenspectrum and the at least one additional eigenspectrum comprises a plurality of eigenvectors and associated eigenvalues.

2. The computer-implemented method of claim 1, wherein the plurality of microbial taxa and associated abundances includes at least one measurement selected from the group consisting of genomic measurements, gene expression measurements, proteomic measurements, and metabolite measurements.

3. The computer-implemented method of claim 1, wherein the abundances of microbial taxa are determined by analysis of fecal samples.

4. The computer-implemented method of claim 3, wherein the fecal samples provide a plurality of reads to a computing device that are analyzed to calculate an alpha diversity and/or a beta diversity of the taxa within the microbiome.

5. The computer-implemented method of claim 3, wherein the fecal samples are taken from a subject or a subject group at least two different times.

6. The computer-implemented method of claim 5, wherein the two different times are selected to capture different ages or developmental stages of the subject or subject group.

7. The computer-implemented method of claim 5, wherein fecal samples are taken before, during, and after administration of a therapeutic intervention.

8. The computer-implemented method of claim 3, wherein the computing device transforms the relative fractional abundances of one or more fecal samples to enable characterizing of at least one aspect of the microbiome.

9. The computer-implemented method of claim 8, wherein the at least one aspect of the microbiome is selected from the group consisting of covariance of taxa, and/or microbiome configurations representative of subject populations including healthy subjects at various developmental stages, subjects with various gastrointestinal conditions such as malnutrition, and subjects at various stages of treatment for a gastrointestinal condition.

10. The computer-implemented method of claim 1, wherein:
characterizing the gut microbiome comprises monitoring an effect of a treatment for a gastrointestinal condition using a treatment comprising a plurality of phases;
the first portion comprises a combination of all entries of the plurality of entries of the microbiome dataset with a health condition of healthy;
each additional portion comprises a combination of all entries of the plurality of entries with a health condition of gastrointestinal condition and a treatment condition classified as undergoing one phase of the plurality of phases of the treatment; and
monitoring the effect of the treatment comprises;
transforming the first eigenvector and each additional eigenvector into a separation distance; and
a reduction in separation distance between an earlier phase and a later phase of a treatment indicates an efficacy of the treatment.

11. The method of claim 1, wherein:
characterizing the gut microbiome comprises identifying a microbiome configuration age to achieve a stable microbiome configuration;
the first portion comprises a combination of all entries of the plurality of entries of the microbiome dataset comprising the subject classifications of the youngest age and the oldest age;
each additional portion comprises a successively larger portion of the plurality of entries, the successively larger portion comprising all entries of the plurality of entries of the microbiome dataset comprising the subject classifications of the youngest age, the oldest age, and successively larger portions of the ages between the youngest age and the oldest age;
comparing corresponding components of the first eigenspectrum and the at least one additional eigenspectrum comprises comparing each first eigenvalue associated with each first eigenvector of each eigenspectrum; and
characterizing the gut microbiome based on the comparison of the first eigenspectrum and the at least one additional eigenspectrum comprises:
identifying the stable eigenspectrum from the at least one additional eigenspectrum at which the first eigenvalue reaches an asymptotic value; and
identifying the age added to generate the additional portion of the entries transformed into the stable eigenspectrum as the age to achieve a stable microbiome configuration.

12. The computer-implemented method of claim 2, wherein there are at least two measurements.

13. A computer-implemented method for monitoring changes in the gut microbiome of a group of subjects, the method comprising:
providing a microbiome dataset comprising a plurality of entries, each entry comprising a plurality of microbial taxa and associated abundances, each entry further comprising at least one subject classification selected from an age, a health condition, a treatment condition, and a geographical location;
transforming a first portion of the microbiome dataset into a first eigenspectrum;
transforming at least one additional portion of the microbiome dataset into at least one additional eigenspectrum;
comparing corresponding components of the first eigenspectrum and the at least one additional eigenspectrum; and
monitoring changes in the gut microbiome based on the comparison of the first eigenspectrum and the at least one additional eigenspectrum;
wherein each of the first eigenspectrum and the at least one additional eigenspectrum comprises a plurality of eigenvectors and associated eigenvalues.

14. The method of claim 13, wherein:
monitoring changes in the gut microbiome comprises identifying a microbiome configuration age to achieve a stable microbiome configuration;
the first portion comprises a combination of all entries of the plurality of entries of the microbiome dataset comprising the subject classifications of the youngest age and the oldest age;
each additional portion comprises a successively larger portion of the plurality of entries, the successively larger portion comprising all entries of the plurality of entries of the microbiome dataset comprising the subject classifications of the youngest age, the oldest age, and successively larger portions of the ages between the youngest age and the oldest age;
comparing corresponding components of the first eigenspectrum and the at least one additional eigenspectrum comprises comparing each first eigenvalue associated with each first eigenvector of each eigenspectrum; and
monitoring changes in the gut microbiome based on the comparison of the first eigenspectrum and the at least one additional eigenspectrum comprises:
identifying the stable eigenspectrum from the at least one additional eigenspectrum at which the first eigenvalue reaches an asymptotic value; and
identifying the age added to generate the additional portion of the entries transformed into the stable eigenspectrum as the age to achieve a stable microbiome configuration.

15. The computer-implemented method of claim 13, wherein:
monitoring changes in the gut microbiome comprises monitoring an effect of a treatment for a gastrointestinal condition using a treatment comprising a plurality of phases;
the first portion comprises a combination of all entries of the plurality of entries of the microbiome dataset with a health condition of healthy;
each additional portion comprises a combination of all entries of the plurality of entries with a health condition of gastrointestinal condition and a treatment condition classified as undergoing one phase of the plurality of phases of the treatment; and
monitoring the effect of the treatment comprises;
transforming the first eigenvector and each additional eigenvector into a separation distance; and
a reduction in separation distance between an earlier phase and a later phase of a treatment indicates an efficacy of the treatment.

16. The computer-implemented method of claim 13, wherein the plurality of microbial taxa and associated abundances includes at least one measurement selected from the group consisting of genomic measurements, gene expression measurements, proteomic measurements, and metabolite measurements.

17. A computer-implemented method for determining the effects of a therapeutic intervention associated with a gut microbiome of a group of subjects, the method comprising:
providing a microbiome dataset comprising a plurality of entries from before and after the therapeutic intervention, each entry comprising a plurality of microbial taxa and associated abundances, each entry further comprising at least one subject classification selected from an age, a health condition, a treatment condition, and a geographical location;
transforming a first portion of the microbiome dataset into a first eigenspectrum;
transforming at least one additional portion of the microbiome dataset into at least one additional eigenspectrum;
comparing corresponding components of the first eigenspectrum and the at least one additional eigenspectrum; and
determining the effects of a therapeutic intervention associated with a gut microbiome based on the comparison of the first eigenspectrum and the at least one additional eigenspectrum;
wherein each of the first eigenspectrum and the at least one additional eigenspectrum comprises a plurality of eigenvectors and associated eigenvalues.

18. The computer-implemented method of claim 17, wherein:
determining the effects of a therapeutic intervention associated with a gut microbiome comprises identifying a microbiome configuration age to achieve a stable microbiome configuration;
the first portion comprises a combination of all entries of the plurality of entries of the microbiome dataset comprising the subject classifications of the youngest age and the oldest age;
each additional portion comprises a successively larger portion of the plurality of entries, the successively larger portion comprising all entries of the plurality of entries of the microbiome dataset comprising the subject classifications of the youngest age, the oldest age, and successively larger portions of the ages between the youngest age and the oldest age;
comparing corresponding components of the first eigenspectrum and the at least one additional eigenspectrum comprises comparing each first eigenvalue associated with each first eigenvector of each eigenspectrum; and
determining the effects of a therapeutic intervention associated with a gut microbiome based on the comparison of the first eigenspectrum and the at least one additional eigenspectrum comprises:
identifying the stable eigenspectrum from the at least one additional eigenspectrum at which the first eigenvalue reaches an asymptotic value; and
identifying the age added to generate the additional portion of the entries transformed into the stable eigenspectrum as the age to achieve a stable microbiome configuration.

19. The computer-implemented method of claim 17, wherein:
determining the effects of the therapeutic intervention associated with a gut microbiome comprises a plurality of phases;
the first portion comprises a combination of all entries of the plurality of entries of the microbiome dataset with a health condition of healthy;
each additional portion comprises a combination of all entries of the plurality of entries with a health condition of gastrointestinal condition and a treatment condition classified as undergoing one phase of the plurality of phases of the treatment; and
determining the effects of the therapeutic intervention associated with a gut microbiome comprises;
transforming the first eigenvector and each additional eigenvector into a separation distance; and
a reduction in separation distance between an earlier phase and a later phase of a treatment indicates an effect of the therapeutic intervention.

20. The computer-implemented method of claim 17, wherein the abundances of microbial taxa are determined by analysis of fecal samples.
